(19) United States
(12) Patent Application Publication
(10) Pub. No.: US 2015/0126374 A1
(43) Pub. Date: May 7, 2015

(54) HYPERMETHYLATED GENE MARKERS FOR HEAD AND NECK CANCER

(71) Applicant: THE JOHNS HOPKINS UNIVERSITY, BALTIMORE, MD (US)

(72) Inventors: Joseph A. Califano, Baltimore, MD (US); Daria A. Gaykalova, Baltimore, MD (US)

(73) Assignee: THE JOHNS HOPKINS UNIVERSITY, BALTIMORE, MD (US)

(21) Appl. No.: 14/381,489

(22) PCT Filed: Feb. 28, 2013
(86) PCT No.: PCT/US2013/028421

§ 371 Date: Aug. 27, 2014

Related U.S. Application Data
(60) Provisional application No. 61/604,235, filed on Feb. 28, 2012.

(51) Int. Cl. C12Q 1/68

(52) U.S. Cl. CPC C12Q 1/6886; C12Q 2600/106; C12Q 2600/154; C12Q 2600/118
USPC 506/2; 435/6.11; 506/9; 435/287.2; 506/16

(57) ABSTRACT

Methods and kits for diagnosing or predicting head and neck squamous cell carcinoma (HNSCC) and for predicting responsivity to therapeutic regimens for treating HNSCC are disclosed.

### CROSS-REFERENCE TO RELATED APPLICATIONS

This application claims the benefit of U.S. Provisional Application No. 61/604,235, filed Feb. 28, 2012, which is incorporated herein by reference in its entirety.

### FEDERALLY SPONSORED RESEARCH OR DEVELOPMENT

This invention was made with government support under 1RC1DE020324-01 awarded by the National Institutes of Health (NIH). The government has certain rights in the invention.

### BACKGROUND

Head and Neck Squamous Cell Carcinoma (HNSCC) affects an estimated 50,000 individuals in the United States and 500,000 individuals worldwide annually (Jemal et al., 2011; Marur and Forastiere, 2008). HNSCC is a useful model for the study of human malignancy due to easy accessibility of primary tumor tissue, diverse etiology, and defined premalignant progression (Argiris et al., 2008; Ha and Califano, 2006; Marur and Forastiere, 2008). It also is useful as a model system for the study of epigenetic alterations in malignancy, due to the established role of epigenetic changes in pathogenesis (Ha and Califano, 2006; Mydlarz et al., 2010). Despite initial advances in the understanding of HNSCC biology, however, approximately half of all patients with HNSCC succumb to their disease (Ang et al., 2010; D'Souza et al., 2007; Jemal et al., 2011; Psyrri et al., 2011; Weinberger et al., 2006).

Current research is largely focused on genetic and epigenetic alterations resulting in the downregulation of tumor suppressor genes, such as p53, Rh, p16, DCC, TIMP3, EDNRB, and the upregulation of oncogenes, such as EGFR and CCND1 genes (Carvalho et al., 2006; Carvalho et al., 2011; Chung et al., 2004; Demokan et al., 2010; Ehrlich, 2002; Hardisson, 2003; Sun et al., 2012b). Several other genes, including cytoglobin, RASSF1A , SPARC, GSTM1, cyclinA1, MX1, WIF1, GNG7, and CYP1A1, have demonstrated high rates of DNA methylation in primary HNSCC tumors (Belbin et al., 2008; Calmon et al., 2009; Hartmann et al., 2012; He et al., 2010; He et al., 2011; Sharma et al., 2010; Shaw et al., 2006; Wang et al., 2011; Yang et al., 2011), and might be further validated for detection in bodily fluids.

The development of cancer-specific gene or regulatory regions and prospective target-specific therapies for HNSCC has so far been limited mostly to the genes cited above. Genome wide identification of epigenetically altered genes in HNSCC allows for the elucidation of mechanisms of carcinogenesis and the identification of novel potential therapeutic targets. In addition, genome-wide approaches can discover new cancer-specific DNA methylation events that can he used for molecular detection strategies in surgical margins or bodily fluids (Carvalho et al., 2011; Carvalho et al., 2008; Langbein et al., 2006; Sun et al., 2012a; Sun et al., 2012b). Previously published comprehensive whole-genome profiling approaches to promoter methylation in malignancies have been based on in vitro techniques that employ treatment of cultured cells with pharmacologic demethylating agents, such as 5-aza-2′-deoxycytidine (5-aza-dC), and subsequent expression array analysis with validation of tumor suppressor gene targets in primary tumors (Yamashita et al., 2002). This approach, however, often results in the lower rates of cancer-specific methylation, due to differences between the DNA methylation landscapes of cell lines and primary tissues (Hennessey et al., 2011), suggesting that clinically relevant data might only be available from analysis of primary tumor tissues.

### SUMMARY

In some aspects, the presently disclosed subject matter provides methods for diagnosing or predicting head and neck squamous cell carcinoma (HNSCC) in a subject baying or at risk of developing HNSCC, the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative that the subject has or is at risk of developing HNSCC.

In other aspects, the presently disclosed subject matter provides a method for determining the prognosis of a subject having head and neck squamous cell carcinoma (HNSCC), the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative of a poor prognosis in the subject having HNSCC.

In further aspects, the presently disclosed subject matter provides a method for predicting responsiveness to a therapeutic regimen for treating head and neck squamous cell carcinoma (HNSCC) in a subject in need of a therapeutic regimen thereof, the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative that the subject will be responsive to the therapeutic regimen for treating HNSCC.

In still further aspects, the presently disclosed subject matter provides a kit for diagnosing or predicting head and neck squamous cell carcinoma (HNSCC) in a subject having or at risk of developing HNSCC, the kit comprising: (a) a substrate for collecting a sample from the subject; and (b) means for determining the methylation state of a regulatory region of a gene in the sample, wherein the gene is selected from the group consisting of ZNF14, ZNF160, and ZNF420.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below

### BRIEF DESCRIPTION OF THE FIGURES

Having this described the presently disclosed subject matter in general terms, reference will now be made to the accompanying Figures, which are not necessarily drawn to scale, and wherein:

FIG. 1 shows an integrative strategy to identify genes that are candidate proto-oncogenes and tumor suppressors based on the hypothesis that such genes are transcriptionally activated and repressed in association with gene-specific promoter methylation alterations in a cohort of 44 primary HNSCC and 25 normal mucosal samples;

FIGS. 2a and 2b show COPA analysis of MAP4K1: (a) methylation COPA graph for MAP4K1, a candidate gene from the top 36 genes selected after integrative COPA analysis and correlation computation. Normals are demethylated as compared to tumors; and (b) expression COPA graph for MAP4K1. Expression is decreased in tumors as compared to normals;

FIG. 3 shows validation of 36 genes by bisulfite sequencing in 10 samples from the initial discovery cohort. Hemi methylation is depicted by grey and complete methylation is represented by black;

FIG. 4 shows validation of 20 candidates by bisulfite sequencing in a separate cohort of 32 HNSCC tumors and 16 normal samples;

FIGS. 5a-5d show validation of expression of zinc fingers in separate cohort by quantitative RT-PCR: ZNF149 (a), ZNF160 (h), ZNF420 (c), and ZNF585B (d) showed a significant difference in expression profiles while comparing normal with primary tissues. Expression was significantly higher in normal, which signifies association between tumor methylation and repression. All samples were normalized to GAPDH;

FIG. 6 shows a schematic outline of the presently disclosed integrative expression and methylation screening strategy, which combines high-throughput screening of DNA methylation and gene expression for the discovery cohort of HNSCC, two-tailed COPA, Spearman assays and several steps of candidate genes validation by bisulfite sequencing, qRT-PCR and QMPS on the original discovery and two additional validation cohorts;

FIG. 7 shows promoter DNA hypermethylation of the prospective tumor suppressing genes. Shown are the bisulfite sequencing results with associated p-values in 32 HNSCC rumor samples and 14 normal tissues from the first validation cohort for twenty top-scoring candidate genes. Shaded black boxes represent completely methylated promoters, gray boxes—semimethylated promoters, white boxes—completely unmethylated promoters. P-values were calculated by Fisher's exact test comparing the number of methylated and semimethylated promoters vs. unmethylated promoters in tumor vs. normal samples. None (*) for MAP4K1 p-values was calculated comparing the number of methylated promoters vs., unmethylated and semimethylated promoters in tumor vs. normal, due to the high level of the methylation. ND=p-value was not calculated because the methylation status of the genes in tumors was unchanged or rather hypomethylated contradiction the original discovery and validation data (see also FIG. 11);

FIG. 8 shows ZNF gene expression downregulation by DNA methylation. Shown are the qRT-PCR results with associated p-values in 32 HNSCC tumor samples and 14 normal tissues from the first validation cohort for five ZNF protein genes. ZNF expression was quantified relative to GAPDH expression. P-values were calculated by t-test comparing the tumor vs. normal samples. While four ZNF showed significant downregulation of gene expression on the subset of tumor samples, ZNF71 demonstrated relative upregulation of gene expression in tumor samples;

FIG. 9 shows ZNF14, ZNF160, ZNF420 DNA methylation detection in bodily fluids of HNSCC patients. Shown are ZNF QMSP results in 59 HNSCC primary tumor and salivary rinse compared to normal plasma and salivary rinse samples from the second validation cohort. ZNF promoter methylation was quantified relative to BACT methylation and multiplied by 100. NS stands for normal salivary rinse sample (n=35), N—normal primary tissues (n=31), TS—salivary rinse from the HNSCC patients (n=59), T—primary tumor samples from HNSCC patients (n=59). Note that no detectable level of ZNF DNA methylation is observed in normal samples;

FIG. 10 shows the separate COPA plots for the methylation and expression of the 36 candidate genes from the discovery cohort. For each indicated gene methylation panel is on the left and expression panel is on the right. Blue indicates normal samples and red tumor samples. For each panel the same samples are plotted, but order will differ based on individual data type as they are ordered from smallest to largest value;

FIG. 11 shows promoter DNA hypermethylation of 36 candidate prospective tumor suppressing genes from the discovery cohort. Shown are the bisulfite sequencing results in 5 HNSCC tumor samples and 5 normal tissues picked from the original discovery cohort for 36 top-scoring candidate genes. Shaded black boxes represent completely methylated promoters, gray boxes —semimethylated promoters, white boxes—completely unmethylated promoters. P-values were not calculated due to the small number of samples;

FIGS. 12A-12C show that the expression of ZNF14, ZNF160 and ZNF420 is coordinately associated with methylation status of individual promoter CpG islands in Human Head and Neck Cell lines. Top panel of A (ZNF14), B (ZNF160) and C (ZNF420) shows relative to GAPDH expression of the gene in the individual cell line. The bottom panel is a matched bisulfate sequencing results. Shaded black boxes represent completely methylated promoters, gray boxes—semimethylated promoters, white boxes—completely unmethylated promoters;

FIGS. 13A-13I show functional analysis of ZNF14, ZNF160 and ZNF420 in Head and Neck cell lines. ZNF14 (A-C), ZNF160 (D-F) and ZNF420 (G-I) were either temporary ectopically expressed in cancer cell lines (A-B, D-E, G-H) or knock-down in the normal keratynocyte cells (c, F, I). Cell proliferation rate was measured relative to empty vector (EV) or scrambled shRNA sequence by CCK-8 kit every 24 hours after transfection. Experiment was repeated in pentaplicates; and

FIG. 14 shows the Kaplan-Meier Curve of overall survival by ZNF methylation status or other clinical risk factors. Overall survival was defined from the end of after surgery therapy to the date of last follow up or date of death. P-value indicated the results from cox proportional hazard model.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Hypermethylated Gene Markers for Head and Neck Cancer

Genome wide identification of epigenetically altered genes in HNSCC will elucidate mechanisms of carcinogenesis and identify novel potential therapeutic targets. In addition, this approach may be useful for molecular detection strategies because tumor specific expressed genes can be uniquely expressed in malignancy and may be detected in surgical margins or body fluids. A direct comprehensive genome wide integrated analysis of epigenetic and transcriptional alteration in primary HNSCC has not been performed before. Such data can be beneficial for the development of individual targeted therapy.

Previously published comprehensive whole-genome profiling approaches to promoter methylation in malignancies have been based on biased in vitro techniques that employ treatment of cultured cells with pharmacologic demethylating agents and subsequent expression array analysis with validation of tumor suppressor gene targets in primary tumors. To date, only a few examples of promoter hypomethylation causing unmasked expression of candidate proto-oncogenes have been reported. To avoid this bias, a high throughput approach was devised using expanded expression and methylation arrays. Without wishing to be bound to any one particular theory, it was thought that proto-oncogenes and tumor suppressor genes are transcriptionally activated and repressed, respectively, in association with gene-specific promoter methylation alterations. Previous studies using pharmacologic demethylation in normal, minimally-transformed oral-keratinocyte cell lines combined with COPA analysis of expression in primary tissues as a discovery approach, defined a set of candidate proto-oncogenes that undergo aberrant demethylation and increased expression in primary human tumors.

These data indicate that aberrant demethylation of multiple, physiology repressed proto-oncogenes and cancer testis antigens occur in human cancers in a coordinated fashion in individual tumors. Hence a novel integrative strategy was devised to identify putative oncogenes and tumor suppressors by manipulating the traditional Cancer Profile outlier analysis (COPA) to a two-sided COPA analysis that can, in addition to discovery of oncogenes, also identify prospective tumor suppressors.

Accordingly, the presently disclosed subject matter determined significantly altered genes that are associated with primary HNSCC in an expanded expression array containing 1.4 million probes and directly defined aberrant methylation marks in primary HNSCC using a 27K promoter methylation specific DNA array, and identified significant individual gene specific methylation-transcription correlations.

General embodiments of the presently disclosed subject matter relate to methods and kits used for diagnosing, or evaluating a subject having or at risk of developing head and neck cancer by determining the methylation state of a gene or the regulatory region of at least one gene in a nucleic acid sample from the subject, and wherein at least one gene or regulatory region is hypermethylated as compared to the same region in a corresponding normal cell.

More particularly, in some embodiments, the presently disclosed subject matter provides a method for diagnosing or predicting head and neck squamous cell carcinoma (HNSCC) in a subject having or at risk of developing HNSCC. In other embodiments, the presently disclosed subject matter is directed to methods and compositions for determining the prognosis of a patient having a cellular proliferative disorder. In further embodiments, the presently disclosed subject matter is directed to methods and compositions for predicting responsiveness to a therapeutic regimen for treating a cellular proliferative disorder in a subject in need of a therapeutic regimen thereof. In yet further embodiments, the presently disclosed subject matter is directed to methods and compositions for diagnosing or predicting a cellular proliferative disorder in a subject. In other embodiments, the presently disclosed subject matter provides a kit for diagnosing or predicting head and neck squamous cell carcinoma (HNSCC) in a subject having or at risk of developing HNSCC.

Examples of references describing methods for detecting a cellular proliferative disorder, such as HNSCC, by determining the methylation state of at least one gene or regulatory region of a gene include U.S. Pat. Nos.: 7,214,485; 7,153,657; 7,153,653; 6,893,820; 6,811,982; and 6,617,434.

### A. Methods for Diagnosing or Predicting a Cellular Proliferative Disorder

Generally, in some embodiments, the presently disclosed subject matter provides a method for diagnosing a disorder in a subject having or at risk of developing a cell proliferative disorder. The method includes contacting a nucleic acid-containing sample from cells of the subject with an agent that provides a determination of the methylation state of at least one regulatory region of a gene, wherein the at least one regulatory region is hypermethylated in a cell undergoing unregulated cell growth as compared to a corresponding normal cell; and identifying hypermethylation of the regulatory region in the nucleic acid-containing sample, as compared to the same region of the at least one regulatory region in a subject not having the proliferative disorder, wherein hypermethylation is indicative of a subject having or at risk of developing the proliferative disorder.

In some embodiments, the presently disclosed subject matter provides a method for diagnosing or predicting a cellular proliferative disorder in a subject, the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative that the subject has or is at risk of developing a cellular proliferation disorder.

Representative, non-limiting examples of cellular proliferative disorders include, but are not limited to, non-small cell lung cancer, head and neck carcinoma, lymphoma, melanoma, myeloma, neuroblastoma, glioblastoma, ovarian cancer, pancreatic cancer, prostate cancer, urothelial cancer, breast cancer, colon cancer, thyroid cancer, testicular cancer, tumors of the oral cavity, larynx, pharynx, neck, skull base, salivary glands, and premalignant conditions of the upper aerodigestive tract.

In particular embodiments, as provided immediately herein below, the cellular proliferative disorder includes head and neck squamous cell carcinoma (HNSCC).

### B. Methods for Diagnosing or Predicting Head and Neck Squamous Cell Carcinoma (HNSCC)

In some embodiments, the presently disclosed subject matter provides a method for diagnosing or predicting head and neck squamous cell carcinoma (HNSCC) in a subject having or at risk of developing HNSCC, the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative that the subject has or is at risk of developing HNSCC.

In an embodiment, specific, gene sequences that can he used in the methods and kits of the presently disclosed subject matter include NCBI GeneID 7561 (ZNF14), GeneID 90338 (ZNF160) and GeneID 147923 (ZNF420). In a further embodiment, other related sequences that encode for ZNF14, ZNF160, and ZNF420 also can be used in the presently disclosed methods.

One embodiment of the presently disclosed subject matter is based on the testing and identification of a unique profile of gene promoters that are effective markers for risk of HNSCC. The profiles comprise any of the specified genes alone, or in combination with each other or other non-listed or unknown yet to be discovered gene promoters. The gene promoter panels comprise from 2 to 25 genes or regulatory regions of genes. Preferably the panel will include at least one differentially methylated gene or regulatory region of a gene selected from the group consisting of ZNF1.4, ZNF160, and ZNF420, This panel creates an improved ability to detect epigenetic changes associated with HNSCC in salivary rinses and serum from patients with HNSCC.

As provided herein, hypermethylation may occur in the gene or regulatory region thereof in some embodiments, the hypermethylation occurs within the regulatory region of the genes identified herein, and, in particular embodiments, the hypermethylation is in the promoter sequence of the regulatory region. In some embodiments, the regulatory region is a promoter. More particularly, the hypermethylation may be in a CpG dinucleotide motif of the promoter.

In some embodiments, the hypermethylation of the regulatory region is determined by detecting decreased expression of the gene. In certain embodiments, the decreased expression of the gene is detected by reverse transcription-polymerase chain reaction (RT-PCR). In further embodiments, the hypermethylation of the regulatory region is determined by detecting decreased mRNA of the gene. In yet further embodiments, the hypermethylation of the regulatory region is determined by detecting decreased protein encoded by the gene. Typically, expression is assessed and compared in test samples and control samples which may be normal, non-malignant cells. The test samples may contain cancer cells or pre-cancer cells or nucleic acids from them.

In more particular embodiments, the hypermethylation of the regulatory region determined by contacting at least a portion of the regulatory region with a methylation-sensitive restriction endonuclease, the endonuclease preferentially cleaving non-methylated recognition sites relative to methylated recognition sites, whereby cleavage of the portion of the regulatory region indicates non-methylation of the portion of the regulatory region provided that the regulatory region comprises a recognition site for the methylation-sensitive restriction endonuclease. In an embodiment, methylation-sensitive restriction endonucleases can be used to detect methylated CpG dinucleotide motifs. Examples of endonucleases that preferentially cleave methylated recognition sites relative to non-methylated recognition sites include, but are not limited to, Ace III, Ban I, BstN I, Msp I, and Xma I. Examples of endonucleases that preferentially cleave non-methylated relative to methylated recognition sites include, but are not limited to, Ace II, Ava I, BssH II, BstU I, Hpa II, and Not I.

Alternatively, chemical reagents can be used which selectively modify either the methylated or non-methylated form of CpG dinucleotide motifs. In yet more particular embodiments, the hypermethylation of the regulatory region is determined by: (a) contacting at least a portion of the regulatory region with a chemical reagent that selectively modifies a non-methylated cytosine residue relative to a methylated cytosine residue, or selectively modifies a methylated cytosine residue relative to a non-methylated cytosine residue; and (b) detecting a product generated by the contacting step. In further embodiments, the step of detecting comprises hybridization with at least one probe that hybridizes to a sequence comprising a modified non-methylated CpG dinucleotide motif but not to a sequence comprising an unmodified methylated CpG dinucleotide. In yet further embodiments, the step of detecting comprises amplification with at least one primer that hybridizes to a sequence comprising a modified non-methylated CpG dinucleotide motif but not to a sequence comprising an unmodified methylated CpG dinucleotide motif thereby forming amplification products. In yet further embodiments, the step of detecting comprises amplification with at least one primer that hybridizes to a sequence comprising an unmodified methylated CpG dinucleotide motif but not to a sequence comprising a modified nonmethylated CpG dinucleotide motif thereby forming amplification products.

Specific primers and probes for the presently disclosed subject matter are disclosed herein. As such, the methods may comprise these specific primers and probes. In an embodiment, the steps of the presently disclosed subject matter further comprise using at least one of primers or probes disclosed herein. As illustrated in the Examples herein, in some embodiments, analysis of methylation can be performed by bisulfite genomic sequencing. Bisulfite ions, for example, sodium bisulfite, convert non-methylated cytosine residues to bisulfite modified cytosine residues. The bisulfite ion treated gene sequence can be exposed to alkaline conditions, which convert bisulfite modified cytosine residues to uracil residues. Sodium bisulfite reacts readily with the 5,6-double bond of cytosine but poorly with methylated cytosine) to form a sulfonated cytosine reaction intermediate that is susceptible to deamination, giving rise to a sulfonated uracil. The sulfonate group can be removed by exposure to alkaline conditions, resulting in the formation of uracil. The DNA can be amplified, for example, by PCR, and sequenced to determine whether CpG sites are methylated in the DNA of the sample. Uracil is recognized as a thymine by Taq polymerase and, upon PCR, the resultant product contains cytosine only at the position where 5-methylcytosine was present in the starting template DNA.

One can compare the amount or distribution of uracil residues in the bisulfite ion treated gene sequence of the test cell with a similarly treated corresponding non methylated gene sequence. A decrease in the amount or distribution of uracil residues in the gene from the test cell indicates methylation of cytosine residues in CpG dinucleotides in the gene of the test cell. The amount or distribution of uracil residues also can be detected by contacting the bisulfite ion treated target gene sequence, following exposure to alkaline conditions, with an oligonucleotide that selectively hybridizes to a nucleotide sequence of the target gene that either contains uracil residues or that lacks uracil residues, but not both, and detecting selective hybridization (or the absence thereof) of the oligonucleotide.

In another embodiment, the gene is contacted with hydrazine, which modifies methylated cytosine residues. The hydrazine treated gene sequence then is contacted with a reagent, such as piperidine, which cleaves the nucleic acid molecule at hydrazine modified cytosine residues, thereby generating a product comprising fragments. By separating the fragments according to molecular weight, using, for example, an electrophoretic, chromatographic, or mass spectrographic method, and comparing the separation pattern with that of a similarly treated corresponding non-methylated gene sequence, gaps are apparent at positions in the test gene contained methylated cytosine residues. As such, the presence of gaps is indicative of methylation of a cytosine residue in the CpG dinucleotide in the target gene of the test cell.

Modified products can be detected directly, or, after a further reaction, which creates products that are easily distinguishable. Means for detecting altered size and/or charge can be used to detect modified products, including, but not limited to electrophoresis, hybridization, amplification, primer extension, sequencing, ligase chain reaction, chromatography, mass spectrometry, and combinations thereof.

In other embodiments, hypermethylation can be identified through nucleic acid sequencing after bisulfite treatment to determine whether a uracil or a cytosine is present at specific location within a gene or regulatory region. If uracil is present after bisulfite treatment, then the nucleotide was unmethylated. Hypermethylation is present when there is a measurable increase in methylation.

In an alternative embodiment, the method for analyzing methylation of the target gene can include amplification using a primer pair specific for methylated residues within the target gene. In these embodiments, selective hybridization or binding of at least one of the primers is dependent on the methylation state of the target DNA sequence. For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

In an embodiment, methylation status can be assessed using real-time methylation specific PCR. For example, the methylation level of the promoter region of one or more of the target genes can be determined by determining the amplification level of the promoter region of the target gene based on amplification-mediated displacement of one or more probes whose binding sites are located within the amplicon. In general, real-time quantitative methylation specific PCR is based on the continuous monitoring of a progressive fluorogenic PCR by an optical system. Such PCR systems are well-known in the art and usually use two amplification primers and an additional amplicon-specific, fluorogenic hybridization probe that specifically binds to a site within the amplicon.

The probe can include one or more fluorescence label moieties. For example, the probe can be labeled with two fluorescent dyes: (1) a 6-carboxy-fluorescein (FAM), located at the 5 ′-end, which serves as reporter; and (2) a 6-carboxy-tetramethyl-rhodamine (TAMRA), located at the 3′-end. Which serves as a quencher. When amplification occurs, the 5′-3′ exonuclease activity of the Tag DNA polymerase cleaves the reporter from the probe during the extension phase, thus releasing it from the quencher. The resulting increase in fluorescence emission of the reporter dye is monitored during the PCR process and represents the number of DNA fragments generated.

In particular embodiments, hypermethylation of the regulatory region is determined using quantitative methylation-specific PCR (QMSP). Methods using an amplification reaction can utilize a real-time detection amplification procedure. For example, the method can utilize molecular beacon technology.

In further embodiments, methyl light (Trinh B N, Long T I, Laird P W. 25(4):456-62 (2001), incorporated herein in its entirety by reference), Methyl Heavy (Epigenomics, Berlin, Germany), or SNuPE (single nucleotide primer extension) (See e.g., Watson D., et al., Genet Res. 75(3):269-74 (2000)) can be used in the presently disclosed methods related to identifying altered methylation of the genes or regulatory regions provided herein. Additionally, methyl light, methyl heavy, and array-based methylation analysis can be performed by using bisulfite treated DNA that is then PCR-amplified, against microarrays of oligonucleotide target sequences with the various forms corresponding to unmethylated and methylated DNA.

The degree of methylation in the DNA associated with the gene or genes or regulatory regions thereof, may he measured by fluorescent in situ hybridization (FISH) by means of probes which identify and differentiate between genomic DNAs, which exhibit different degrees of DNA methylation. FISH is described in the Human chromosomes: principles and techniques (Editors, Ram S. Verma, Arvind Babu Verma, Ram S.) 2nd ed., New York: McGraw-Hill, 1995, which is incorporated herein by reference in its entirety. In such embodiments, the biological sample will typically be any which contains sufficient whole cells or nuclei to perform short term culture. Usually, the sample will be a tissue sample that contains 10 to 10,000, or, for example, 100 to 10,000, whole somatic cells.

Other methods are known in the art for determining methylation status of a target gene, including, but not limited to, array-based methylation analysis and Southern blot analysis.

Methods employing hybridization to nucleic acid probes can be employed for measuring specific mRNAs. Such methods include using nucleic acid probe arrays (microarray technology), in situ hybridization, and using Northern blots. Messenger RNA also can be assessed using amplification techniques, such as RL-PCR.

As known in the art, in nucleic acid hybridization reactions, the conditions used to achieve a particular level of stringency will vary, depending on the nature of the nucleic acids being hybridized. For example, the length, degree of complementarity, nucleotide sequence composition (for example, relative GC: AT content), and nucleic acid type, i.e., whether the oligonucleotide or the target nucleic acid sequence is DNA or RNA, can be considered in selecting hybridization conditions. An additional consideration is whether one of the nucleic acids is immobilized, for example, on a filter. Methods for selecting appropriate stringency conditions can be determined empirically or estimated using various formulas, and are well known in the art (see, for example, Sambrook et al., supra, 1989).

An example of progressively higher stringency conditions is as follows: 2×SSC0.1% SDS at about room temperature (hybridization conditions); 0.2×SSC0.1% SDS at about room temperature (low stringency conditions); 0.2×SSC/0.1%SDS at about 420 C (moderate stringency conditions); and 0.1.×SSC at about 68° C. (high stringency conditions). Washing can be carried out using only one of these conditions, for example, high stringency conditions, or each of the conditions can be used, for example, for 10 to 15 minutes each, in the order listed above, repeating any or all of the steps listed.

Advances in genomic technologies now permit the simultaneous analysis of thousands of genes, although many are based on the same concept of specific probe target hybridization. Sequencing-based methods are an alternative; these methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest. Moreover, specific proteins can be assessed using any convenient method including immunoassays and immuno-cytochemistry but are not limited to that. Most such methods will employ antibodies that are specific for the particular protein or protein fragments. The sequences of the mRNA (cDNA) and proteins of the target genes of the presently disclosed subject matter are known in the art and publicly available.

Samples for use in the presently disclosed methods and compositions can include any biological sample from the subject. The biological sample can be a tissue sample which contains from, in some embodiments, 1 to 10,000,000, in other embodiments, 1000 to 10,000,000, or, in yet other embodiments, 1,000,000 to 10,000,000 somatic cells. It is possible, however, to obtain samples that contain smaller numbers of cells, even a single cell in embodiments that utilize an amplification protocol, such as PCR. The sample need not contain any intact cells, so long as it contains sufficient material (e.g., protein or genetic material, such as RNA or DNA) to assess methylation status or gene expression levels.

In some embodiments the sample is selected from the group consisting of a tissue sample, a frozen tissue sample, a biopsy specimen, a surgical specimen, including a specimen from a surgical margin, a cytological specimen, whole blood, bone marrow, cerebral spinal fluid, peritoneal fluid, pleural fluid, lymph fluid, serum, mucus, plasma, urine, chyle, stool, ejaculate, sputum, nipple aspirate, and saliva, in particular embodiments, the sample is a saliva sample. In other embodiments, the methods and kits of the presently disclosed subject matter use both serum and saliva, as well as either of them alone.

A sample for use with the presently disclosed methods and compositions may be a biological or tissue sample drawn from any tissue that is susceptible to cancer. For example, the tissue may be obtained by surgery, biopsy, swab, stool, or other collection method. The biological sample may be, for example, a sample from colorectal tissue or, in certain embodiments, can be a blood sample, or a fraction of a blood sample such as a peripheral blood lymphocyte (PBL) fraction. Methods for isolating RBLs from whole blood are well known in the art. In addition, it is possible to use a blood sample and enrich the small amount of circulating cells from a tissue of interest, e.g., lung, colon, breast, and the like, using a any method known in the art.

### C. Methods or Determining the Prognosis of a Subject Having Head and Neck Squamous Cell Carcinoma

In an embodiment of the presently disclosed subject matter, there are provided methods of determining the prognosis of a subject having a cell proliferative disorder. The method includes determining the methylation state of at least one regulatory region of a gene in a nucleic acid sample from the subject, wherein hypermethylation as compared to a corresponding normal cell in the subject or a subject not having the disorder, is indicative of a poor prognosis.

More particularly, the presently disclosed subject matter provides methods for determining the prognosis of a subject having HNSCC. In an embodiment, the method is a method for determining the prognosis of a subject having, head and neck squamous cell carcinoma (HENSCC), the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative of a poor prognosis in the subject having HNSCC.

In another embodiment, the method comprises determining the methylation states of regulatory regions of two or more genes in the sample and comparing the methylation states of the regulatory regions of the two or more genes in the sample to the methylation states of the regulatory regions of the two or more genes in a control sample, wherein the two or more genes are selected from the group consisting of ZNF14, ZNF160, ZNF420, and a combination thereof.

In an embodiment, the sample is a saliva sample. In another embodiment, the regulatory region is a promoter. In a further embodiment, hypermethylation of the regulatory region is at a CpG dinucleotide motif.

Another embodiment discloses a panel of promoter hypermethylation markers that have created an improved ability to detect epigenetic changes associated with HNSCC in salivary rinses and serum from patients with HNSCC, wherein at least one marker in the panel is selected from the group consisting of ZNF14, ZNF160, and ZNF420. Further, this panel of promoter hypermethylation markers can be used to anticipate the diagnosis of tumor recurrence by detecting the epigenetic changes associated with HNSCC.

### D. Methods for Predicting Responsiveness to a Therapeutic Regimen for Treating Head and Neck Squamous Cell Carcinoma

The presently disclosed subject matter provides a method for predicting responsiveness to a therapeutic regimen for treating head and neck squamous cell carcinoma (HNSCC) in a subject in need thereof, the method comprising: (a) obtaining a sample from the subject; (b) determining the methylation state of a regulatory region of a gene in the sample, wherein the gene is selected from the group consisting of ZNF14, ZNF160, and ZNF420; and (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative that the subject will be responsive to the therapeutic regimen for treating HNSCC.

In an embodiment, the method comprises determining the methylation states of regulatory regions of two or more genes in the sample and comparing the methylation states of the regulatory regions of the two or more genes in the sample to the methylation states of the regulatory regions of the two or more genes in a control sample, wherein the two or more genes are selected from the group consisting of ZNF14. ZNF160, ZNF420, and a combination thereof.

In another embodiment, the sample is a saliva sample. In another embodiment, the regulatory region is a promoter. In a further embodiment, hypermethylation of the regulatory region is at a CpG dinucleotide motif.

In a certain embodiment, the therapeutic regimen for treating HNSCC comprises administration of a chemotherapeutic agent. In still another embodiment, the chemotherapeutic agent is selected from the group consisting of methotrexate, cisplatin/carboplatin, canbusil, dactinomicin, taxol (paclitaxol), a vinca alkaloid, a mitomycin-type antibiotic, a bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxy daunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, and mechlorethamine.

In an embodiment, the therapeutic regimen for treating HNSCC comprises administration of a demethylating agent. In another embodiment, the demethylating agent is selected from the group consisting of 5-azacytidine, 5-aza-2-deoxycytidine, and zebularine. In a further embodiment, the therapeutic regimen for treating HNSCC comprises administration of a chemotherapeutic agent in combination with a demethylating agent.

### E. Kits for Diagnosing or Predicting Head and Neck Squamous Cell Carcinoma

In another embodiment, the presently disclosed subject matter provides a kit for detecting a cellular proliferative disorder in a subject comprising one or more reagents for detecting the methylation state of at least one gene or regulatory region associated with ZNF14, ZNF160, and/or ZNF420.

In a particular embodiment, the presently disclosed subject matter includes a kit for diagnosing or predicting head and neck squamous cell carcinoma (HNSCC) in a subject having or at risk of developing HNSCC, the kit comprising: (a) a substrate for collecting a sample from the subject; and (b) means for determining the methylation state of a regulatory region of a gene in the sample, wherein the gene is selected from the group consisting of ZNF14, ZNF160, and ZNF420.

In an embodiment, the kit comprises a means for determining the methylation states of regulatory regions of two or more genes in the sample, wherein the two or more genes are selected from the group consisting of ZNF14, ZNF160, ZNF420, and a combination thereof.

In another embodiment, the kit includes an agent that provides a determination of the methylation state of a gene or the regulatory region of at least one gene, and a panel of one or more genes selected from ZNF14, ZNF160, and ZNF420.

An additional embodiment features a kit, for practicing any of the methods described herein, including an agent that provides a determination of the methylation state of a gene or the regulatory region of at least one gene; and a panel of two or more genes selected from ZNF14, ZNF160, and ZNF420.

In another embodiment, the sample is a saliva sample. In a further embodiment, the regulatory region is a promoter. In still another embodiment, hypermethylation of the regulatory region is at a CpG dinucleotide motif.

In a further embodiment, the kit is any article of manufacture (e.g., a package or a container) comprising a substrate for collecting a biological sample from the patient and means for measuring the levels of one or more hypermethylated genes or regulatory regions of a gene as described herein.

In certain embodiments, a patient can be diagnosed by adding a sample from the patient to the kit and detecting the relevant gene or regulatory regions. The method may comprise the steps of collecting the sample from a patient, adding the sample from the patient to a diagnostic kit, and detecting the hypermethylated genes or regulatory regions of a gene. The sample may include blood, blood serum, saliva, or any other part of the patient that can be assayed for hypermethylated genes or regulatory regions of a gene. In other kit and diagnostic embodiments, the sample need not be collected from the patient because it is already collected.

In other embodiments, the kit can also comprise a washing solution or instructions for making a washing solution, in which the combination of the capture reagents and the washing solution allows capture of a hypermethylated gene or regulatory regions of a gene on the solid support for subsequent detection. In further embodiments, a kit can comprise instructions in the form of a label or separate insert. For example, the instructions may give information regarding how to collect the sample, or the particular hypermethylated gene or regulatory regions of a gene to be detected, and the like. In yet another embodiment, the kit can comprise one or more containers with hypermethylated gene or regulatory region of a gene samples that can be used as standard(s) for calibration.

### F. Methods for Treating a Patient for Head and Neck Squamous Cell Carcinoma

The presently disclosed subject matter also provides methods for treating a patient with or at risk for HNSCC. In an embodiment, the presently disclosed subject matter provides a method for treating head and neck squamous cell carcinoma (HNSCC) in a subject in need thereof, the method comprising: (a) obtaining a sample from the subject; (b) determining the methylation state of a regulatory region of at least one gene in the sample, wherein the gene is selected from the group consisting of ZNF14, ZNF160, and ZNF420; (c) comparing the methylation state of the regulatory region of the gene in the sample to the methylation state of the regulatory region of the gene in a control sample; wherein hypermethylation of the regulatory region of the gene in the sample as compared to the regulatory region of the gene in the control sample is indicative that the subject should be treated for HNSCC; and (d) administering a drug to the subject to prevent or treat HNSCC.

In a certain embodiment, treating the subject for HNSCC comprises administration of a chemotherapeutic agent. In still another embodiment, the chemotherapeutic agent is selected from the group consisting of methotrexate, cisplatin/carboplatin, canbusil, dactinomicin, taxol (paclitaxol), a vinca alkaloid, a mitomycin-type antibiotic, a bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxy daunorubicin, 13-deoxydaunorubicin adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine., vindesine, tamoxifen, and mechlorethamine.

In an embodiment, treating the subject for HNSCC comprises administration of a demethylating agent. In another embodiment, the demethylating agent is selected from the group consisting of 5-azacytidine, 5-aza-2-deoxycytidine, and zebularine.

In a further embodiment, treating the subject for HNSCC comprises administration of a chemotherapeutic agent in combination with a demethylating agent.

In still another embodiment, before treating the subject for HNSCC, the physician treating the subject performs additional testing to confirm the diagnosis of HNSCC.

### II. Definitions

As used herein, the term “comparing” refers to making an assessment of how the proportion, level or cellular localization of one or more genes or regulatory regions of a gene in a sample from a patient relates to the proportion, level or cellular localization of one or more genes or regulatory regions of a gene in a control sample. For example, “comparing” may refer to assessing whether the proportion, level, or cellular localization of one or more hypermethylated genes or regulatory regions of a gene in a sample from a patient is the same as, more or less than, or different in proportion, level, or cellular localization of the corresponding one or more hypermethylated genes or regulatory regions of a gene in a standard or control sample.

DNA methylation is a biochemical process involving the addition of a methyl group to the cytosine or adenine nucleotides. As used herein, a “hypermethylated gene or regulatory region of a gene” is any gene or regulatory region of a gene that is more methylated compared to that of a gene or regulatory region of a gene found in a normal or healthy cell or tissue.

As used herein, “semi methylated” or “hemi methylated” means that only one of the two DNA strands in the duplex DNA is methylated. Alternatively, it can mean that only some of the nucleotides that can be methylated are actually methylated.

As used herein, the term “regulatory region” of a gene refers to a DNA sequence either upstream (i.e., at its 5′ end) or downstream (i.e., at its 3′ end) of the gene and operably linked to the gene such that it is able to exert an effect on transcription of the gene. In particular embodiments, the regulatory region is a promoter.

The term “cellular proliferative disorder” as used herein refers to malignant as well as non-malignant cell populations which often differ from the surrounding tissue both morphologically and genotypically. In some embodiments, the cellular proliferative disorder is a cancer. In particular embodiments the cancer may be a carcinoma or a sarcoma. A cancer can include, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas. In one aspect, the cancer is head and neck cancer. In particular embodiments, the head and neck cancer is head and neck squamous cell carcinoma (HNSCC).

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. It will be appreciated that, although not precluded, treating a disease, disorder or condition does not require that the disease, disorder, condition or symptoms associated therewith be completely eliminated.

As used herein, the terms “measuring” and “determining” refer to methods which include detecting the level of a gene or regulatory region(s) in a sample and/or the level of hypermethylation of a gene or a regulatory region(s).

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition.

As used herein, the term “subject at risk” of getting a disease refers to estimating that a subject will have a disease or disorder in the future based on the subject's current symptoms, family history, lifestyle choices, and the like.

As used herein, the term “indicative” or “likely” means that the event referred to is probable.

As used herein, the term “diagnosing” refers to the process of attempting to determine or identify a disease or disorder.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for treating an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

As used herein, the term “control sample”, “corresponding control”, or “appropriate control” means any control or standard familiar to one of ordinary skill the art useful for comparison purposes.

As used herein, the term “level of expression” of a gene or regulatory region refers to the amount of gene or regulatory region detected. Levels of gene or regulatory region can be detected at the transcriptional level, the translational level, and the post-translational level, for example.

As used herein, the term “selective hybridization” or “selectively hybridize” refers to hybridization under moderately stringent or highly stringent physiological conditions, which can distinguish related nucleotide sequences from unrelated nucleotide sequences. The term “nucleic acid molecule” is used broadly herein to mean a sequence of deoxyribonucleotides or ribonucleotides that are linked together by a phosphodiester bond. “Nucleic acid molecule” is meant to include DNA and RNA, which can be single stranded or double stranded, as well as DNA/RNA hybrids. Furthermore, the term “nucleic acid molecule” as used herein includes naturally occurring nucleic acid molecules, which can be isolated from a cell, for example, a particular gene of interest, as well as synthetic molecules, which can be prepared, for example, by methods of chemical synthesis or by enzymatic methods such as by the polymerase chain reaction (PCR), and, in various embodiments, can contain nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also can encode a peptide. As such, the different terms are used primarily for convenience of discussion.

The terms “target gene” or “target sequence” are used herein to refer to the gene or sequence that are or are thought to he differentially methylated. In certain embodiments, the “target sequence” is the specific sequence that a primer binds to for amplification.

A polynucleotide or oligonucleotide comprising naturally occurring nucleotides and phosphodiester bonds can be chemically synthesized or can he produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

As used herein, the terms “significantly different” or “significant difference” mean a level of expression of a gene or regulatory region of a gene or level of hypermethylation of a gene or regulatory region of a gene in a sample that is higher or lower than the level of expression of said gene or regulatory region of a gene or hypermethylation of said gene or regulatory region of a gene in a control sample by at least 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold or more.

As used herein, the term “effective” means amelioration of one or more causes or symptoms of a disease or disorder. Such amelioration only requires a reduction or alteration, not necessarily elimination, of said causes or symptoms.

As used herein, the term “antibody” is used in the broadest sense and encompasses naturally occurring forms of antibodies and recombinant antibodies such as single-chain antibodies, chimeric and humanized antibodies and multi-specific antibodies as well as fragments and derivatives of all of the foregoing.

The presently disclosed methods can be used to evaluate existing and new therapies t vitro, in vivo, or ex vivo. In some embodiments, the methods can be used to screen drugs in cell culture. For example, a cell can be contacted with a potential therapeutic drug and at least one hypermethylated gene or regulatory region of a gene disclosed herein can be assayed for the amount of hypermethylation. In some embodiments, the methods can be used to screen for new protocols or drugs in a subject by monitoring the gene or regulatory regions disclosed herein.

Unless defined otherwise, all technical and scientific terms used herein have the meaning commonly understood by a person skilled in the art to which this invention belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to he non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments 20%, in some embodiments ,±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods and Materials

### Histopathology

All samples were analyzed by the pathology department at Johns Hopkins Hospital. Tissues were obtained via Johns Hopkins Institutional Review Board approved protocols under JHM IRP Protocol #92-07-21-01, “Detection of Genetic Alterations IN Head & Neck Tumors.” Tumors were obtained from surgical resection and Normal mucosal tissues from Uvulopalato-pharyngoplasty procedures and immediately frozen in liquid nitrogen and stored at −80° C. until use. Tumor samples were confirmed to be HNSCC and subsequently microdissected to separate tumor from stromal cells to yield at least 75% tumor cells. Cohort characteristics are listed in Table 1.

TABLE 1
Cohort characteristics for 44 primary
HNSCC and 25 normal mucosal samples

| HNSCC (n = 44) | |
|---|---|
| Age | 58 ± 13 |
| Sex, % | |
| Male | 73 |
| Female | 27 |
| Race, % | |
| Caucasian | 91 |
| African american | 1 |
| Others | 2 |
| Smoking status, % | |
| Smokers | 61 |
| non-smokers | 28 |
| unknown | 11 |
| Drinking status, % | |
| Drink | 57 |
| Do not drink | 27 |
| Unknown | 16 |
| Site, % | |
| Oral cavity | 23 |
| Oropharynx | 39 |
| Larynx | 30 |
| Hypopharynx | 9 |
| TNM stage, % | |
| I | 11 |
| II | 5 |
| III | 11 |
| IV | 73 |
| Disease status, % | |
| No evidence of disease | 57 |
| Alive with disease | 5 |
| Dead of disease | 34 |
| Dead of unrelated causes | 5 |
| HPV status, % | |
| HPV16 positive | 30 |
| HPV16 negative | 70 |
| Normal samples (n = 25) | |
| age | 29 ± 12 |
| Sex, % | |
| Male | 36 |
| Female | 64 |
| Race, % | |
| Caucasian | 56 |
| African american | 44 |
| Smoking status, % | |
| Smokers | 12 |
| non-smokers | 88 |
| Drinking status, % | |
| Drink | 36 |
| Do not drink | 64 |

mRNA Extraction for Exon Array

Total cellular RNA was isolated using Trizol (Life Technologies, Gaithersburg, Md.) and RNeasy Kit (Qiagen, Valencia, Calif.) according to manufacturer's instructions, Oligonucleotide microarray analysis was carried out using Affymetrix GeneChip (Affymetrix, Santa Clara, Calif.) Human Exon 1.0 ST Array. Signal intensity and statistical significance was established for each transcript and normalized for COPA analysis.

### DNA Extraction for Methylation Array

Tissue samples were incubated in a solution of Sodium dodecylsulphate and proteinase K, for removal of proteins bound to DNA. DNA was purified by phenol-chloroform extraction. The DNA was subsequently resuspended in EDTA 2.5 mM and Tris-HCl 10 mM, pH 7.5 and submitted for array.

### Bisulfite Treatment and Sequencing

2 μg of DNA from 32 HNSCC tumors and 16 normal mucosa samples was subjected to bisulfite treatment using the EpiTect® Bisulfite Kit (Qiagen, Valencia, Calif.) according to the manufacturer's instructions. This bisulfite-treated DNA was then stored at −80° C. Subsequently, bisulfite treated DNA was amplified using primers designed by Methprimer to span areas of CpG islands upstream or in the promoter region. Genomic sequences for 36 genes were acquired from USCS browser. MethPrimer identifies CpG islands on criteria like GC content of >50%, >100 bp, >0.6 observed to expected CG's. Primer sequences were designed to not contain the CG dinucleotides. Touch down PCR was performed and products were PCR-purified using the QIAquick PCR Purification Kit (Qiagen, Valencia, Calif.), according to manufacturer's instructions. Each amplified sample was sequenced by Genewiz Inc., Germantown, Md.

### Quantitative RT-PCR

Total RNA was extracted as described and concentration for each sample was measured. 1 μg of RNA for qRT-PCR was reverse transcribed using qScript cDNA mix (Quanta Biosciences). Real time PCR was performed using Taqman Universal PCR Master mix on the ABI 7900HT real time PCR machine (Applied Biosystems).

### Cancer Outlier Profile Analysis (COPA)

Heterogenous patterns of proto-oncogene activation have been detected in many types of cancer and traditional approaches like t-tests and signal-to-noise ratios may fail to define significant alterations in expression for specific genes in high-throughput array approaches. COPA was applied to the total of 69 tissue samples, i.e. 44 tumor and 25 normal tissues with each gene expression data set containing 1.4 million probes and about 40 probes per gene. Gene expression values are centered, setting each gene's median to zero. The median absolute deviation is calculated and scaled to 1 by dividing each gene expression value by its MAD, hence giving transformed values that are preserved post-normalization. The 90th percentile of the transformed expression values were calculated for each gene and then genes were rank-ordered by their percentile scores, giving a systematic list of outliers. Traditional COPA methods are designed to find genes that are overexpressed in a subset of samples which would not permit identification of tumor suppressors. Hence, the one-sided COPA was manipulated in a unique fashion to a two-sided COPA so that tumor suppressors with masked expression could be identified. The two-sided COPA analysis method was applied to the gene methylation data set containing 27,578 CG dinucleotides spanning 14,495 genes as a novel strategy to find methylation outliers in a subset of samples.

### Cell Lines, Plasmids and Transfections

Head and Neck cancer cell lines were obtained from American Type Cell Culture (ATCC). Head and Neck cancer cell lines O22, O28, and O29 were cultured in RPMI1640 medium supplemented with 10% fetal bovine serum and 1% penicillin streptomycin. Immortalized Oral keratinocytes line OKF6 was cultured in keratinocyte serum free medium (Lonza, Allendale). All cell lines were harvested for DNA/RNA after growing for 48 hours. Cells were collected in QIAzol (Qiagen, Valencia Calif.) for total RNA extraction.

### Example 2

### Identification of Differentially Expressed Genes and Epigenetically Altered Genes Using a High-Throughput Approach

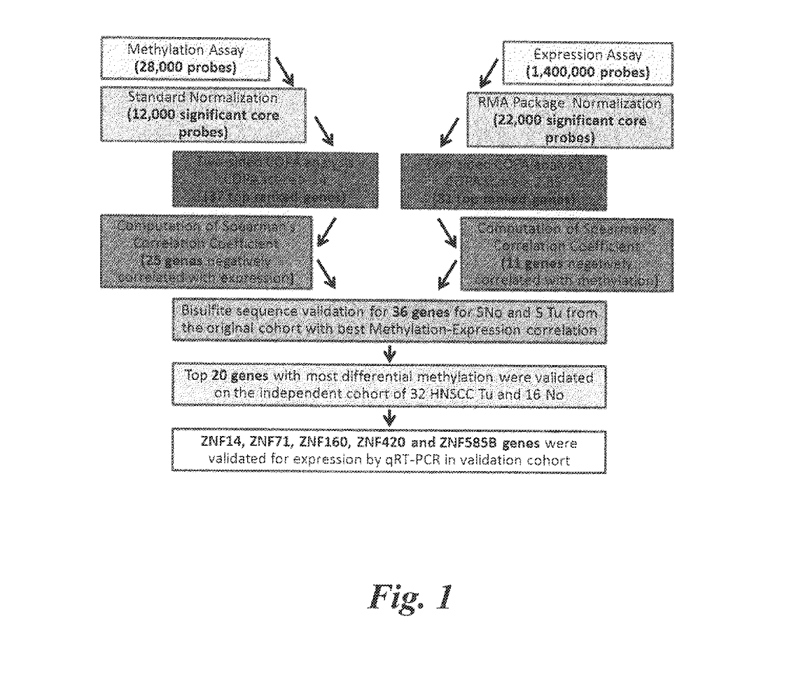

An integrative and high throughput approach was devised (FIG. 1) to identify genes that are candidate proto-oncogenes and tumor suppressors based on the hypothesis that such genes are transcriptionally activated and repressed in association with gene-specific promoter methylation alterations and (1) can be identified using genome wide integrative discovery techniques, and (2) can alter biologic pathways in a coordinated fashion. The first phase of the screening strategy involved expression and methylation profiling of 44 Head and Neck Squamous cell carcinomas (primary HNSCC) and 25 normal mucosal samples using the Affymetrix GeneChip Human Exon 1.0 ST array that contains 1,4 million probes and the Human Methylation 27 Microarray probing 27,578 CpG dinucleotides spanning 14,495 genes. All HNSCC tumor samples were selected to reflect a balance of site, sub site stage, and patient characteristics for analysis, ensuring that all patients have at least three years of clinical follow up. The 25 normal mucosal samples were collected from individual non-cancer patients.

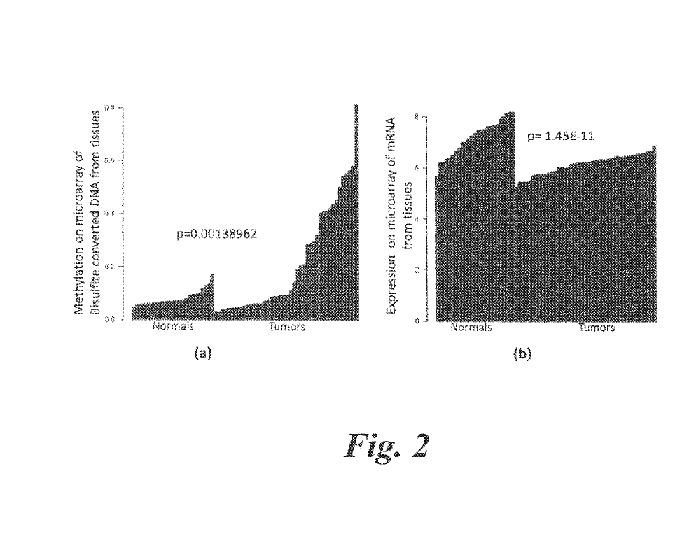

Raw data from the arrays were normalized using the R Oligo package which summarizes the data after background minimization and normalization. After normalization of expression array data sets, 22,000 core probe sets were deemed significant. Also, 12,000 core probe sets remained after normalization from the Methylation array data sets. For analysis of these remaining expression array and methylation array data sets, COPA was applied. A ranked list of 81 outlier genes was obtained using a COPA score cutoff of 2.3, showing differential expression above this threshold for tumors with lack of outlier expression in normal samples, i.e. hypothesized proto-oncogenes or vice versa for hypothesized tumor suppressors. Similarly a rank-ordered list of 37 outlier genes with a COPA score threshold of 14.4 was gained that had hypo methylated origins in HNSCC (candidate proto-oncogenes) or hyper methylated in HNSCC (candidate tumor suppressors). Methylation and Expression COPA graphs for a single gene, MAP4K1, a MEK kinase kinase are shown in FIGS. 2a and 2b, respectively. To examine the relation between differential methylation and expression in HNSCC, correlation analysis was conducted thereby integrating data from both arrays.

### Example 3

### Identification and Validation of Candidate Genes

The next objective was to integrate expression array data in primary HNSCC analyzed by COPA with data derived from COPA analysis of CpG methylation values determined using DNA methylation arrays. This objective served the ultimate aim to identify genes that are demethylated and overexpressed in primary HNSCC (putative proto-oncogenes) and methylated and underexpressed in primary HNSCC (putative tumor suppressor gene,). Accordingly, Spearman's correlation was computed for the 81 expression outliers and 37 methylation outliers. Without being bound to any one particular theory, it is believed that strong negative correlation signifies strong regulation of expression by methylation of CG dinucleotides in the promoter region of the gene in question. It also is believed that, ideally, the higher the methylation in the promoter of the gene, the more repressed is the expression of the gene and vice versa. Eleven expression outliers that negatively correlated with methylation were selected. Similarly, 25 methylation outliers that negatively correlated with expression also were chosen. Therefore, a total of 36 candidate genes were identified by integration of data from both arrays through correlation analysis and these genes were selected to be validated by bisulfite sequencing. It also was desired to confirm that the array probes were representative of the methylation status of the entire promoter CpG islands for each individual gene.

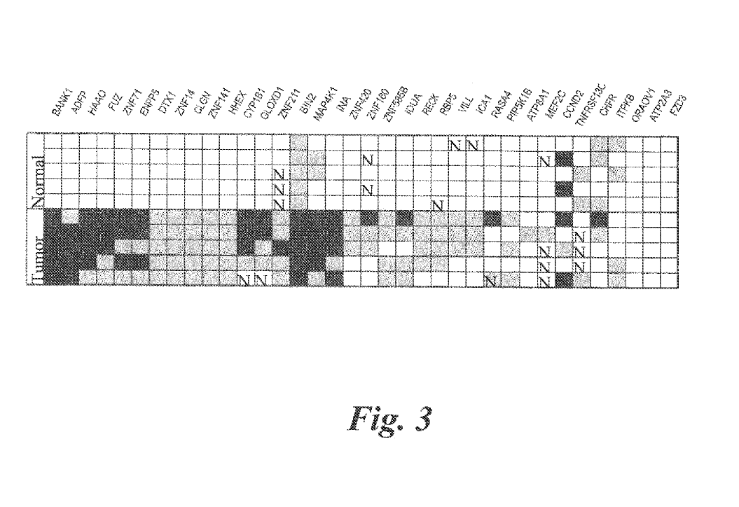

To validate the methylation array data, CpG islands in the promoter region of the 36 selected gene targets were bisulfite sequenced in five normal mucosal samples paired with five primary HNSCC tumor samples from the initial discovery cohort to confirm differences in methylation. Primer pairs were designed using Methprimer software and they were located upstream and around the promoter region. Sample pairs were chosen on the basis of highest difference in methylation and concurrent expression as computed during two sided COPA analysis for each individual gene. Of these 36 targets, 33 showed differential methylation as tumors were compared with normals (FIG. 3). Therefore, promoter gene methylation status was validated for 33 candidate genes out of 36 selected genes. Of the 36 targets, 26 candidates showed greater than 50% methylation or semi-methylation in tumor tissues, including BANK1, DTX1, MAP4K1, ZNF71, ZNF14, and the like.

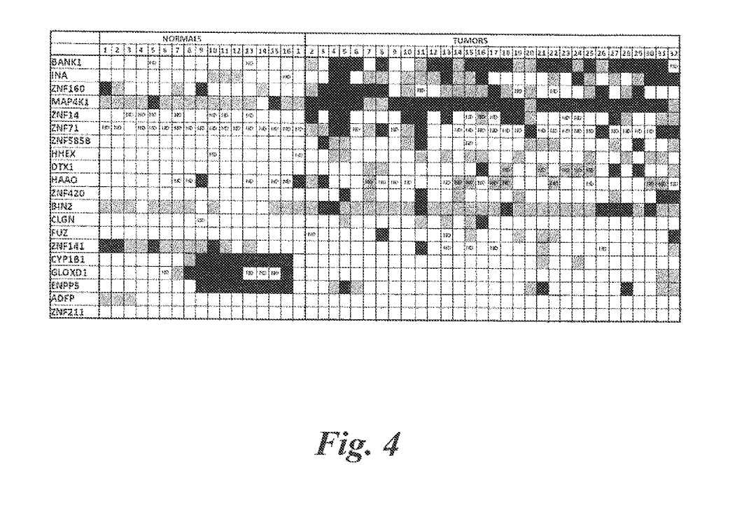

The 20 best biologically relevant candidates were chosen for validation in a separate cohort with characteristics similar to that of the discovery cohort. Bisulfite sequencing was performed on all candidate 20 genes in a cohort of 32 primary HNSCC tumor tissues and 16 normal mucosal samples. Out of the 20, 13 genes showed over 40% difference in methylation, including BANK1 wherein 81% of tumors were more methylated as compared to normal (FIG. 4). MAP4K1, a MEK kinase kinase showed 53% methylation difference between. normals and tumors. It was noted that from the top 13 genes, there were five zinc fingers that showed at least 40% difference or more in the methylation status of normals and tumors. Without wishing to be bound to any one particular theory, this data suggested that these zinc fingers are regulated epigenetically in a coordinated fashion.

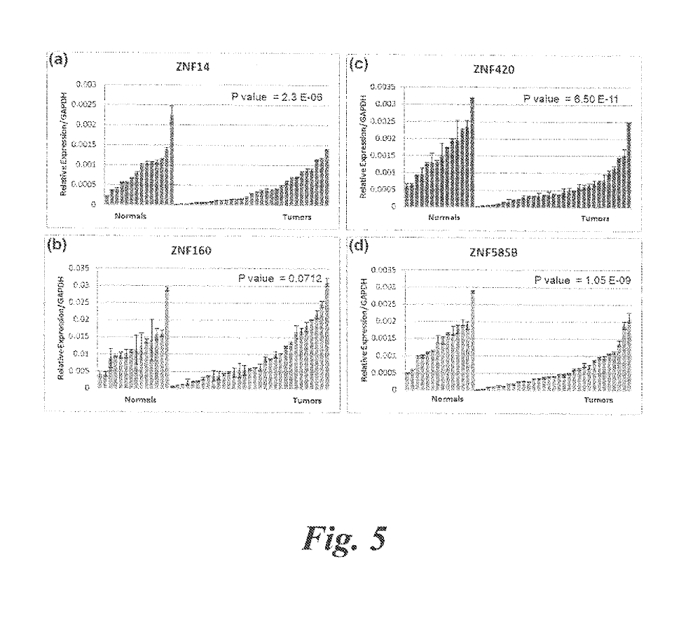

To determine the relationship between promoter methylation to expression, RT-PCR was subsequently performed for five zinc fingers including ZNF160, ZNF14, ZNF420, ZNF585B, ZNF71 for 32 tumors and 16 normals from the validation cohort. ZNF14 (FIG. 5a), ZNF160 (FIG. 5b), ZNF420 (FIG. 5c), ZNF585B (FIG. 5d), showed a significant difference in expression profiles while comparing normal with primary tissues. Expression was significantly higher in normal mucosal samples for all four candidates whereas expression was higher in primary tumors for ZNF71 (Data not shown). Hence, there is a significant association for the four mentioned zinc fingers between promoter methylation and expression. ZNFs expression is coordinately associated with demethylation of individual promoter CpG islands.

Expression and methylation profiles of different cell lines were further analyzed to confirm the findings in vitro. Bisulfite sequencing was performed on the promoter regions of the candidate genes for common head and neck cancer cell lines including O22, O28, O29 and normal oral keratinocyte cell line i.e. OKF6. It was found that 22A, O28, O29 had a similar methylation status as the primary tissues in both cohorts (data not shown). Among the normal coil lines, OKF6 gene promoter was consistently found as unmethylated which makes it a good model to serve as a control. However, to ensure that the cell lines are similar to the primary tissues in every respect, quantitative RT-PCR was conducted to confirm expression and methylation correlation. There was no expression of ZNF420 in all three cancer cell lines, which was most likely due to the heavily methylated promoter CpG islands as determined by the bisulfite sequencing. It was concluded that ZNF's expression is coordinately associated with methylation status of individual promoter CpG islands in Human Head and Neck Cell lines O28, O29, O22 and OKF6 (data not shown). Therefore, target ZNFs expression is coordinately regulated by methylation in HNSCC Cell lines.

### Example 4

### Summary and Discussion

The presently disclosed subject matter provides a novel integrative screening strategy to specifically look for coordinately expressed genes in human HNSCC whose transcription is driven by promoter demethylation.

44 primary HNSCC and 25 normal mucosal samples were used in the Affymetrix GeneChip Human Exon 1.0 array and for the Illumina Infinium Human Methylation 27 array. To analyze the data, a novel screening approach based on Cancer Outlier Profile Analysis (COPA) was used. 81 significant differentially expressed and 37 significant differentially methylated genes with both oncogenic and tumor suppressing properties were determined using this approach. Out of the total 118 candidate genes, 36 candidate tumor suppressing genes with strong correlation between hypermethylation and decrease of expression in tumor samples were identified. 20 genes out of a total of 36 were validated by bisulfite sequencing in a separate cohort of 32 primary HNSCC tumor and 16 normal mucosal tissues. Out of the total 20 genes, 13 demonstrated at least 40% difference in methylation. Seven of these newly discovered genes out of the 36 belong to the Zinc Finger Proteins (ZNF) group and they have never been reported as tumor suppressors in HNSCC before. Bisulfite sequencing and quantitative real time PCR (qRT-PCR) analysis of ZNFs revealed that they are strongly hypermethylated and underexpressed in HNSCC tumor samples. This new screening approach allowed identification of 36 candidate tumor suppressor genes aberrantly methylated and transcriptionally suppressed in HNSCC. Validation of gene expression and methylation allowed the detection of ZNF genes whose downregulation may play a significant role in HNSCC tumor development.

BANK1ADFP, HAAO, FUZ, ZNF71, ENPP5, DTX1, ZNF14, CLGN, ZNF141, HHEX, CYP1B1, GLOXD1, ZNF211, BIN2, MAP4K1, INA, IDUA, RECK, ZNF585B, RBP5, VILL, ZNF420, ZNF160, RASA4, PIP5K1B, ATP8A1, MEF2C are candidate tumor suppressor genes of HNSCC that were found to be specifically hypermethylated in tumor samples. The data suggest that these are prospective tumor suppressor genes and the majority of them were never described to play a role in HNSCC. Hypermethylation of 14 of them, BANK1, INA, ZNF160, MAP4K1, ZNF14, ZNF71, ZNF585B, HHEX, DTX1, HAAO, ZNF420, BIN2, CLGN, FUZ was successfully demonstrated on the independent cohort of HNSCC, suggesting the importance of these genes in HNSCC carcinogenesis.

Expression of ZNF14, ZNF160, ZNF420, and ZNF585B was to be dramatically affected by the methylation in tumor samples, however the detailed functional studies revealed that exogenous manipulations of their expression levels correlated with oncogenenic properties of these ZNF proteins, suggesting that their increased methylation and consequent decrease in expression in tumor samples is a result of pathogenetic changes in HNSCC samples. The extent of the detection of the promoter methylation of ZNF14, ZNF160 and ZNF420 in the salivary rinse of HNSCC and healthy patients was analyzed. DNA methylation of at least one ZNFs promoter was detected in the salivary rinses of HNSCC patients with sensitivity of 22% (95% CI: 12.3%-34.7N and specificity of 100% (95% CI: 89.9%400%). Overall frequency and detection concordance of DNA methylation from at least one ZNFs promoter in. salivary rinse with signal found in primary tissues was 35.3% and 92.3%, respectively, ZNF methylation was strongly associated with oral cavity SCC (p=0.0049).

A potential use of the presently disclosed subject matter, along with others, is to target BANK1, ADFP, HAAO, FUZ, ZNF71, ENPP5, DTX1, ZNF14, CLGN, ZNF141, HHEX, CYP1B1, GLOXD1, ZNF211, BIN2 , MAP4K1 , INA, IDUA, RECK, ZNF585B, RBP5, VILL, ZNF420, ICA1, ZNF160, RASA4, PIP5K1B, ATP8A1 and/or MEF2C with therapeutic agents.

Another potential use of the presently disclosed subject matter may be the use of promoter hypermethylation of the three ZNF genes, ZNF14, ZNF160 and ZNF420, as salivary rinse gene or regulatory regions for HNSCC incidence and recurrence, especially among high-risk group population.

In summary, four genes, ZNF160, ZNF14, ZNF420, and ZNF585B, were identified that showed both differential expression and promoter region hypermethylation in HNSCC. These four genes are known transcription regulators and have not been associated with Head and Neck squamous cell carcinomas previously. Interestingly, each of these zinc finger proteins have a conserved Kruppel associated box domain (KRAB-A) that is a transcription repression module. Proteins containing a KRAB-A domain play important roles in cell differentiation and organ development, and in regulating viral replication and transcription. Also, all four of them are located on Chromosome 19. In fact, ZNF420 and ZNF585B are located at the same locus, 19q.12. A parallel study was conducted using the same primary HNSCC and normal mucosal samples to determine copy number on the Affymetrix Genome-wide SNP 6.0 Array containing 950,000 copy number probes. The copy number was checked for all four genes to ensure that there were no amplification or deletions and no significant difference was found in copy number for all four genes. It is more likely that this region is epigenetically silenced during HNSCC progression or there is an unknown common mechanism for epigenetic regulation.

Three other zinc fingers, ZNF71, ZNF211, ZNF141 were in the top 20 hits; however ZNF71 unsuccessfully showed negative correlation between expression and methylation during validation of expression by quantitative RT-PCR and it would be reasonable to assume that ZNF71 expression is regulated by means other than promoter CpG island methylation, most likely copy number changes, insertions, deletions, post transcriptional modification as well as post translational modifications etc. The other two genes did not meet the criteria due to failure to show complete methylation of promoter regions in the validation cohort by bisulfite sequencing and it is possible that, by using less rigorous standards, these and other genes that were differentially expressed would be able to be identified. In this study, only the top 20 of the 36 possible targets identified were selected during the intermediate validation step for further analysis. Further investigation of the remaining 16 genes may allow for recognition of additional novel epigenetically controlled genes and serve as targets for screening of gene or regulatory regions and therapeutic agents.

Out of the 20 genes chosen for validation in a separate cohort by bisulfite sequencing, seven other genes showed more than 40% methylation status difference between normal mucosal samples and primary HNSCC namely BANK1, INA, MAP4K1, HHEX, DTX1, BIN2 and HAAO. Two other genes, CLGN and FUZ, showed less than 40% difference in methylation, which could be improved by using a larger sample size.

The ZNFs described in this study have not been previously associated with HNSCC progression. ZNF420 more commonly known as Apak is an established regulator of p53 and hence a main player in stress related apoptosis during DNA damage. ZNF160 is a known transcriptional repressor of TLR4, which contributes to amyloid peptide-induced microglial toxicity. Although the function of KRAB-ZFPs is largely unknown, they appear to play important roles during cell differentiation and development. Using the head and neck cancer cell lines described in this study that model primary tissues, further studies may be performed for determining function and biological significance in tumor progression. The primary application of this work is the production of epigenome-wide information for the identification and characterization of therapeutic targets and predictive gene or regulatory regions. Four novel potential tumor suppressor zinc fingers have been described herein that have coordinated repression of expression by methylation of individual promoter regions in primary HNSCC.

### Example 5

### Further Materials and Methods

### Tissue Samples

Three independent cohorts of HNSCC patient specimens and normal specimen controls have been used. The discovery cohort included 44 primary HNSCC tissues and 25 normal samples from uvulopalatopharyngoplasty (UPPP). The first validation cohort included 32 primary HNSCC tissues and 15 normal UPPP samples. The second validation cohort included primary tumor samples and salivary rinse samples from 59 HNSCC patients, 31 normal UPPP samples and 35 normal salivary rinse samples. All samples were obtained from the Head and Neck Tissue Bank at johns Hopkins, acquired under Hopkins Internal Review Board approved research protocol #NA_{—}00036235. All primary tissue and body fluid specimens were stored at —140° C. (liquid nitrogen) until use. All primary tissue samples were analyzed by the Pathology Department at Johns Hopkins Hospital. Tumor samples were confirmed to be HNSCC and subsequently microdissected to separate tumor from stromal cells to yield at least 75% tumor cells. The characteristics of three cohorts are listed in Tables 2, 3 and 4.

### DNA Preparation

Microdissected tissue samples or 250 μl aliquots of bodily fluid samples were digested in 1% SDS (Sigma) and 50 μg/ml proteinase K (Invitrogen) solution at 48° C. for 48-72. hours for removal of proteins bound to DNA. DNA was then purified by phenol-chloroform extraction and ethanol precipitation as previously described (Shao et al., 2012). DNA was resuspended in LoTE buffer (EDTA 2.5 mM and Tris-HCl 10 mM, pH 7.5), and DNA concentration was quantified using the NanoDrop ND-1000 spectrophotometer (Thermo Scientific).

### RNA Preparation

RNA was isolated from the microdissected tissue samples with the use of mirVana miRNA Isolation Kit (Ambion) per manufacturer's recommendations, and RNA concentration was quantified using the NanoDrop.

### Arrays

Ten micrograms of RNA and DNA were submitted to the Johns Hopkins Core Facility for Quality Control query and analysis by high throughput arrays. Samples were run on Affymetrix HuEx1.0 GeneChips for expression analysis (with over 1.4 million probe coverage) and Illumina Infinium HumanMethylation27 BeadChips for methylation analysis (28 thousand probe coverage) following bisulfite conversion. All arrays were run according to manufacturer protocols.

### Statistical Analysis

Significant Core Probes Determination. Gene expression data was normalized with RMA with the Bioconductor oligo package (Carvalho and Irizarry, 2010; Gentleman et 2004); 22 thousand core probes were generated. For promoter methylation data, custom R scripts were used to generate approximately 12 thousand probes with three or more CpG islands. Gene level estimates were produced by choosing the highest mean expression levels and highest mean methylation levels among all probes linked to the same gene for expression and methylation respectively. This yielded expression estimates for 16330 genes, and methylation estimates for 8676 genes.

Two-sided Cancer Outlier Profile Analysis (COPA). COPA was applied to the total of 69 tissue samples from the discovery cohort with each gene expression data set containing 22 thousand probes and methylation data set containing 12 thousand probes. The median absolute deviation is calculated and scaled to 1 by dividing each gene expression value by its MAD, hence giving transformed values that are preserved post-normalization (MacDonald and Ghosh, 2006). COPA analysis was applied to both gene expression and methylation data sets as a novel strategy to find both expression and methylation outliers in a subset of samples. COPA scores were calculated for both upper-tail (90^{th} percentile) and lower-tail (10th percentile) cases; this allowed definition of outliers that are overexpressed, downregulated, hypermethylated and hypomethylated. COPA score cut-off for expression data was set on 2.35 to give approximately top 100 genes; COPA score cut-off for methylation data was set on 13.2 to give approximately top 50 genes (Table 5).

Evaluation of ZNF methylation signals in primary tissues and matched saliva samples for the patients from the second validation cohort. P-values calculations and univariate analysis. Hypermethylation of each gene was treated as a binary variable (methylation vs. no methylation) by dichotomizing the methylation at zero. Factors tested for prognostic value included the age, sex, race, smoking status, alcohol use, HPV status, primary tumor site, pathologic tumor stage, pathologic nodal stage, clinical TNM stage, and the presence of promoter methylation of ZNF14, ZNF160 and ZNF420. Overall survival was defined as the time elapsed from the date of completion of therapy to the date of death from any cause or the date of last follow-up. Proportional hazards models were used to assess the univariate prognostic significance of clinical variables and each individual methylation marker on overall survival. The Hazard Ratios (HRs) were calculated relative to a reference group and presented with their corresponding 95% CIs. All reported P-values for testing the differences between groups were based on Fisher exact test, or Wilcoxon test upon property. P value less than 0.05 was considered statistically significant.

### Reverse Transcription and Quantitative Real Time PCR

1 μg of RNA from the first validation cohort was reverse transcribed using the High Capacity cDNA Reverse Transcription Kit (Applied Biosystems, Carlsbad, Calif., USA). Quantitative real-time PCR was performed using gene-specific expression assays (Table 9) and Universal PCR Master mix on the 7900HT real time PCR machine (all from Applied Biosystems). PCR conditions were 1 cycle; 95° C. for 10 min; followed by 40 cycles: 95° C. for 15 s and 60° C. for 60 s. Expression of the gene of interest was quantified relative to GAPDH expression using the 2-ΔΔCT method (Livak and Schmittgen, 2001).

TABLE 9
Primers, Probes and Expression Assays Used in the Presently Disclosed Subject Matter

| Applied | | | |
|---|---|---|---|
| Biosystems- | | | |
| recommended | | | |
| expression | | | |
| assays | | | |
| No | Gene name | Assay ID | |
| 1 | ZNF14 | Hs00221420_m1 | |
| 2 | ZNF71 | Hs01934418_s1 | |
| 3 | ZNF160 | Hs00369142_m1 | |
| 4 | ZNF420 | Hs01557830_m1 | |
| 5 | ZNF585B | Hs04189951_m1 | |
| 6 | GAPDH | Hs02758991_g1 | |
| Quantitative | | | |
| Methylation- | | | |
| specific PCR | | | |
| (QMSP) | | | |
| primers and | | | |
| probes | | | |
| No | Gene name | Foward Primer | Reverse Primer |
| 1 | ZNF14 | GGATATCGTGATTTTTCGGACGTTG | CGACTACGAATCCAACTCCCACAA |
| 2 | ZNF160 | GAAATCGTTTGAAATATTTACGTCGTT | AACGAAACTAAACGAAACACGTTA |
| 3 | ZNF420 | GGTATGGTGTTCGGAGCGTT | CACGCGAAACCTCCAAATCT |
| 4 | β-actin | TGGTGATGGAGGAGGTTTAGTAAGT | AACCAATAAAACCTACTCCTCCCTTAA |
| No | Gene name | Probe | |
| 1 | ZNF14 | 6FAM/AAACCGAACTACGCCCGCGATAACC/TAMRA | |
| 2 | ZNF160 | 6FAM/ACGATTTCGTATAATACCCACAACCCAACGCT/TAMRA | |
| 3 | ZNF420 | 6FAM/TAGAGGTATCGTTTTCGGAGCGTAGT/TAMRA | |
| 4 | β-actin | 6FAM/ACCACCACCCAACACACAATAACAAACACA/TAMRA | |
| No | Gene name | Foward Primer | Reverse Primer |
| Bisulfite | | | |
| sequencing | | | |
| PCR primers | | | |
| 1 | ADFP | GATTTTAGGTAGGGTTATTTTTATTTTTA | CCAAACAAACCAAAAAACATTC |
| 2 | ATP2A3 | TTGGTTATGTGAGGAATAATTTTT | CCCATTCTACAAAAAAAAAAACTAAAAC |
| 3 | ATP8A1 | GAGTATTATGGGTATTAGGGGTTTTT | ACTCTCTCACATCTTACTCAAAAAAAA |
| 4 | BANK1 | TGAGTAGTTTTATTTTTTTTGGG | AAAAAAACCCTCTAAACTACCTAAC |
| 5 | BIN2 | GGTTTAGAGTTTATTTGGAGTAAGAAA | TCAATAATAAACCCACACTCACTC |
| 6 | CCND2 | GGGTTGGTTATGGAGTTGTTG | AACATCCAAATAACCACCATTCTAC |
| 7 | CHFR | GGATTTGTGTGATTTATTGTGTGTAAT | ACCATCTTTAATCCTAACCAAAC |
| 8 | CLGN | GATTTGTAGGGGGAATTTTTTTT | AAAACCCAATCAAAACCCTAACT |
| 9 | CYP1B1 | ATGAAAGTTTGTTGGTAGAGTTT | CTAAACACCTACTACCCTCACTA |
| 10 | DTX1 | TTGGAAATAAAGATGATAAAGATTTAAGT | AAAATAAAATCCCTAAACACCC |
| 11 | ENPP5 | GGGGGTAATTAGGTAGAAGTGATTAT | AATTATATTCCCAATTTCCCAATCAT |
| 12 | FUZ | GGTTTTTTGGTTTTTTTTATTTTTT | TCCAAAACCCCACCTACTAAC |
| Applied | | | |
| Biosystems- | | | |
| reccommended | | | |
| expression | | | |
| assays | | | |
| 13 | CZD3 | GGGTTTATTTTTGTTTGTTTA | AAACACCCTTAACTTCTCTTACAACAA |
| 14 | GLOXD1 | GTAGTTATTGTGAGTTTTTGGGTTG | ACCTAAACTTATCCTTCTAAAACC |
| 15 | HAAO | TTTTTAGATGGGAAAGTTAAATTTTGA | AAAAAATCCAAACCCTTCCTAAAC |
| 16 | HHEX | | |
| 17 | ICA1 | GGGTTGTAGGAAGTAGTAGGAGA | CTTATCAACAAATCAACCCTAAAC |
| 18 | IDUA | GTTTTATTTAGGAGGTTGGGGTG | CAAAAACCTATACTCCTCCAAAAAC |
| 19 | INA | GGTGGGTGTAGGGGATATTTT | AAACTCCTACTCAAAATCTAACC |
| 20 | ITPKB | GGTTGTTTTGGATAGTTAATGTTTGTT | CCTACAAAACCCAAAAAAAAACC |
| 21 | MAP4K1 | AGGTGTTAGAAGTTGAGTTTTGAGG | ATCAAAAACTAAAACCCCCTCTTAC |
| 22 | MEF2C | AAGAGTGAAATTGATGATTTTTTTAGTT | ATACTTCTCCACCTAATTCAAACATACA |
| 23 | ORAOV1 | TTTTAAAGTGTTGGGATGATAGG | CCCAAAACAACCTATACATAAC |
| 24 | PIP5K1B | GGGGTTTGTAGTTTTTTTAGT | CAACAAAAATACAAAACCCCTAAAC |
| 25 | RASA4 | TTGAGATAGAAGAATTGTTTGAAAT | TCCTAAAAAACAATACCCCTCC |
| 26 | RBP5 | TGGGGAGAAAGAAGTTAGAAGTTAG | CCTCCTTAAATCCCAAAACCT |
| 27 | RECK | TTGAGGTTTTGGTTTGTTATTTAT | AAAACAAAAATTTCTCTCCTCAAAC |
| 28 | TNFRSF13C | GAGGGTTGAAAGGATTTTGTG | CTTCTCTCCCCCTCAAAAAC |
| 29 | VILL | TTTGGGGAAGTTTGTTTGAGA | ACTTACCCCATTCAAAAATATAAAC |
| 30 | ZNF14 | GTTATTGGATTTGTTTAATTAGGA | AAATTAACTACAAAAAAATCCCC |
| 31 | ZNF141 | GAGTTTGGGGAGGGAGATATATTT | TCCTCACAAAACCTAATTAAATACACA |
| 32 | ZNF160 | AGAGGAAAGTAGTTTGGTTTTTAAAATAAT | AACAAAAACCCCAAAAAAAA |
| 33 | ZNF211 | TGAAAATTTAAGATAGGGGTATTTT | CTCTCACTTAAAACTTAAAAATCTC |
| 34 | ZNF420 | GGGATAAGTAGGTTTTATAGGT | AAAATCCAAAATCTAACTCCC |
| 35 | ZNF585B | TGGGTTGAAATTGGTTTTTAAGT | TAACTAACCCTACAAACCCTCAATC |
| 36 | ZNF71 | GTTTTTTGTGAGATGGAGGAGTTTA | CTACCTATCTCTCACACAAACCAC |

### Bisulfite Treatment and Bisulfite Genomic Sequencing

The EpiTect Bisulfite Kit (Qiagen, Valencia, Calif.) was used to convert unmethylated cytosines in genomic DNA to uracil (Gaykalova et al., 2012), according to the manufacturer's instructions. Converted DNA was stored at −80° C. until use. Subsequently, bisulfite-treated DNA was amplified with primers designed using MethPrimer to span areas of CpG island(s) (Li and Dahiya, 2002). Primer sequences were specifically designed to contain no CG dinucleotides (Table 9). Touch-down PCR was performed as follows: 95° C. for 5 min, followed by 43 cycles consisting of a 30 s denaturation step at 95° C., 30 s at an annealing temperature, 1 min at the initial extension at 72° C., with a final extension for 5 min at 72° C. The annealing temperature was gradually decreased, e.g. two cycles at each of 64° C., 62° C., 60° C., 58° C. and then 35 cycles at 56° C. (Shao et al., 2011). The PCR products were purified using the QIAquick 96 PCR Purification Kit (Qiagen, Valencia, Calif.), according to the manufacturer's instructions. Purified PCR products were then subjected to the direct sequencing (Genewiz Inc., Germantown, Md.).

### Quantitative Methylation-Specific PCR (QMSP)

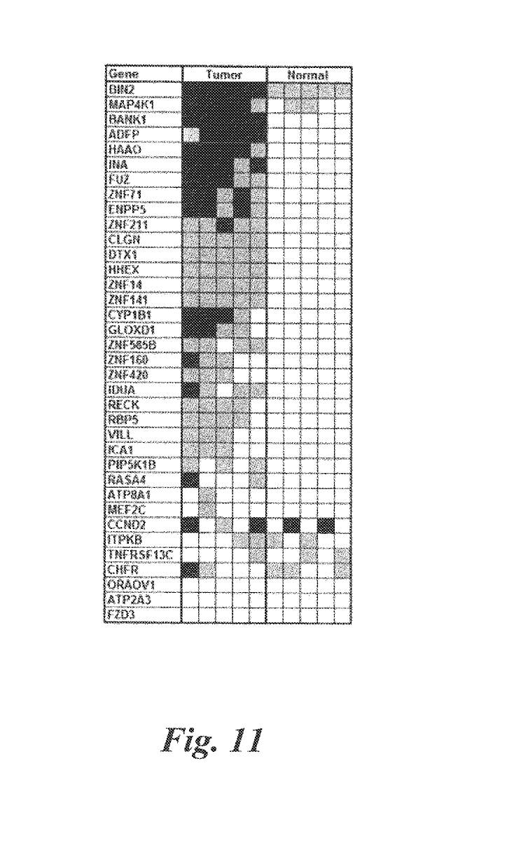

For QMSP, primers were designed to specifically include CpG dinucleotides that showed changes in methylation seen by bisulfite sequencing (FIG. 11). QMSP was performed using Platinum Tag DNA Polymerase (Invitrogen) on the 7900HT real-time PCR machine with normalization to unmethylated β-actin internal reference control, for which primers were designed to avoid CpGs in the sequence (Bhan et al., 2011; Kim et 2006; Shao et al., 2011). Bisulfite Converted Universal Methylated Human DNA Standard (Zymo Research) was used in serial dilutions (50-0.005 ng) to construct a calibration curve for each plate. All samples were within the range of sensitivity and reproducibility of the assay based on the amplification of the internal reference control. Bisulfite converted leukocyte DNA from a healthy individual was used as a negative control. The relative level of methylated DNA in each sample was determined as a ratio of qMSP-amplified gene to β-actin (Kim et al., 2006), multiplied by 100 for easier tabulation. Sequences of the primers and probes used can be found in Table 9.

### HPV Analysis

Available pathology reports have been obtained regarding the HPV status of oropharyngeal HNSCC tumors that have been tested in clinic by in situ hybridization (ISH) for high-risk HPV and p16 IHC staining (Singhi et al., 2012). In addition to this, the HPV status of all oropharyngeal HNSCC primary tissues was independently confirmed by quantitative PCR (qPCR) using HPV16 primers and probe on the 7900HT real-time PCR machine as described (Carvalho et al., 2011). In short, specific primers and probes have been used to amplify the E6 and E7 regions of HPV 16, and normalized the data to housekeeping gene (β-actin). The genomic DNA from CaSki cell line (American Type Culture Collection, ATCC, Manassas, Va.), known to have 600 copies of HPV16 per genome (6.6 pg of DNA/genome), was used in serial dilutions (50-0.005 ng) to construct a calibration curve for β-actin, HPV 16 E6 and E7 for each plate. The relative level of HPV16 DNA in each sample was determined as a mean of ratios of E6 and E7 amplified gene to β-actin, multiplied by 300, that gave number of copies per genome per tumor cell. HPV copy number >1 copy/genome/cell was regarded as HPV positive.

### Cell Culture

Cell Lines and Cell Culture Conditions. Human head and neck squamous cell carcinoma (HNSCC) cell lines JHU-011, JHU-022, JHU-028 and JHU-029 were developed from primary HNSCCs in the Division of Head and Neck Cancer Research (Johns Hopkins University, Baltimore, Md.); UM-SSC-22A and UM-SSC-22B were obtained from Dr. Thomas E. Carey (University of Michigan, Ann Arbor, Mich.); FADU was obtained from ATCC (Rocco et al., 1998; Zhao et al., 2011). OKF6 cells are a minimally transformed oral keratinocyte line was donated by Dr. James Rheinwald (Harvard University, Cambridge Mass.). NOK-SI cells are normal oral keratinocytes that spontaneously immortalized and were provided by Dr. Silvio Gutkind (National Institutes of Health, Bethesda, Md.) (Hennessey et al., 2011), Cell lines 011, 022, 028, 029 and FADU were cultured in RPMI1640 medium supplemented with 10% fetal bovine serum and 1% penicillin streptomycin (Corning), 22A and 22B were cultured in DMEM with 4.5 μg/ml glucose medium supplemented with 10% fetal bovine serum and 1% penicillin streptomycin (Corning). OKF6 and NOKSI cell lines were cultured in keratinocyte serum free medium (Lonza, Walkersville, Md.). Cell growth conditions were maintained at 37° C. in an atmosphere of 5% carbon dioxide and 95% relative humidity. Cell line DNA and RNA was extracted as described above for primary tissues.

Transient Transfection and Cell Proliferation Assay. ZNF14, ZNF160 and ZNF420 expression plasmids with control empty vector (pCMV6-AC-GFP); and small hairpin RNA (shRNA) plasmids for ZNF14, ZNF160 and ZNF420 knockdown with control scrambled shRNA plasmid were purchased from Origene (Rockville, Md.). Cells were seeded in 96-well plates and allowed to grow in recommended medium until the cells were approximately 70% confluent. Cells were transfected with a ZNF expressing and control empty vector or with ZNF shRNA and scrambled shRNA plasmids using Fugene HP (Roche, Indianapolis, Ind.). Cell metabolic activity was determined every 24 hours using the CCK-8 colorimetric assay (Dojindo, Gaitherburg, Md.) at 450 nm according to the manufacturer's instructions. Values are mean±SEM for pentaplicates of cultured cells.

### Example 6

### HNSCC Patients and Control Population Characteristics for the Initial Discovery

The first discovery cohort of the study population consisted of 44 patients with a historically confirmed diagnosis of HNSCC that received the conventional surgery from November 1999 through January 2010 and 25 non-cancerous patients that received uvulopalatopharyngoplasty (UPPP) from September 2008 through January 2010. The Characteristics of the study population largely reflect the demographics of head and neck cancer patients in the United States (Table 2). The HNSCC patients were mainly males (73%, 32 of 44) and Caucasians (91%, 40 of 44), with ages ranging from 45 to 80 years (median±SD=58±13 years), Smoking and alcohol consumption was found in 61% (27 of 44) and 57% (25 of 44) of all patients, respectively, with average packs per year of 39.7±30.3, With regard to HPV status, the study population consisted of 30% (13 of 44) HPV-positive patients. The primary tumor was located in the oral cavity (23%, 10 of 44), oropharynx (39%, 17 of 44). hypopharynx (9%, 4 of 44), or larynx (30%, 13 of 44). 73% of the patients (32 of 44) presented with locally advanced stage IV disease. The median fellow up time of these patients was 31.4 months (range; 0.5-117.3 months). At the end of the follow-up period, 10 patients were alive with the disease. During the follow up period, 14 (32%) recurrences were detected, including 8 local recurrences. As of January 2013, a total of 21 patients (48%) have died. The cause of death was head and neck cancer in 18 out of 21. patients, with the other 3 patients dyed of the other causes. The control population was mainly female (64%, 16 of 25) and Caucasian (56%, 14 of 25), with ages ranging from 18 to 65 (29±12 years). Smoking and alcohol consumption was found in 12% (3 of 25) and 36% (9 of 25) of all patients, respectively, with average packs per year of 29.0±293.

TABLE 2
Clinical characteristics of recruited HNSCC
patients for the initial discovery

| | | | Normal | |
|---|---|---|---|---|
| | HNSCC | | samples | |
| | (n = 44) | | (n = 25) | |
| | n (%) | | n (%) | |
| Median age (range) | 58 ± 13 | (45-80) | 29 ± 12 | (18-65) |
| Male | 32 | (73%) | 9 | (36%) |
| Female | 12 | (27%) | 16 | (64%) |
| Race | | | | |
| Caucasian | 40 | (91%) | 14 | (56%) |
| African American | 3 | (7%) | 11 | (44%) |
| Others | 1 | (2%) | | |
| Smoking status | | | | |
| Packs per year (range) | 39.7 ± 30.3 | (4-125) | 29.0 ± 29.7 | (8-50) |
| Smokers | 27 | (61%) | 3 | (12%) |
| Non-smokers | 12 | (28%) | 22 | (88%) |
| Unknown | 5 | (11%) | | |
| Drinking status | | | | |
| Drink | 25 | (57%) | 9 | (36%) |
| Do not drink | 12 | (27%) | 16 | (64%) |
| Unknown | 7 | (16%) | | |
| HPV16 positive | 13 | (30%) | | |
| Tumor site | | | | |
| Oral cavity | 10 | (23%) | | |
| Oropharynx | 17 | (38%) | | |
| Larynx | 13 | (30%) | | |
| Hypopharynx | 4 | (9%) | | |
| TNM stage | | | | |
| I | 5 | (11%) | | |
| II | 2 | (5%) | | |
| III | 5 | (11%) | | |
| IV | 32 | (73%) | | |
| Disease status | | | | |
| No evidence of disease | 22 | (50%) | | |
| Alive with disease | 1 | (2%) | | |
| Dead of disease | 18 | (41%) | | |
| Dead of unrelated causes | 3 | (7%) | | |

### Example 7

### Identification of Differentially Expressed Genes and Epigenetically Altered Genes Using a High-Throughput Approach

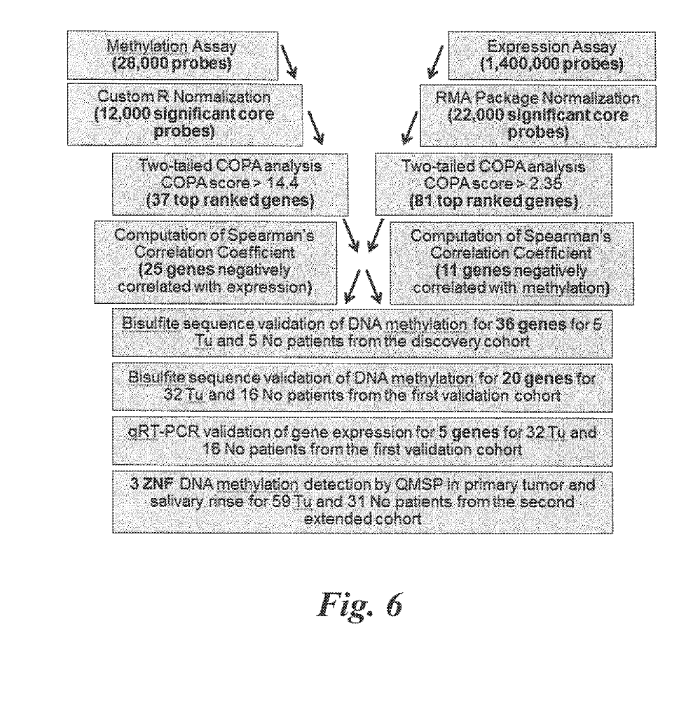

An integrative statistical approach was devised using high-throughput data from expression and methylation approach and applying the COPA method (FIG. 6) to identify proto-oncogenes and candidate tumor suppressor genes related to HNSCC carcinogenesis. The approach was based on the hypothesis that changes in the gene-specific expression are associated with gene promoter methylation alterations. The first phase of the screening strategy involved high-throughput expression and methylation profiling of 44 HNSCC and 25 normal mucosal samples. Modern Affymetrix GeneChip Human Exon 1.0 ST array which contains 1.4 million probes and the Human Methylation 27 Microarray probing 27,578 CpG dinucleotides were used. Raw data from the arrays were normalized and background noise was eliminated using the RMA and R Oligo packages. After normalization of the expression array data sets, 22,000 core probe sets were deemed significant. Similarly, 12,000 core probe sets remained after normalization from the methylation array data sets.

For analysis of these remaining expression array and methylation array probe sets, COPA was applied. COPA is a test based on robust centering and scaling of data to detect outlier samples (MacDonald and Ghosh, 2006). It is better adapted to the outliers that are characteristic of cancer-related biologic alterations, where traditional t-test and signal-to-noise approaches may fail due to the low rate of cancer-related events. The upper-tail and lower-tail COPA scores for the expression and methylation arrays were then combined, alloying the identification of 118 candidate genes (Table 5). 81 of them were selected from the expression array data received after the cut-off COPA score was set at 2.35 to provide a list of approximately 100 top-scoring candidates for both prospective oncogenes (90th percentile COPA) and tumor suppressor genes (10th percentile COPA). Similarly, a list of 37 outliers genes with COPA score threshold of 13.2 for the methylation array data set was gained. This threshold was set in order to provide approximately 50 top-scoring genes both hypomethylated (10th percentile COPA for prospective oncogenes) and hypermethylated (90th percentile COPA for prospective tumor-suppressor genes).

TABLE 5
COPA scores for 118 candidates from
Expression and Methylation arrays

| | | |
|---|---|---|
| No | Gene name | COPA score |

Expression assay candidates (n = 81)

| | | |
|---|---|---|
| 1 | MSMB | 10.63639872 |
| 2 | P2RX5 | 7.153518011 |
| 3 | CRISP3 | 6.656476129 |
| 4 | MAGEA4 | 6.312319093 |
| 5 | SPIB | 5.699751061 |
| 6 | SYCP2 | 5.335378956 |
| 7 | BANK1 | 4.965466419 |
| 8 | TLR10 | 4.785272414 |
| 9 | N/A | 4.599501155 |
| 10 | CR2 | 4.54443077 |
| 11 | SLCO1B3 | 4.54147024 |
| 12 | CXCR5 | 4.340322133 |
| 13 | C1orf110 | 4.101499784 |
| 14 | ORAOV1 | 4.000264983 |
| 15 | SPINK6 | 3.918116862 |
| 16 | STATH | 3.895714578 |
| 17 | SCIN | 3.841408015 |
| 18 | LRMP | 3.761357411 |
| 19 | RASSF9 | 3.759748747 |
| 20 | ZNF382 | 3.723258863 |
| 21 | NTS | 3.676033141 |
| 22 | AMTN | 3.536749249 |
| 23 | PDE5A | 3.511170705 |
| 24 | CD72 | 3.494882884 |
| 25 | PLCG2 | 3.456105326 |
| 26 | DTX1 | 3.400217622 |
| 27 | CALB1 | 3.38630449 |
| 28 | PARP15 | 3.353755323 |
| 29 | RGS13 | 3.287563003 |
| 30 | KRT78 | 3.268367973 |
| 31 | TMPRSS11B | 3.250814009 |
| 32 | PAX5 | 3.224598904 |
| 33 | TREM1 | 3.208419329 |
| 34 | GAPT | 3.180415777 |
| 35 | CD79B | 3.161574048 |
| 36 | KLRB1 | 3.159680114 |
| 37 | C13orf18 | 3.139616396 |
| 38 | TNFRSF13C | 3.115543283 |
| 39 | BTK | 3.045869938 |
| 40 | FZD3 | 3.01547693 |
| 41 | SASH3 | 2.985848116 |
| 42 | 38960 | 2.985521065 |
| 43 | NRCAM | 2.983959908 |
| 44 | CD22 | 2.953511254 |
| 45 | BIN2 | 2.940241578 |
| 46 | SFRP4 | 2.932011756 |
| 47 | GRAP | 2.931051639 |
| 48 | OGN | 2.879218969 |
| 49 | STAR | 2.870796993 |
| 50 | CD180 | 2.820108977 |
| 51 | ARHGAP15 | 2.771944678 |
| 52 | LY86 | 2.726672406 |
| 53 | SIT1 | 2.726227992 |
| 54 | N/A | 2.72415117 |
| 55 | FERMT3 | 2.707636854 |
| 56 | CD22 | 2.703099769 |
| 57 | XRCC2 | 2.701465418 |
| 58 | INA | 2.693294975 |
| 59 | VAV1 | 2.643280369 |
| 60 | ATP8A1 | 2.598980447 |
| 61 | DSC1 | 2.594995898 |
| 62 | ACTC1 | 2.57712684 |
| 63 | MAP4K1 | 2.575268072 |
| 64 | CD19 | 2.532865786 |
| 65 | ARHGAP25 | 2.523625955 |
| 66 | CYP1B1 | 2.504898279 |
| 67 | GCET2 | 2.493075428 |
| 68 | N/A | 2.457970505 |
| 69 | CD69 | 2.456991995 |
| 70 | ATP2A3 | 2.450835826 |
| 71 | FCRL3 | 2.4477349 |
| 72 | WDFY4 | 2.431746698 |
| 73 | PADI1 | 2.431658487 |
| 74 | MCOLN2 | 2.418303706 |
| 75 | P2RY10 | 2.410328347 |
| 76 | CD274 | 2.397999095 |
| 77 | VNN2 | 2.395671888 |
| 78 | KRT1 | 2.385638566 |
| 79 | IL24 | 2.370364144 |
| 80 | SERPINA9 | 2.366812563 |
| 81 | AMIGO2 | 2.350831555 |

Methylation assay candidates (n = 37)

| | | |
|---|---|---|
| 1 | HHEX | 67.51054185 |
| 2 | VILL | 47.65082835 |
| 3 | CHFR | 37.23973321 |
| 4 | ZNF160 | 36.2084266 |
| 5 | MPDU1 | 31.13417209 |
| 6 | ZNF134 | 28.9373152 |
| 7 | BSG | 28.1162042 |
| 8 | FLJ22688 | 28.08415274 |
| 9 | CLSTN3 | 27.69596653 |
| 10 | RBP5 | 24.39698918 |
| 11 | MEF2C | 23.479971 |
| 12 | CLGN | 23.40885042 |
| 13 | IDUA | 21.7933596 |
| 14 | PIP5K1B | 20.67732627 |
| 15 | ADFP | 19.83258688 |
| 16 | ZNF420 | 19.46419102 |
| 17 | ZNF141 | 19.02108471 |
| 18 | RASA4 | 18.80958368 |
| 19 | KCNQ1 | 18.67906133 |
| 20 | RAB39 | 17.85805985 |
| 21 | ZNF211 | 17.77623482 |
| 22 | RHOF | 17.05440732 |
| 23 | ENPP5 | 16.47452634 |
| 24 | ZNF71 | 16.36372914 |
| 25 | CCND2 | 16.2578808 |
| 26 | GLOXD1 | 16.18834852 |
| 27 | ICA1 | 15.63623555 |
| 28 | ZNF14 | 15.4458021 |
| 29 | HAAO | 15.41014583 |
| 30 | SLC8A3 | 15.18526618 |
| 31 | RECK | 15.04356674 |
| 32 | ITPKB | 14.60792797 |
| 33 | PFKFB4 | 14.52909531 |
| 34 | ZNF585B | 14.41258458 |
| 35 | JAM2 | 13.49463275 |
| 36 | HIST1H3I | 13.47289421 |
| 37 | RHOF | 13.21077435 |

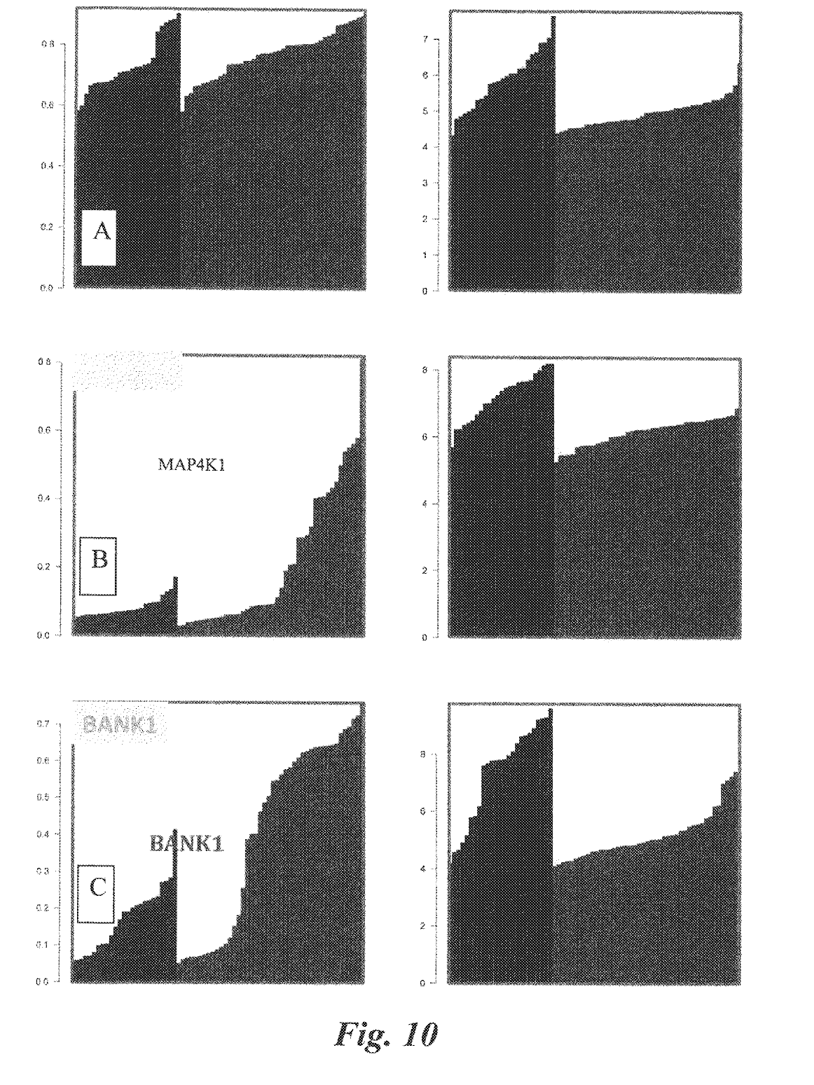

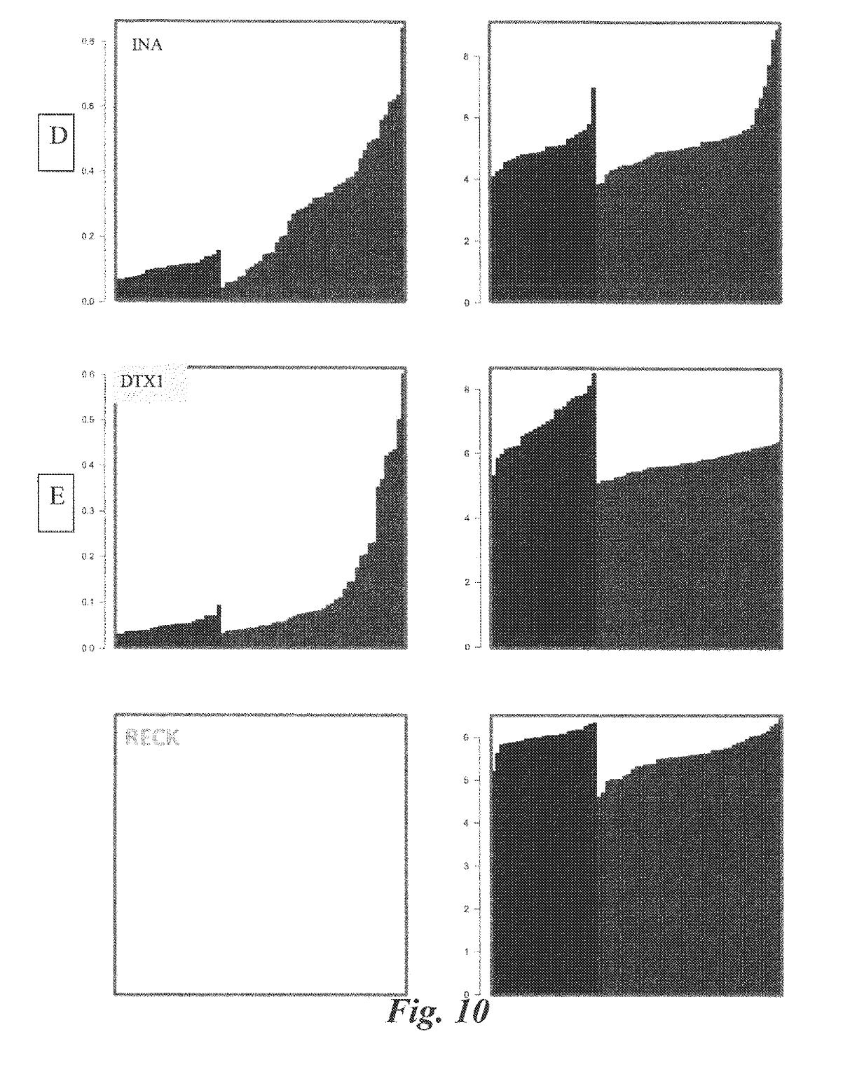

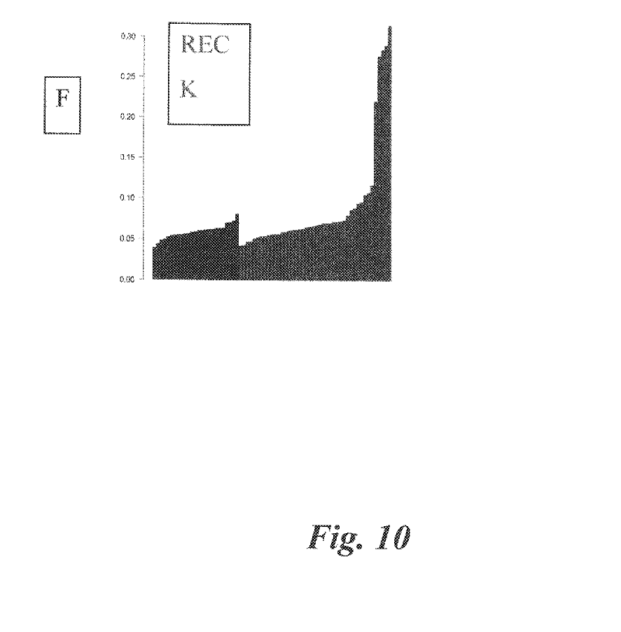

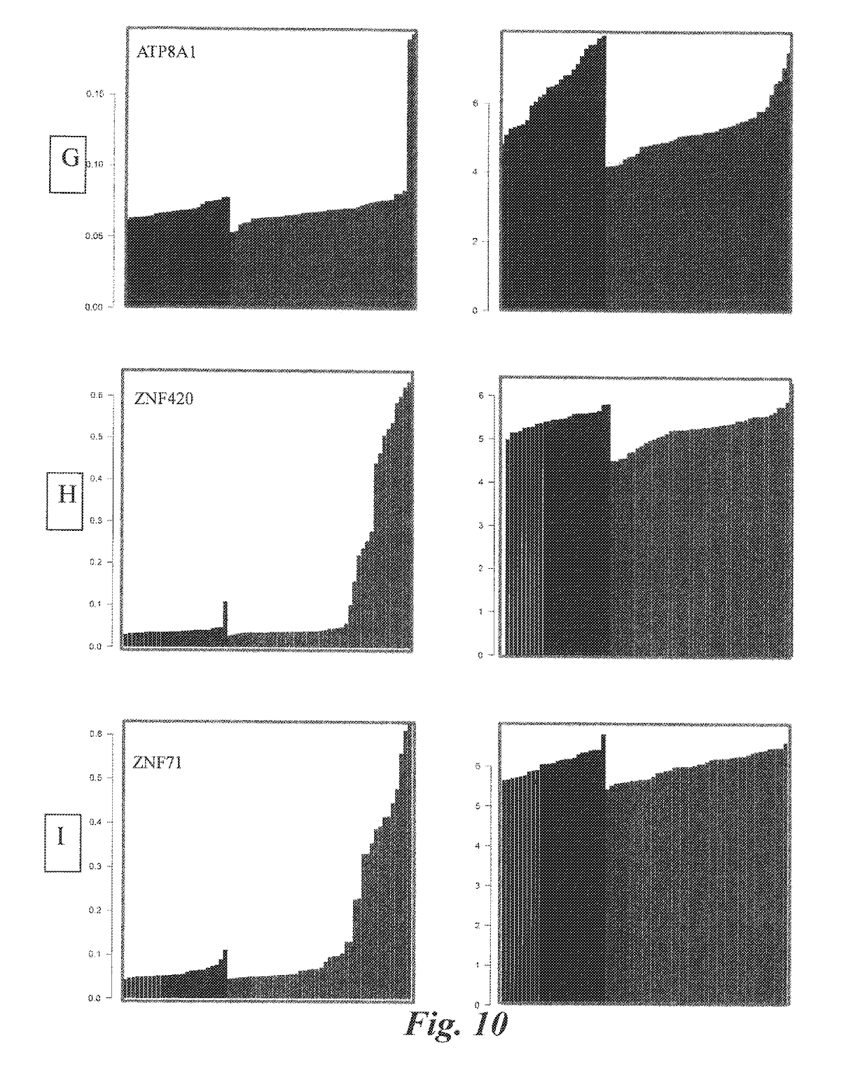

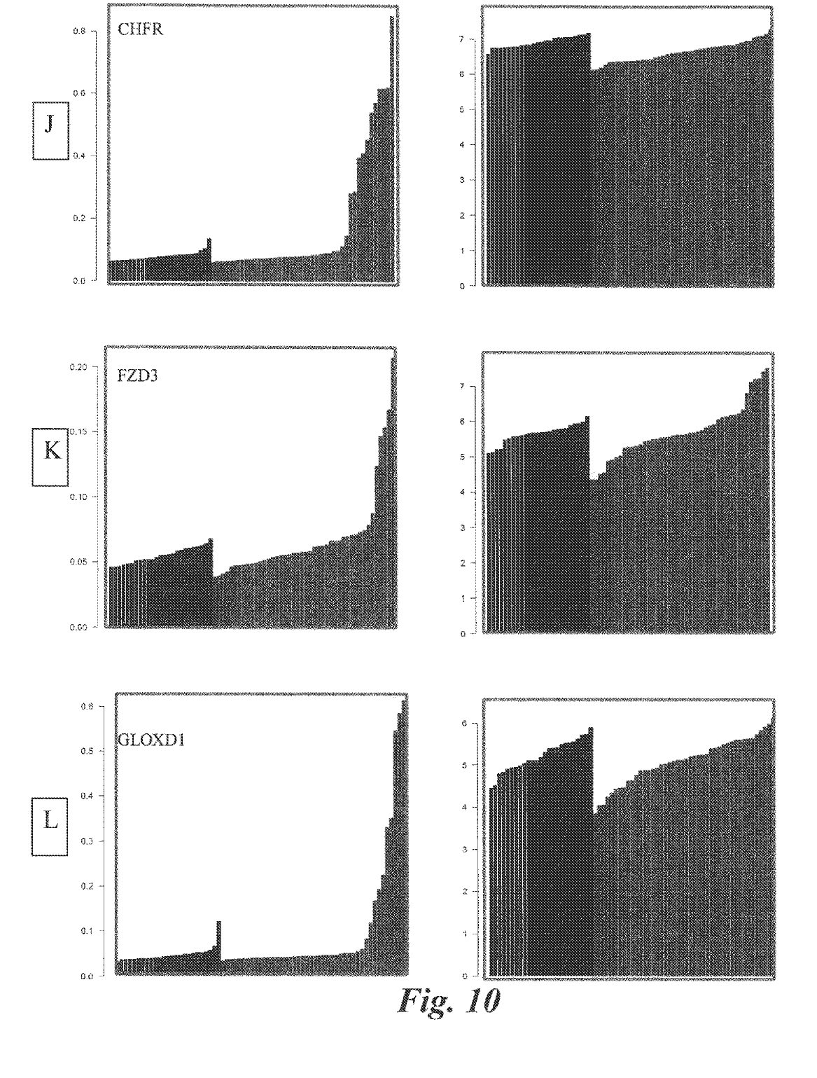

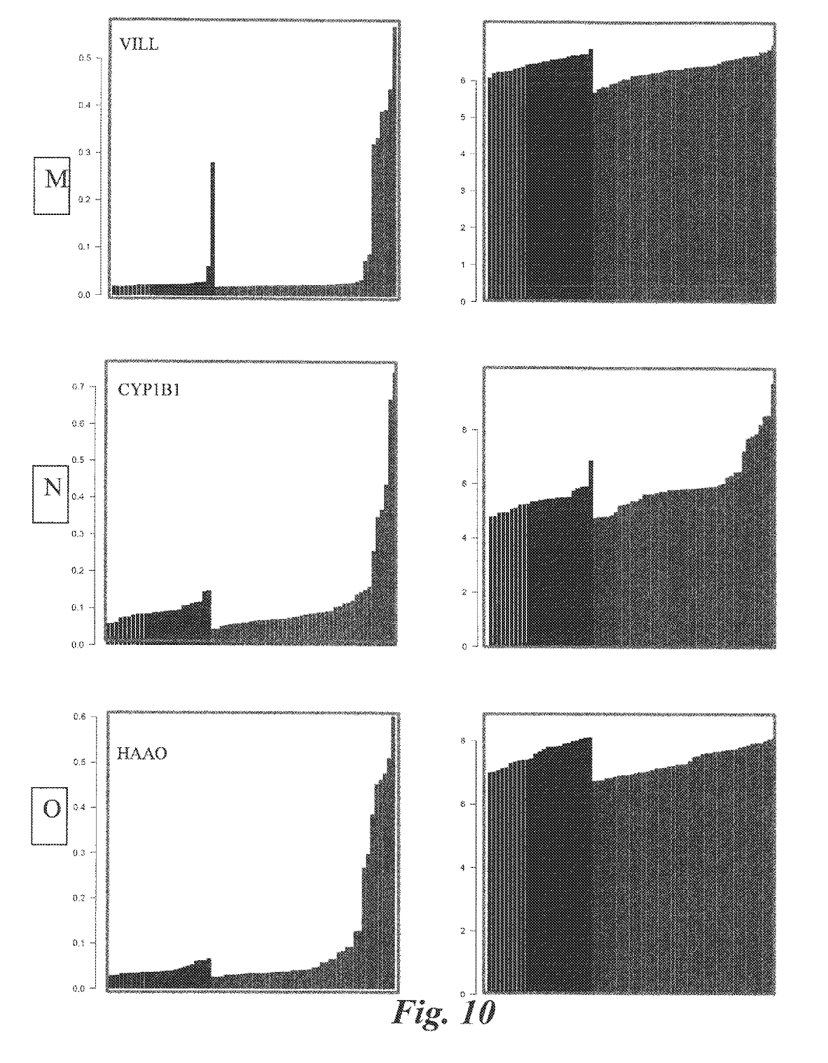

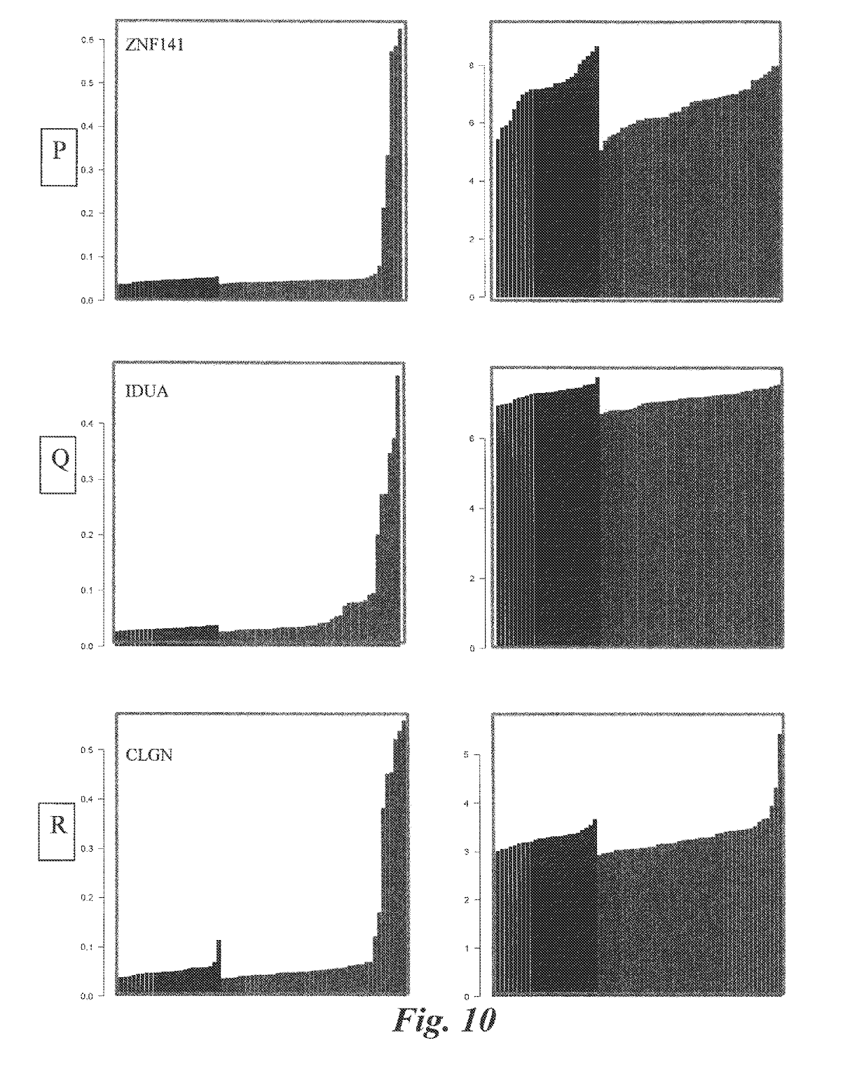

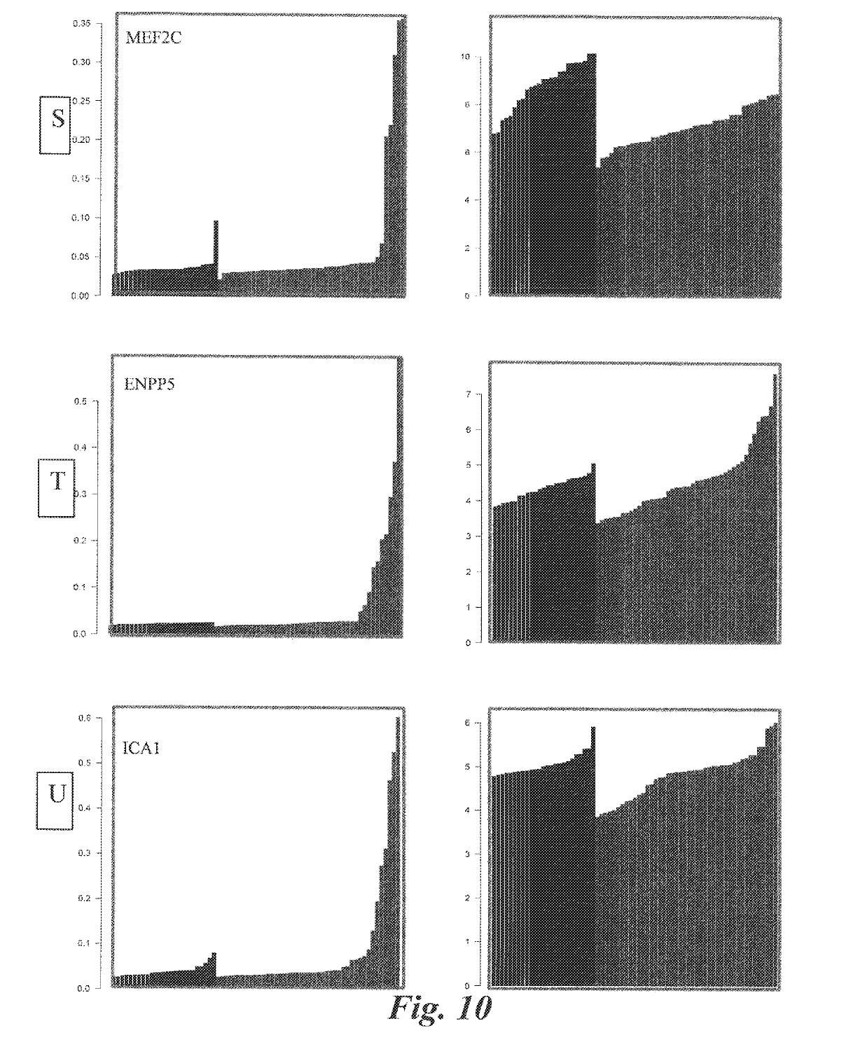

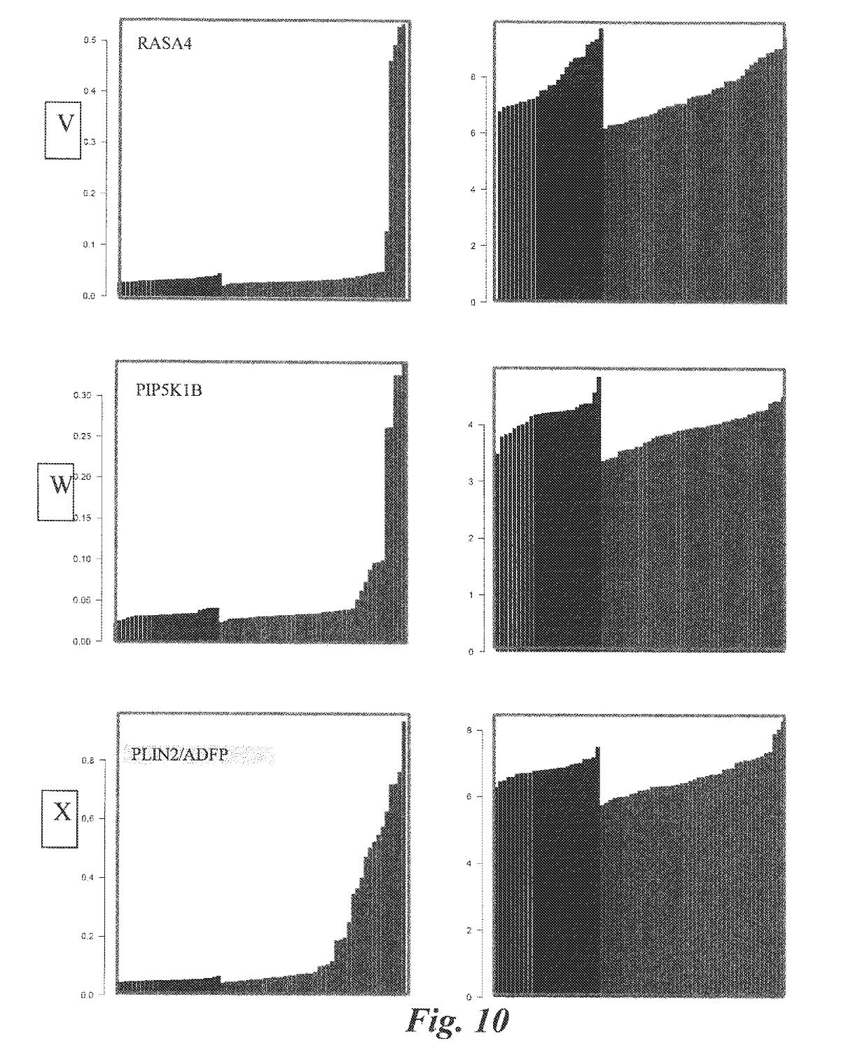

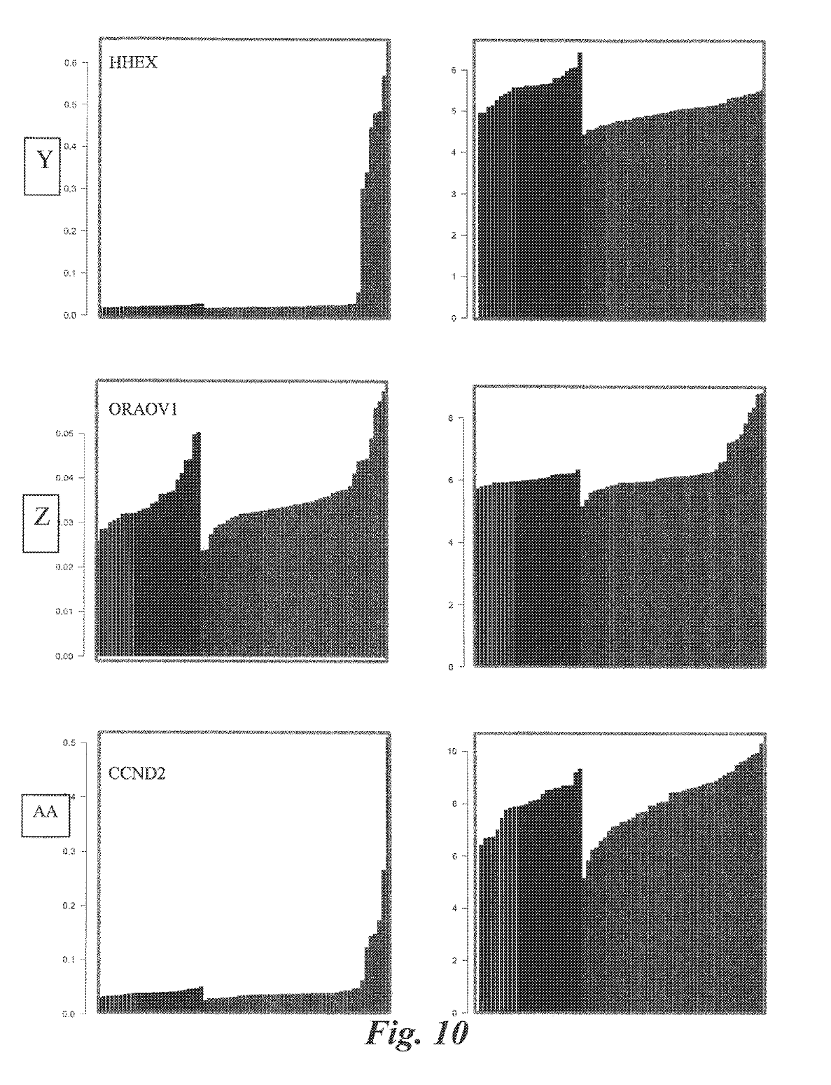

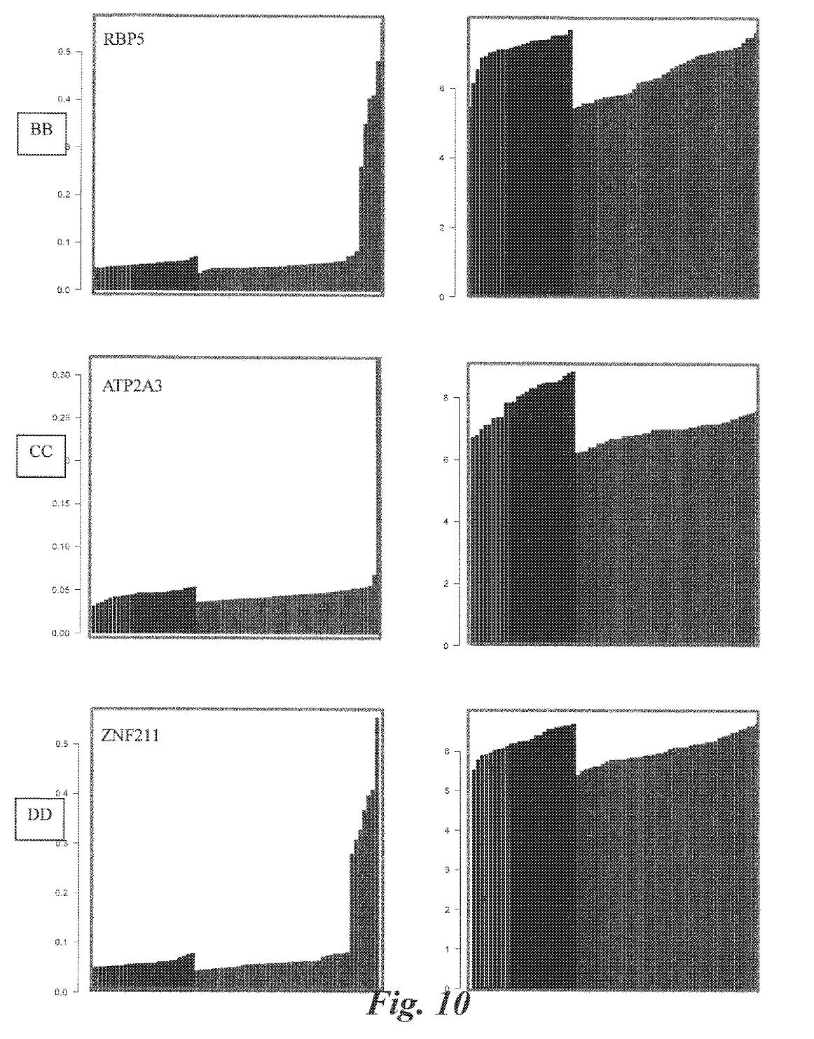

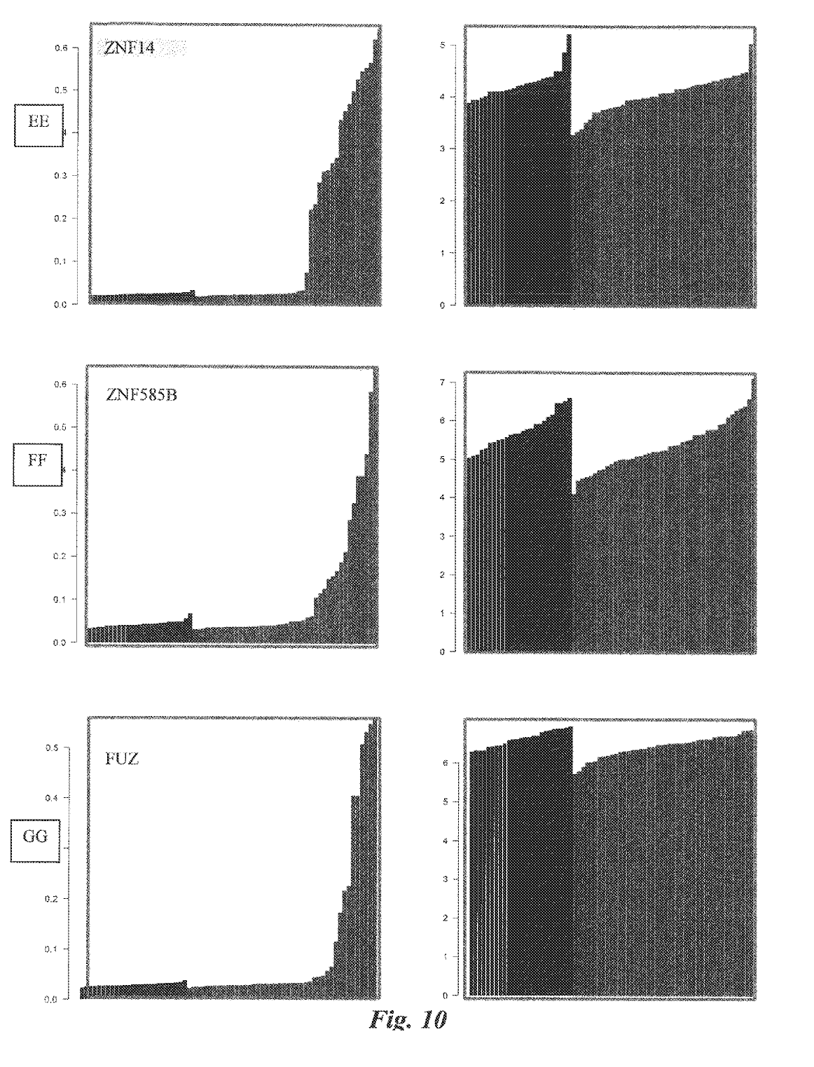

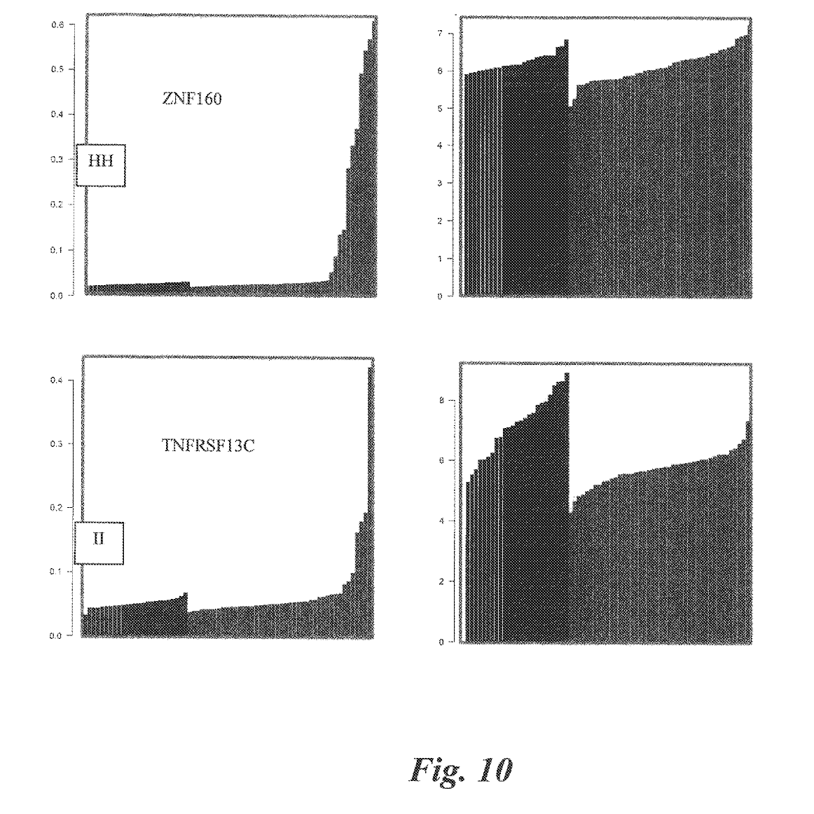

To evaluate the relationship between methylation and expression in HNSCC, Spearman's correlation was computed for the 81 expression outliers and 37 methylation outliers. The hypothesis was that strong negative correlation signifies strong regulation of expression by methylation of CpG dinucleotides in the promoter region of the gene. Due to the restrains of the methylation array, not all candidates from the expression array had evaluated methylation status. From the expression arrays, only 11 candidates had negatively correlated methylation. Similarly, 25 methylation array outliers that had negatively correlated with expression were chosen. The total 36 genes were used for the further validation (Table 6 and FIG. 10). It is important to note, that all 36 candidate genes had potential tumor-suppressing properties based on their expression and methylation status, and the majority of those genes had never been reported for HNSCC.

TABLE 6
Spearman Expression-Methylation correlation coefficient

| | | | Spearman |
|---|---|---|---|
| No | Gene name | Gene Function | coefficient |
| 1 | ADFP | Adipocyte | −0.220 |
| | | differentiation | |
| 2 | ATP2A3 | ATPase | −0.120 |
| 3 | ATP8A1 | ATPase | −0.195 |
| 4 | BANK1 | Scaffold protein | −0.420 |
| 5 | BIN2 | Bridging integrator | −0.693 |
| 6 | CCND2 | Cyclin D2 | −0.014 |
| 7 | CHFR | Checkpoint | −0.464 |
| 8 | CLGN | Chaperone protein | −0.135 |
| 9 | CYP1B1 | cytochrome | −0.18 |
| 10 | DTX1 | Notch-pthw regulator | −0.274 |
| 11 | ENPP5 | phosphatase | −0.150 |
| 12 | FUZ | Fuzzy homolog | −0.290 |
| 13 | FZD3 | Frizzled receptor | −0.161 |
| 14 | GLOXD1 | Dioxygenase-like | −0.16 |
| 15 | HAAO | dioxygenase | −0.072 |
| 16 | HHEX | Trascription factor | −0.079 |
| 17 | ICA1 | autoantigen | −0.327 |
| 18 | IDUA | iduronidase | −0.063 |
| 19 | INA | neurofilament | −0.333 |
| 20 | ITPKB | Inositol kinase | −0.090 |
| 21 | MAP4K1 | MAP kinase | −0.590 |
| 22 | MEF2C | enhancer | −0.137 |
| 23 | ORAOV1 | OC oncogene | −0.06 |
| 24 | PIP5K1B | kinase | −0.151 |
| 25 | RASA4 | RAS p21 activator | 0.002 |
| 26 | RBP5 | Retinol binding | −0.123 |
| 27 | RECK | MMP9 regulator | −0.222 |
| 28 | TNFRSF13C | TNF receptor | −0.30 |
| 29 | VILL | Villin-Iike | −0.235 |
| 30 | ZNF14 | Zinc finger | −0.234 |
| 31 | ZNF141 | Zinc finger | −0.355 |
| 32 | ZNF160 | Zinc finger | −0.440 |
| 33 | ZNF211 | Zinc finger | −0.177 |
| 34 | ZNF420 | Zinc finger | −0.280 |
| 35 | ZNF585B | Zinc finger | −0.396 |
| 36 | ZNF71 | Zinc finger | −0.058 |

### Example 8

### Promoter Hypermethylation Validation of Candidate Tumor-Suppressor Genes

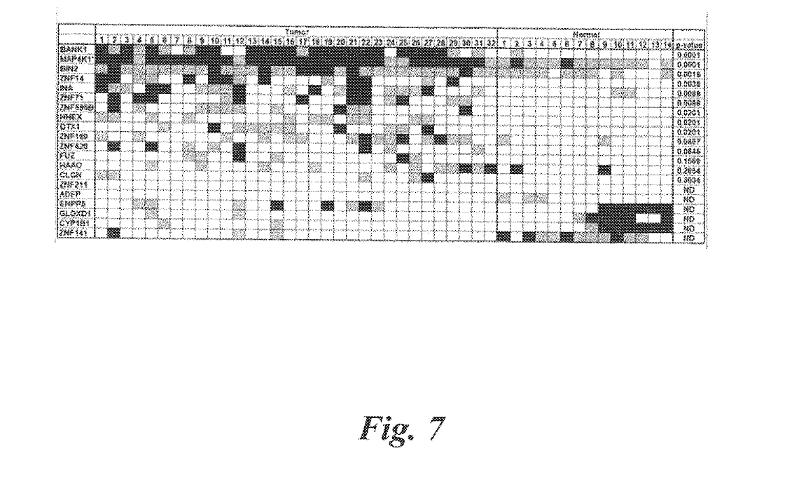

To validate the differential methylation status of the CpG islands near the promoter region of the 36 selected candidate genes, bisulfite sequencing of 5 normal mucosal samples and 5 primary HNSCC tumor samples from the initial discovery cohort was performed. Primer pairs were designed using MethPrimer software (Li and Dahiya, 2002) and located within the CpG island around the promoter region with close proximity to the methylation array probes. Sample pairs were chosen on the basis of highest difference in methylation and concurrent expression as computed during COPA analysis for each individual gene. Bisulfite sequencing was chosen at this step in order to obtained the absolute (not relative or normalized) data about the methylation status of the several CpG dinucleotides in the CpG island near the promoter of each gene. Gene methylation status was determined as a trichotomous variable (unmethylated, semimethylated and hypermethylated, FIG. 11). Of these 36 genes, 31 showed differential methylation as tumors were compared with normal samples (FIG. 11). 26 candidates (72%) showed greater than 50% methylation in tumor tissues, including BANK1, DTX1, MAP4KI, ZNF7I, ZNE14 etc. Twenty top-scoring biologically relevant candidates were chosen for validation in a separate validation cohort composed of 32 HNSCC tumor tissues and 15 normal mucosal samples with demographic and clinical characteristics similar to that of the discovery cohort (Table 3). Fourteen out of the twenty genes (70%), including BANK1, INA, MAP4KI, and five different ZNF proteins, showed significant difference in methylation (FIG. 7). Out of the five ZNF protein genes ZNF14, ZNF160, ZNF420 and ZNF585B belong to Kruppel-associated box (KRAB)-containing ZNF proteins, while ZNF71. does not. KRAB box is a transcription repression module; that fact supports the hypothesis that those ZNF proteins are prospective tumor suppressor genes. All but ZNF14 ZNF are located on the 19q13 locus, which was shown to be epigenetically silenced in oropharyngeal cancer (Lleras et al., 2011), which supports the hypothesis that those ZNF are regulated epigenetically in a coordinated fashion.

TABLE 3
Clinical characteristics of recruited HNSCC
patients from the first validation cohort

| | HNSCC | | Normal samples | |
|---|---|---|---|---|
| | (n = 32) | | (n = 14) | |
| | n (%) | | n (%) | |
| Median age (range) | 62 ± 11 | (41-87) | 33 ± 12 | (18-57) |
| Male | 24 | (75%) | 5 | (64%) |
| Female | 8 | (25%) | 9 | (36%) |
| Race | | | | |
| Caucasian | 27 | (84%) | 8 | (57%) |
| African American | 4 | (13%) | 6 | (43%) |
| Others | 1 | (3%) | | |
| Smoking status | | | | |
| Packs per year | 600 ± 700 | (105-1095) | 13.4 ± 14.8 | (3-36.5) |
| (range) | | | | |
| Smokers | 22 | (69%) | 7 | (50%) |
| Non-smokers | 8 | (25%) | 7 | (50%) |
| Unknown | 2 | (6%) | | |
| Drinking status | | | | |
| Drink | 17 | (53%) | 0 | (0%) |
| Do not drink | 13 | (41%) | 14 | (100%) |
| Unknown | 2 | (6%) | | |
| HPV16 positive | 11 | (35%) | | |
| Tumor site | | | | |
| Oral cavity | 11 | (35%) | | |
| Oropharynx | 14 | (44%) | | |
| Larynx | 6 | (18%) | | |
| Hypopharynx | 1 | (3%) | | |
| TNM stage | | | | |
| I | 1 | (3%) | | |
| II | 3 | (9%) | | |
| III | 3 | (9%) | | |
| IV | 11 | (35%) | | |
| Unknown | 14 | (44%) | | |
| Disease status | | | | |
| No evidence of | 8 | (25%) | | |
| disease | | | | |
| Alive with disease | 5 | (15%) | | |
| Dead of disease | 4 | (13%) | | |
| Dead of unrelated | 3 | (9%) | | |
| causes | | | | |
| Unknown | 12 | (38%) | | |

### Example 9

### ZNF Downregulation is Associated With Promoter Methylation

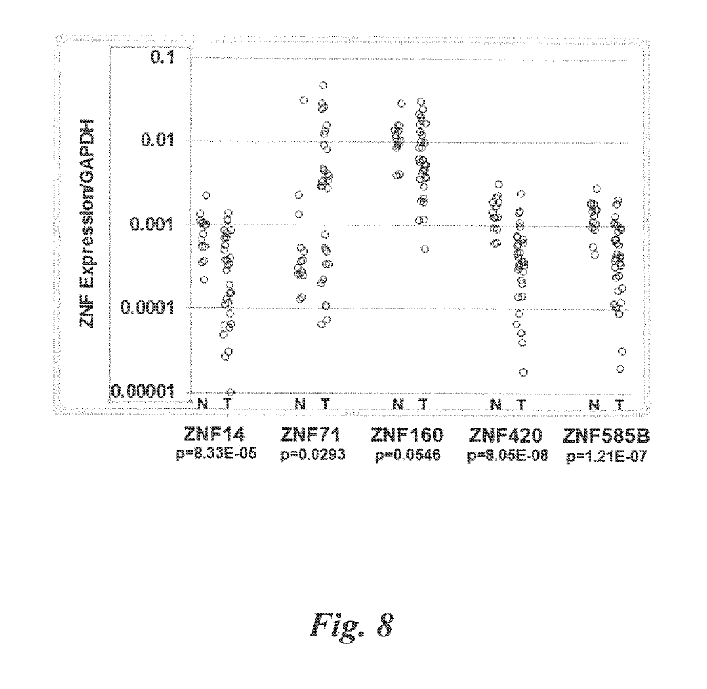

To validate the hypothesis that the expression of individual ZNF protein genes is affected by methylation of their promoter, qRT-PCR analysis was subsequently performed for ZNF14, ZNF71, ZNF160, ZNF420 and ZNF585B expression on the samples from the first validation cohort (FIG. 8). All but ZNF71 demonstrate significant downregulation of ZNF expression in tumor samples as compared to normal tissues in agreement with the increase of their methylation status.

### Example 10

### ZNF DNA Methylation Detection in Primary Tissues and Salivary Rinse in an Expanded Cohort

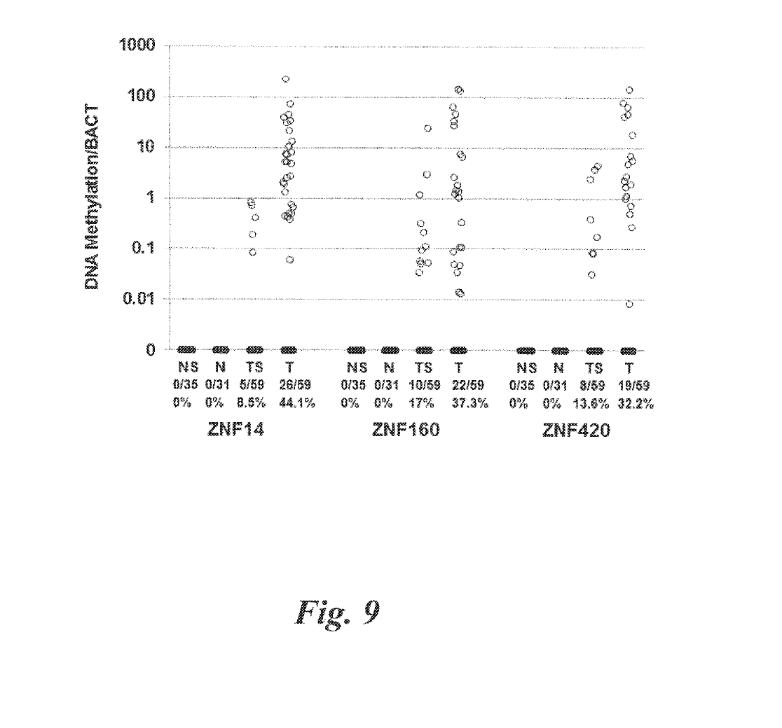

Based on the discovery results, each of ZNF14, ZNF160 and ZNF420 could distinguish HNSCC primary tissues from control normal tissues with sensitivity of at least 25% and specificity of as high as 96% and above. It was hypothesized that DNA methylation signals can be detected with high specificity and specificity in salivary rinse of HNSCC patients. To test this hypothesis, an expanded cohort of salivary rinse and primary tumor cancer tissue from 59 HNSCC patients together with salivary rinse (n=35) and normal uvula tissue samples (n=31) from non-cancerous patients was assembled (Table 4). The DNA methylation signals were determined by QMSP analysis for ZNF14, ZNF160 and ZNF420 promoter methylation with cut-off of no PCR product amplification (FIG. 9).

The distinct methylation pattern in primary tumor samples could be validated, but not validated in non-cancerous tissues for all three ZNF genes with specificity of 100% (95% CI: 88.78%-100%). with the sensitivity of primary HNSCC tissue detection 32.2% for individual genes and 57.63% for the combination of the genes (Table 10). It has been noticed that 17% patients (10 of 59) have all three ZNF promoters methylated. It was also observed that there was a high correlation between the sensitivity and specificity of ZNF promoter methylation in the discovery and both validation cohorts with utilization of three different techniques fbr DNA methylation detection (Table 11).

Comparison of DNA methylation signals in the salivary rinse samples of HNSCC and non-cancerous patients demonstrated that the specificity of HNSCC detection in these body fluids was just as high for all ZNF: 100% (95% Cl: 89,9%-100%), but the sensitivity decreased to 22.03% for the combination of ZNF gene panel (Table 12). The frequency and the detection concordance of DNA methylation for the combined panel of ZNF genes was 35.3% and 92.3%, respectively (Table 13)

Statistical analysis revealed significant correlation (p-value=0.0139) of overall ZNF methylation detected in the primary cancer tissues with oral cavity SCC as opposed to the other tumor sites (Table 26). Individual ZNF's also revealed association of ZNF420 methylation with patient age. Detection of ZNF methylation in HNSCC patient salivary rinse samples also demonstrated correlation with oral cavity SCC as well as with patient smoking history (Table 27). The overall p-values of the association of ZNF methylation status with the clinical characteristics is low due to the modest number of patients with prominent ZNF methylation signals (34 patients with at least one ZNF DNA methylation detected in primary tissues and 13 patients with at least one ZNF DNA methylation detected in salivary rinse).

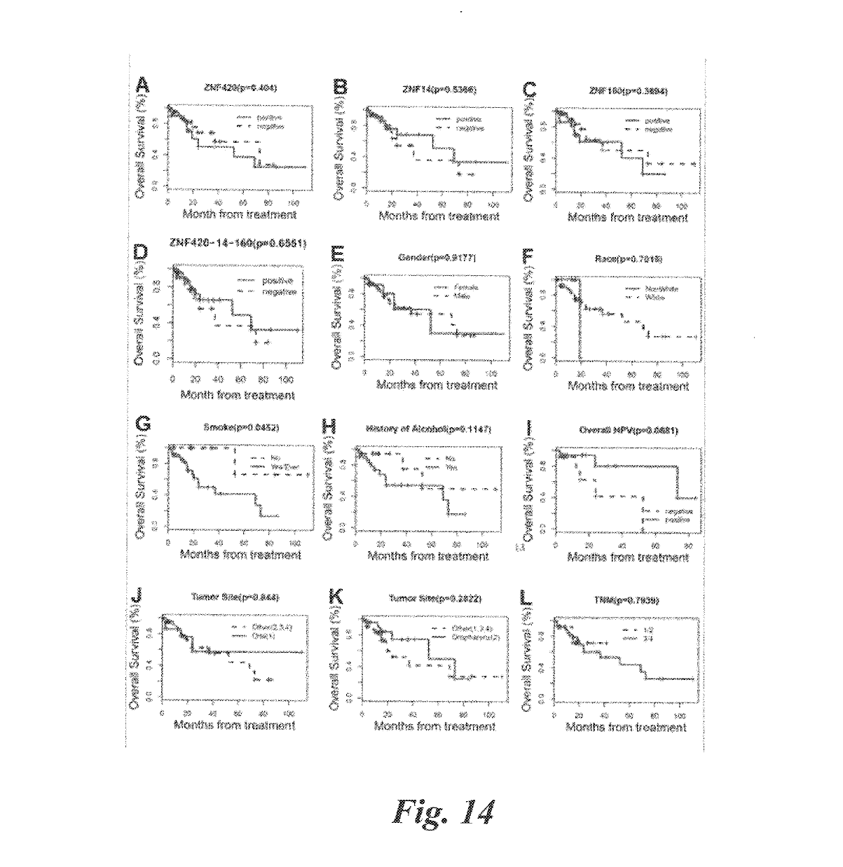

Univariate analysis (Table 24; Cox proportion hazard models were fitted within the second validation HNSCC cohort) and Kaplan-Meier Curve analysis (FIG. 14) did not reveal strong correlation of ZNF methylation with overall HNSCC patient survival. The only statistically significant correlation of HNSCC survival was found with the smoking status of the patients, just as it is expected for the overall HNSCC population (Argiris et al., 2008; Marur and Forastiere, 2008).

TABLE 4
Clinical characteristics of recruited HNSCC patients from the second validation cohort

| | HNSCC | | Normal tissue | | Normal saliva | |
|---|---|---|---|---|---|---|
| | (n = 59) | | (n = 31) | | (n = 35) | |
| | n (%) | | n (%) | | n (%) | |
| Median age (range) | 59 ± 12 | (35-87) | 32 ± 11 | (18-57) | 58 ± 12 | (32-77) |
| Male | 47 | (80%) | 16 | (52%) | 12 | (34%) |
| Female | 12 | (20%) | 15 | (48%) | 23 | (66%) |
| Race | | | | | | |
| Caucasian | 52 | (88%) | 16 | (52%) | 25 | (71%) |
| African American | 5 | (9%) | 12 | (38%) | 7 | (20%) |
| Others | 2 | (3%) | 3 | (10%) | 3 | (9%) |
| Smoking status | | | | | | |
| Packs per year | 43.1 ± 29.9 | (5-110) | 73.9 ± 109.5 | (3-274) | 280 ± 167 | (183-730) |
| (range) | | | | | | |
| Smokers | 47 | (80%) | 9 | (29%) | 18 | (51%) |
| Non-smokers | 12 | (20%) | 22 | (71%) | 17 | (49%) |
| Drinking status | | | | | | |
| Drink | 34 | (58%) | 4 | (13%) | 24 | (69%) |
| Do not drink | 19 | (32%) | 27 | (87%) | 11 | (31%) |
| Unknown | 6 | (10%) | | | | |
| HPV16 positive | 18 | (31%) | | | | |
| Tumor site | | | | | | |
| Oral cavity | 15 | (26%) | | | | |
| Oropharynx | 25 | (42%) | | | | |
| Larynx | 17 | (29%) | | | | |
| Hypopharynx | 2 | (3%) | | | | |
| TNM stage | | | | | | |
| I | 5 | (9%) | | | | |
| II | 10 | (17%) | | | | |
| III | 9 | (15%) | | | | |
| IV | 32 | (54%) | | | | |
| Unknown | 3 | (5%) | | | | |
| Disease status | | | | | | |
| No evidence of | 34 | (58%) | | | | |
| disease | | | | | | |
| Alive with disease | 7 | (12%) | | | | |
| Dead of disease | 7 | (12%) | | | | |
| Dead of unrelated | 11 | (18%) | | | | |
| causes | | | | | | |

TABLE 10
Promoter DNA hypermethylation detection in primary tissues from
HNSCC and non-cancerous patients of the second validation cohort

| | HNSCC | Control | | |
|---|---|---|---|---|
| | (n = 59) | (n = 31) | Sensitivity | Specificity |
| | n | n | % (95% CI) | % (95% CI) |
| ZNF14 | 26 | 0 | 44.07 (31.16- | 100 (88.78- |
| | | | 57.60) | 100) |
| ZNF160 | 23 | 0 | 38.98 (26.55- | 100 (88.78- |
| | | | 52.56) | 100) |
| ZNF420 | 19 | 0 | 32.20 (20.62- | 100 (88.78- |
| | | | 45.64) | 100) |
| Any | 34 | 0 | 57.63 (44.07- | 100 (88.78- |
| ZNF | | | 70.39) | 100) |

TABLE 11
Correlation of the promoter DNA hypermethylation detection
in primary tissues from HNSCC and non-cancerous patients of
three cohorts using three different detection techniques

| | | | 1st Validation | |
|---|---|---|---|---|
| | | Discovery | Cohort | 2nd Validation |
| | | Cohort | Bisulfite | Cohort |
| | | COPA Analysis | Sequencing | QMSP |
| | | Detection in | Detection in | Detection in |
| | | Tu, n = 44 | Tu, n = 32 | Tu, n = 59 |
| | | n (%) | n (%) | n (%) |
| ZNF14 | Sensitivity | 18 (41%) | 14 (43.8%) | 26 (44.1%) |
| | Specificity | 100% | 100% | 100% |
| ZNF160 | Sensitivity | 11 (25%) | 15 (46.88%) | 22 (37.3%) |
| | Specificity | 100% | 86% | 100% |
| ZNF420 | Sensitivity | 15 (34%) | 8 (25%) | 19 (32.2%) |
| | Specificity | 100% | 100% | 100% |

TABLE 12
Promoter DNA hypermethylation detection in salivary rinse of HNSCC
and non-cancerous patients from the second validation cohort

| | HNSCC | Control | | |
|---|---|---|---|---|
| | (n = 59) | (n = 35) | Sensitivity | Specificity |
| | n | n | % (95% CI) | % (95% CI) |
| ZNF14 | 5 | 0 | 8.47 (2.81-18.68) | 100 (89.9-100) |
| ZNF160 | 10 | 0 | 16.95 (8.44-28.97) | 100 (89.9-100) |
| ZNF420 | 8 | 0 | 13.56 (6.04-24.98) | 100 (89.9-100) |
| Any ZNF | 13 | 0 | 22.03 (12.3-34.73) | 100 (89.9-100) |

TABLE 13
Frequency and concordance of DNA methylation signal in plasma with
signal in primary tissues from the second validation cohort

| | Saliva | Tissue | | |
|---|---|---|---|---|
| | (n = 59) | (n = 59) | Frequency | Concordance |
| | n | N | % | % |
| ZNF14 | 5 | 26 | 19.2% | 80% |
| ZNF160 | 10 | 22 | 45.5% | 70% |
| ZNF420 | 8 | 19 | 42.1% | 100% |
| Any ZNF | 13 | 34 | 35.3% | 92.3% |

TABLE 24
Univariate overall survival results. Cox proportion hazard models
were fitted within the second validation HNSCC cohort.

| | Hazard | | |
|---|---|---|---|
| | Ratio | 95% CI | p-Value |
| Methylation marker | | | |
| ZNF420 | 1.49 | (0.58, 3.82) | 0.404 |
| ZNF14 | 0.74 | (0.29, 1.91) | 0.5366 |
| ZNF160 | 1.53 | (0.6, 3.89) | 0.3694 |
| any ZNF | 0.81 | (0.32, 2.06) | 0.6551 |
| Other risk factors | | | |
| Age | 1.04 | (1, 1.08) | 0.0622 |
| Gender | 1.06 | (0.35, 3.23) | 0.9177 |
| Caucasian vs non-Caucasian | 1.5 | (0.19, 11.75) | 0.702 |
| Smoking vs never smoking | 7.95 | (1.05, 60.47) | 0.0452 |
| Drinking vs never drinking | 2.78 | (0.78, 9.9) | 0.1147 |
| HPV | 0.22 | (0.04, 1.12) | 0.0681 |
| Tumor site | | | |
| Oral cavity vs other | 0.89 | (0.29, 2.73) | 0.844 |
| Oropharynx vs other | 0.58 | (0.22, 1.56) | 0.2822 |
| TNM stage | | | |
| III/IV vs I/II | 1.19 | (0.33, 4.25) | 0.7939 |

TABLE 26
Association of combinations of marker presence with patient characteristics
(in tissue) in the second validation HNSCC cohort

| | HNSCC (n = 59) | | | | | |
|---|---|---|---|---|---|---|
| | ZNF14 | | | ZNF160 | | |
| | Methylated | Unmethylated | p- | Methylated | Unmethylated | p- |
| | (n = 26) | (n = 33) | Value | (n = 23) | (n = 36) | Value |
| | n (%) | n (%) | n (%) | n (%) | n (%) | n (%) |
| Median age | 60.12 ± 12.692 | 58.45 ± 10.86 | 0.8845 | 60.48 ± 11.79 | 58.36 ± 11.79 | 0.5593 |
| (range) | (35, 87) | (37, 79) | | (35, 87) | (37, 84) | |
| Male | 23 (88.46) | 24 (72.73) | 0.1963 | 20 (86.96) | 27 (75) | 0.3341 |
| Female | 3 (11.54) | 9 (27.27) | | 3 (13.04) | 9 (25) | |
| Race | | | | | | |
| Caucasian | 24 (92.31) | 28 (84.85) | 0.449 | 20 (86.96) | 32 (88.89) | 1 |
| Non-Caucasian | 2 (7.69) | 5 (15.15) | | 3 (13.04) | 4 (11.11) | |
| Smoking status | | | | | | |
| Smokers | 19 (73.08) | 26 (78.79) | 0.3956 | 16 (69.57) | 29 (80.56) | 0.2311 |
| Former Smokers | 0 (0) | 2 (6.06) | | 0 (0) | 2 (5.56) | |
| Non-smokers | 7 (26.92) | 5 (15.15) | | 7 (30.43) | 5 (13.89) | |
| Drinking status | | | | | | |
| Drink | 18 (69.23) | 16 (48.48) | 0.1603 | 15 (65.22) | 19 (52.78) | 0.3396 |
| Do not drink | 6 (23.08) | 13 (39.39) | | 6 (26.09) | 13 (36.11) | |
| HPV Positive | 7 (26.92) | 11 (33.33) | 0.2852 | 6 (26.09) | 12 (33.33) | 0.2936 |
| Tumor site | | | | | | |
| Oral cavity | 11 (42.31) | 4 (12.12) | 0.0146 | 8 (34.78) | 7 (19.44) | 0.2281 |
| Non oral cavity | 15 (57.69) | 29 (87.88) | | 15 (65.22) | 29 (80.56) | |
| Oropharynx | 11 (42.31) | 14 (42.42) | 1 | 11 (47.83) | 14 (38.89) | 0.5925 |
| Non oropharynx | 15 (57.69) | 19 (57.58) | | 12 (52.17) | 22 (61.11) | |
| TNM stage | | | | | | |
| III/IV | 16 (61.54) | 25 (75.76) | 0.227 | 16 (69.57) | 25 (69.44) | 0.7605 |
| I/II | 9 (34.62) | 6 (18.18) | | 7 (30.43) | 8 (22.22) | |
| | HNSCC (n = 59) | | | | | |
| | ZNF420 | | | any ZNF | | |
| | Methylated | Unmethylated | p- | Methylated | Unmethylated | p- |
| | (n = 19) | (n = 40) | Value | (n = 34) | (n = 25) | Value |
| | n (%) | n (%) | n (%) | n (%) | n (%) | n (%) |
| Median age | 63.74 ± 11.59 | 57.02 ± 11.31 | 0.039 | 59.94 ± 12.17 | 58.16 ± 12.17 | 0.7586 |
| (range) | (41, 87) | (35, 84) | | | | |
| Male | 15 (78.95) | 32 (80) | 1 | 29 (85.29) | 18 (72) | 0.3268 |
| Female | 4 (21.05) | 8 (20) | | 7 (28) | | |
| Race | | | | | | |
| Caucasian | 15 (78.95) | 37 (92.5) | 0.1968 | 29 (85.29) | 23 (92) | 0.6873 |
| Non-Caucasian | 4 (21.05) | 3 (7.5) | | 7 (28) | | |
| Smoking status | | | | | | |
| Smokers | 15 (78.95) | 30 (75) | 1 | 25 (73.53) | 20 (80) | 0.1395 |
| Former Smokers | 0 (0) | 2 (5) | | 0 (0) | 2 (8) | |
| Non-smokers | 4 (21.05) | 8 (20) | | 9 (26.47) | 3 (12) | |
| Drinking status | | | | | | |
| Drink | 13 (68.42) | 21 (52.5) | 0.2352 | 22 (64.71) | 12 (48) | 0.5589 |
| Do not drink | 4 (21.05) | 15 (37.5) | | 10 (29.41) | 9 (36) | |
| HPV Positive | 3 (15.79) | 15 (37.5) | 0.1143 | 9 (26.47) | 9 (36) | 0.4622 |
| Tumor site | | | | | | |
| Oral cavity | 8 (42.11) | 7 (17.5) | 0.058 | 13 (38.24) | 2 (8) | 0.0139 |
| Non oral cavity | 11 (57.89) | 33 (82.5) | | 21 (61.76) | 23 (92) | |
| Oropharynx | 7 (36.84) | 18 (45) | 0.5868 | 14 (41.18) | 11 (44) | 1 |
| Non oropharynx | 12 (63.16) | 22 (55) | | 20 (58.85) | 14 (56) | |
| TNM stage | | | | | | |
| III/IV | 14 (73.68) | 27 (67.5) | 1 | 21 (61.76) | 20 (80) | 0.0694 |
| I/II | 5 (26.32) | 10 (25) | | 12 (35.29) | 3 (12) | |

TABLE 27
Association of marker presence with patient characteristics
(in saliva) in the second validation HNSCC cohort.

| | HNSCC (n = 59) | | | | | |
|---|---|---|---|---|---|---|
| | ZNF14 | | | ZNF160 | | |
| | Methylated | Unmethylated | p- | Methylated | Unmethylated | p- |
| | (n = 5) | (n = 54) | Value | (n = 10) | (n = 49) | Value |
| | n (%) | n (%) | n (%) | n (%) | n (%) | n (%) |
| Median age | 51.24 ± 13.15 | 59.81 ± 11.53 | 0.2 | 61.4 ± 11.53 | 58.73 ± 11.1 | 0.4914 |
| (range) | (41, 73) | (35, 87) | | (35, 87) | (35, 84) | |
| Male | 5 (100) | 42 (77.78) | 0.5725 | 7 (70) | 40 (81.63) | 0.4094 |
| Female | 0 | 12 (22.22) | | 3 (30) | 9 (18.37) | |
| Race | | | | | | |
| Caucasian | 5 (100) | 47 (87.04) | 1 | 9 (90) | 43 (87.76) | 1 |
| Non-Caucasian | 0 | 7 (12.96) | | 1 (10) | 6 (12.24) | |
| Smoking status | | | | | | |
| Smokers | 4 (80) | 41 (75.93) | 1 | 5 (50) | 40 (81.63) | 0.0483 |
| Former Smokers | 0 (0) | 2 (3.7) | | 0 (0) | 2 (4.08) | |
| Non-smokers | 1 (20) | 11 (20.37) | | 5 (50) | 7 (14.29) | |
| Drinking status | | | | | | |
| Drink | 4 (80) | 30 (55.56) | 0.6434 | 6 (60) | 28 (57.14) | 1 |
| Do not drink | 1 (20) | 18 (33.33) | | 4 (40) | 15 (30.61) | |
| HPV Positive | 1 (20) | 17 (31.48) | 0.2836 | 3 (30) | 15 (30.61) | 0.6758 |
| Tumor site | | | | | | |
| Oral cavity | 3 (60) | 12 (12.22) | 0.0986 | 5 (50) | 10 (20.41) | 0.1037 |
| Non oral cavity | 2 (40) | 42 (77.78) | | 5 (50) | 39 (79.59) | |
| Oropharynx | 2 (40) | 23 (42.59) | 1 | 5 (50) | 20 (40.82) | 0.7292 |
| Non oropharynx | 3 (60) | 31 (57.41) | | 5 (50) | 29 (59.18) | |
| TNM stage | | | | | | |
| III/IV | 4 (80) | 37 (68.52) | 1 | 6 (60) | 35 (71.43) | 0.431 |
| I/II | 1 (20) | 14 (25.93) | | 4 (40) | 11 (22.45) | |

| | HNSCC (n = 59) | | | | | |
|---|---|---|---|---|---|---|
| | ZNF420 | | | any ZNF | | |
| | Methylated | Unmethylated | p- | Methylated | Unmethylated | p- |
| | (n = 8) | (n = 51) | Value | (n = 13) | (n = 46) | Value |
| | n (%) | n (%) | n (%) | n (%) | n (%) | n (%) |
| Median age | 65 ± 14.82 | 58.27 ± 11.08 | 0.1494 | 60 ± 14.79 | 58.96 (10.9) | 0.7555 |
| (range) | (41, 87) | (35, 84) | | | | |
| Male | 6 (75) | 41 (80.39) | 0.6597 | 10 (76.92) | 37 (80.43) | 0.716 |
| Female | 2 (25) | 10 (19.61) | | 9 (19.57) | | |
| Race | | | | | | |
| Caucasian | 7 (87.5) | 45 (88.24) | 1 | 12 (92.31) | 40 (86.96) | 1 |
| Non-Caucasian | 1 (12.5) | 6 (11.76) | | 1 (7.69) | 6 (13.04) | |
| Smoking status | | | | | | |
| Smokers | 6 (75) | 39 (76.47) | 0.7544 | 7 (53.85) | 38 (82.61) | 0.0358 |
| Former Smokers | 0 (0) | 2 (3.92) | | 0 (0) | 2 (4.35) | |
| Non-smokers | 2 (25) | 10 (19.61) | | 6 (46.15) | 6 (13.04) | |
| Drinking status | | | | | | |
| Drink | 5 (62.5) | 29 (56.86) | 1 | 7 (53.85) | 27 (58.7) | 0.7362 |
| Do not drink | 2 (25) | 17 (33.33) | | 5 (38.46) | 14 (30.43) | |
| HPV Positive | 1 (12.5) | 17 (33.33) | 0.1337 | 3 (23.08) | 15 (32.61) | 0.413 |
| Tumor site | | | | | | |
| Oral cavity | 5 (62.5) | 10 (19.61) | 0.0202 | 8 (61.54) | 7 (15.22) | 0.0019 |
| Non oral cavity | 3 (37.5) | 41 (80.39) | | 5 (38.46) | 39 (84.78) | |
| Oropharynx | 3 (37.5) | 22 (43.14) | 1 | 5 (38.46) | 20 (43.48) | 1 |
| Non oropharynx | 5 (62.5) | 29 (56.86) | | 8 (61.54) | 26 (56.52) | |
| TNM stage | | | | | | |
| III/IV | 6 (75) | 35 (68.63) | 1 | 7 (53.85) | 34 (73.91) | 0.087 |
| I/II | 2 (25) | 13 (25.49) | | 6 (46.15) | 9 (19.57) | |

### Example 11

### Evaluation of the ZNF Protein Function for HNSCC Cells

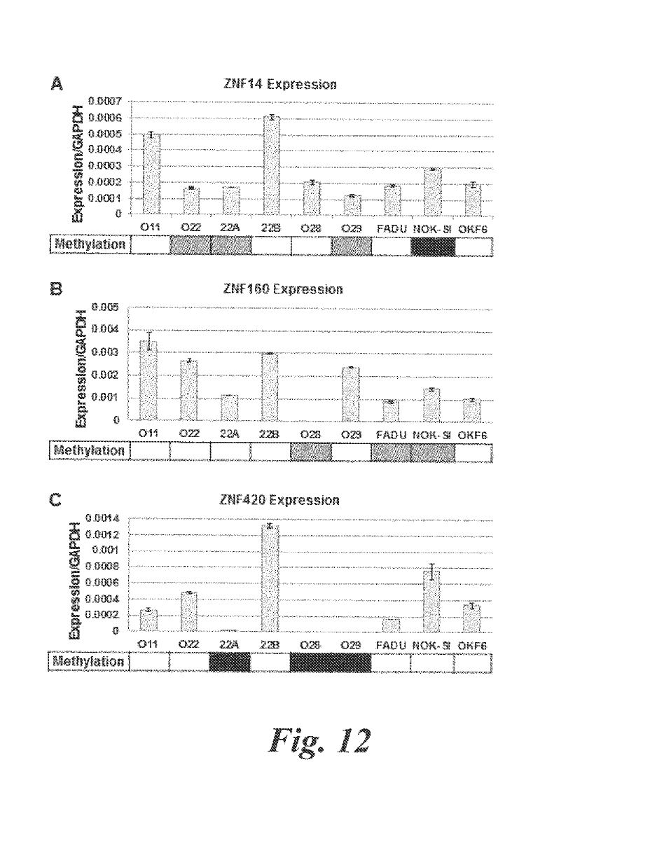

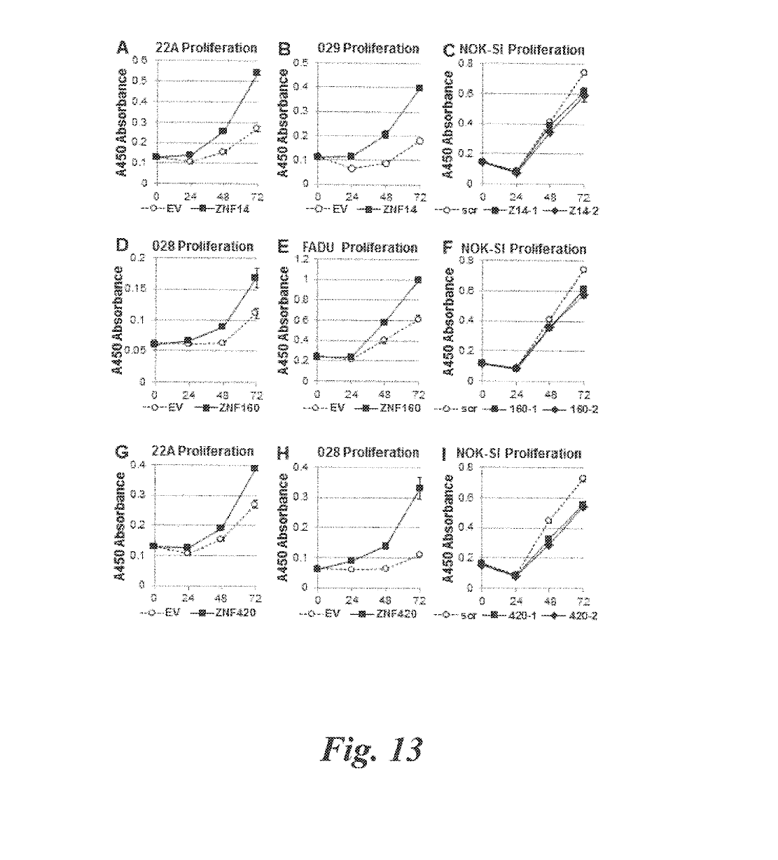

There is not much information available regarding the functions of ZNF14, ZNF160 and ZNF420. The most recent data suggest that ZNF420, also known as Apak, is a suppressor of p53-mediated apoptosis and that it plays a cell growth promoting effect on human osteosarcoma cells (Tian et al., 2009; Wang et al., 2010b). On the other hand, ZNF420 is shown to be inhibited and inactivated by DNA damage and oncogenic stress (Wang et al., 2010b). ZNF160 was shown to play a role as a negative epigenetic regulator for TLR4 (toll-like receptor 4) gene expression (Takahashi et al., 2009). TLR4 is overexpressed in several types of tumors (Takahashi et al., 2009; Wang et al., 2010a), suggesting tumor-suppressing function of ZNF160, ZNF14 was shown to stimulate expression and activation of the putative tumor suppressor ERβ (Kouzu-Fujita et al., 2009). These data suggest that while ZNF14 and ZNF160 may be tumor-suppressing genes, ZNF420 may be a prospective oncogene. To resolve these conflicting reports data and to elucidate the role of these identified ZNF proteins in HNSCC progression, model head and neck cancer and normal cell lines were tested using cell proliferation assays. ZNF promoter methylation and gene expression in the cell lines confirmed the methylation dependent regulation of ZNF genes expression (FIG. 12). The expression of ZNF proteins in the model cell lines was both induced and knocked-down (FIG. 13). Results of the cell proliferation assay suggest that these described ZNF may have oncogenic properties. Due to the artificial experimental condition and reported difference of cell lines from the primary tissues, the exact function of chosen ZNF in head and neck cancer could not be concluded. Such data suggest that changes in ZNF expression were caused by cancer-related changes of the methylation status of those genes.

### Example 12

### Further Discussion

The recent development of high-throughput array platforms has greatly enhanced the molecular characterization of HNSCC (Akavia et al., 2010; Chung et al., 2004; Smith et al., 2009; Smith et al., 2007). However, the discovery of cancer-causing aberrations based on single platform analysis is limited by individual array bias. A novel biostatistical method was developed to uncover HNSCC drivers by the integration of data from gene expression and DNA methylation high-throughput arrays for 44 HNSCC and 25 normal control tissue samples. The analysis included manipulation of the traditional Cancer Outlier Profile Analysis (COPA) to a two-sided COPA that can be applied to both oncogenes and tumor suppressor genes for expression and methylation signals. Two-sided COPA allowed the identification of 118 prospective cancer-associated genes, the majority of which have never been reported for HNSCC. Thirty six of them demonstrated strong expression-methylation anticorrelation. The methylation status was validated for twenty-six of the thirty-six (72%) genes. Out of the. twenty six validated genes, twenty were described for the validation on the independent cohort, where fourteen of them (70%) demonstrated strong differential methylation in tumors, as compared to normal tissues.

Of those fourteen genes, the focus has been on three newly discovered tumor-suppressing zinc finger proteins: ZNF14, ZNF160 and ZNF420. These genes demonstrated significant downregulation in tumor samples that strongly correlated with DNA methylation. Quantitative methylation-specific PCR (QMSP)-based assays determined that their DNA methylation signals could he detected in primary HNSCC tissues and matched salivary rinse samples. DNA methylation of at least one ZNF was detected in primary cancer tissues with sensitivity of 57.63% (95% CI: 44.07%-70.39%) and specificity of 100% (95% CI; 88.78%-100%). In addition, detection of DNA methylation in salivary rinse of at least one ZNF had 22.03% sensitivity (95% CI: 12.3% to 34.73%) and 100% specificity (95% CI: 89.9%-100%). Detection concordance and frequency of DNA methylation is salivary' rinse was 92.3% and 35.3%, respectively, as compared to the primary cancer tissues.

Functional analysis suggests that ZNF hypermethylation in tumors is a cancer-passenger event that leads to downregulation of ZNF expression, thereby supporting the recent discoveries regarding the hypermethylation of ZNF cluster in chromosome 19 in Oropharynx SCC (Lieras et at, 2011).

In this study, a novel integrative screening strategy was used to specifically look fir aberrantly expressed genes in human HNSCC whose transcription changes are driven by promoter methylation. For this reason, multiple modern high-throughput techniques were employed to perform genome-wide screening of gene expression and DNA methylation on a relatively large cohort of 44 primary HNSCC and 25 normal tissues. To increase the specificity of the screening data, highly rigorous standards were used and the analysis was limited to 22 thousand core probes for the expression data (out of 1.4 million array probes) and to 1.2 thousand core probes for the methylation data (out of 28 thousand array probes). Analysis of those probes demonstrated that certain genes were aberrantly expressed and other genes had significant changes in their methylation status in tumor samples as compared to the normal tissues. To integrate data from both platforms, several biostatistical approaches were employed; COPA analysis was used to define differentially methylated and differentially expressed genes. COPA scores were calculated for both upper-tail and lower-tail genes from both arrays: this allowed definition of outliers that are overexpressed, downregulated, hypermethylated and hypomethylated. The analysis was limited to the top COPA-scoring genes from each array, giving 81 and 37 genes from the expression and the methylation arrays. It should be noted that by using less rigorous standards a bigger number of candidates could have been obtained, but analysis of more genes was not within the scope of this project. Out of the total 118 genes, 36 with the strongest expression-methylation correlation have been depicted.

All chosen candidate genes were thoroughly validated for their methylation status using bisulfite sequencing to estimate the absolute level of CpG island methylation. 72% genes were successfully validated to have differential methylation status in the tumor samples from the original discovery cohort. The majority of the genes were also validated in the additional validation cohort, confirming that at least 70% of these genes have differential methylation in tumor samples, strongly supporting the data from the discovery cohort.

It was intended to elucidate the global pathway-based changes in expression and methylation of candidate genes, and that is why attention was focused on seven ZNF protein genes in the top-scoring 20 candidate genes. Out of seven ZNF genes, four demonstrated strong correlation between significantly increased methylation and decrease expression in tumors in the independent cohort of samples (FIG. 8). These four genes, namely ZNF14, ZNF160, ZNF420, and ZNF585B are transcription regulators with a conserved Kruppel associated box domain (KRAB-A) that is a transcription repression module (Coleman, 1992; Vissing et al., 1995; Vogel et al., 2006; Witzgall et al., 1994). Proteins containing a KRAB-A domain play important roles in cell differentiation and organ development, and in regulating viral replication and transcription. Also, all four of them are located on Chromosome 19, which was shown to be hypermethylated in oropharyngeal cancer cases (Halford et al., 1995; Lleras et al., 2011; Tian et al., 2009). A parallel study was conducted using the same 44 primary HNSCC and 25 normal mucosal samples to determine copy number on the Affymetrix Genome-wide SNP 6.0 Array containing 950,000 copy number probes. The copy number for all four genes was checked and no significant difference in copy number was found for all four genes. It is more likely that this region is epigenetically silenced during HNSCC progression and the mechanism for epigenetic regulation of these ZNF genes is yet unknown. Out of the other three genes, ZNF71 showed positive correlation between expression and methylation during validation (Compare FIG. 7 and FIG. 8 for ZNF71), proposing that ZNF71 expression is regulated by means other than promoter CpG island methylation. The differential methylation status of two more ZNF was not validated.

Out of the twenty genes chosen for validation in a separate cohort by bisulfite sequencing, seven genes showed more than 35% methylation difference between normal mucosal samples and primary HNSCC tissues namely: BANK1, INA, MAP4K1, HHEX, DTX1, BIN2 and HAAO (FIG. 7). Two more genes, CLGN and FUZ showed less than 35% difference in methylation, which may improve if by using a larger sample size cohort was employed.

Out of the fourteen validated genes mentioned above (FIG. 7), the experiments focused on the application of three ZNF genes for HNSCC gene or regulatory region development. With high specificity and sensitivity, ZNF methylation signals were detected in primary tumor samples and salivary rinse samples of HNSCC patients (Tables 10 and 12), suggesting the potency of those genes in the development of the clinically-applicable tumor-detection techniques. The rate of aberrant DNA methylation of these ZNF was comparable in the discovery and both validation cohorts, despite using three different techniques for DNA methylation detection, methylation array, bisulfite sequencing and QMSP, on three independent cohorts (Table 11). Detection of ZNF DNA methylation in salivary rinse also demonstrated high frequency and strong concordance with DNA detection in primary tissues (Table 13).

Statistical analysis did not reveal strong association of ZNF methylation with clinical characteristics, known risk factors or overall HNSCC patient survival. Overall ZNF methylation detection correlated with tumor site and patient smoking status. This can be explained by the small number of patients with detected ZNF methylation in both primary tumor samples and cancer patient salivary rinse samples. This correlation can be improved by employment of the larger group of patients or combination of ZNF detection with previously discovered DNA methylation markers of HNSCC (Carvalho et al., 2006; Carvalho et al., 2008; Demokan et 2010; Pattani et al., 2010)

Hypermethylation and downregulation of the described ZNF in tumors as well as presence of KRAB domain in their structure strongly supports their proposed tumor-suppressor function. ZNF14 was shown to stimulate the expression and activation of the putative tumor suppressor ERbeta (Bossard et al., 2012; Kouzu-Fujita et al., 2009), supporting its proposed role as a tumor-suppressor. ZNF160 was shown to negatively regulate the expression of TLR4, which is overexpressed in several types of cancer (Takahashi et al 2009; Wang et at, 2010a); this report also supports its function as a tumor-suppressor function. On the other hand, ZNF420 was shown to suppress p53 mediated apoptosis, induce osteosarcoma cell proliferation, and it was shown to he inactivated and suppressed under oncogenic stress conditions (Tian et al 2009; Wang et al, 2010b). Such data suggest a role opposite to that of ZNF14 and ZNF160. The functional studies performed on head and neck cancer and normal cell lines demonstrated that all analyzed ZNF induce cell proliferation. The results suggest that the DNA methylation driven by carcinogenesis is the main regulator of ZNF gene expression, while ectopic overexpression or down-regulation of these genes in cultured cell lines activated parallel pathways that affect gene proliferation independent of the methylation and expression status of ZNF genes.

Overall the data suggest that utilization of ZNF DNA methylation especially in the combination with previously discovered DNA methylation biomarkers of HNSCC in the clinical practice for non-invasive detection of HNSCC can strongly improve early detection of HNSCC especially in the risk group patients (smokers with oral cavity SCC). This hypothesis is partially supported by low p-value (0.087) of the detection of at least one ZNF in the salivary rinse of HNSCC patients with earlier I and II cancer stage and require further investigation.

### Example 13

### Patient Characteristics in HNSCC Cohort, Normal Tissue Cohort, and Normal Saliva Cohort

Table 14 shows the summary statistics of patient characteristics used herein. P-values for testing the differences between groups were based on the fisher exact test, or the wilcoxen test upon property (P-value<0.0001 denote as p-value=0). Expression levels were zero in the entire normal tissue and normal saliva cohort.

The summary statistics of marker characteristics are shown in Table 15. The P-value for testing the differences between groups are based on the fisher exact test (P-value<0,0001 is denoted as p-value=0).

TABLE 14
Summary statistics of patient characteristics.

| | #(%) | | | | | |
|---|---|---|---|---|---|---|
| | | | | p-value | | |
| | Cohorts | | | | | p(normal |
| | | normal | normal | p(HNSCC | p(HNSCC | tissue |
| | HNSCC | tissue | saliva | vs normal | vs normal | vs normal |
| | (n = 59) | (n = 31) | (n = 35) | tissue) | saliva) | saliva) |
| Age | | | | | | |
| mean(sd) | 55.19 (11.73) | 31.84 (10.81) | 58.29 (12.12) | 0 | 0.8418 | 0 |
| median(range) | 61 (35, 87) | 30 (18, 57) | 58 (32, 77) | | | |
| Gender | | | | | | |
| M | 47 (79.66) | 16 (51.61) | 12 (34.29) | 0.008 | 0 | 0.2132 |
| F | 12 (20.34) | 15 (48.39) | 23 (65.71) | | | |
| Race | | | | | | |
| 1 (White) | 52 (88.14) | 16 (51.61) | 25 (71.43) | 5e−04 | 0.1665 | 0.2938 |
| 2 (Black) | 5 (8.47) | 12 (38.71) | 7 (20) | | | |
| 3 (Asian) | 1 (1.69) | 2 (6.45) | 1 (2.86) | | | |
| 4 (Other) | 1 (1.69) | 1 (3.23) | 2 (5.71) | | | |
| Race | | | | | | |
| White | 52 (88.14) | 16 (51.61) | 25 (71.43) | 2e−04 | 0.0542 | 0.1293 |
| NonWhite | 7 (11.86) | 15 (48.39) | 10 (28.57) | | | |
| Smoke | | | | | | |
| 0(Never) | 12 (20.34) | 22 (70.97) | 17 (48.57) | 0 | 0 | 0.0045 |
| 1(Yes) | 45 (76.27) | 7 (22.58) | 4 (11.43) | | | |
| 2(Former) | 2 (3.39) | 2 (6.45) | 14 (40) | | | |
| Alcohol | | | | | | |
| 0 (No) | 19 (32.2) | 27 (87.1) | 11 (31.43) | 0 | 0.8187 | 0 |
| 1 (Yes) | 34 (57.63) | 4 (12.9) | 24 (68.57) | | | |
| Overall HPV | | | | | | |
| positive(1) | 18 (30.51) | | | 0.1575 | | |
| negative(0) | 13 (22.03) | | | | | |
| Tumor Site | | | | | | |
| 1(Oral cavity) | 15 (25.42) | | | | | |
| 2(oropharynx) | 25 (42.37) | | | | | |
| 3(larynx) | 17 (28.81) | | | | | |
| 4(hypopharynx) | 2 (3.39) | | | | | |
| 5(sinonasal) | 0 | | | | | |
| Tumor Site | | | | | | |
| 1(Oral cavity) | 15 (25.42) | | | 0 | | |
| Other(2, 3, 4) | 44 (74.58) | | | | | |
| Tumor Site | | | | | | |
| 2(Oropharynx) | 25 (42.37) | | | 0.0251 | | |
| Other(1, 3, 4) | 34 (57.63) | | | | | |
| T stage | | | | | | |
| T0 | 1 (1.69) | | | | | |
| T1 | 11 (18.64) | | | | | |
| T2 | 18 (30.51) | | | | | |
| T3 | 13 (22.03) | | | | | |
| T4 | 9 (15.25) | | | | | |
| T4a | 5 (8.47) | | | | | |
| Tx | 2 (3.39) | | | | | |
| T stage | | | | | | |
| T1/T2 | 29 (49.15) | | | 0.6961 | | |
| T3/T4 | 27 (45.76) | | | | | |
| N stage | | | | | | |
| N0 | 16 (27.12) | | | | | |
| N1 | 7 (11.86) | | | | | |
| N2 | 25 (42.37) | | | | | |
| N3 | 1 (1.69) | | | | | |
| Nx | 10 (16.95) | | | | | |
| N stage | | | | | | |
| N1/N2/N3 | 33 (55.93) | | | 0 | | |
| N0 | 16 (27.12) | | | | | |
| N stage | | | | | | |
| N2/N3 | 26 (44.07) | | | 0.5046 | | |
| N1/N0 | 23 (38.98) | | | | | |
| M stage | | | | | | |
| Mx | 58 (98.31) | | | | | |
| Anatomic.TNM.stage | | | | | | |
| 1 | 5 (8.47) | | | | | |
| 2 | 10 (16.95) | | | | | |
| 3 | 9 (15.25) | | | | | |
| 4 | 32 (54.24) | | | | | |
| ND | 3 (5.08) | | | | | |
| Anatomic.TNM.stage | | | | | | |
| 3/4 | 41 (69.49) | | | | | |
| 1/2 | 15 (25.42) | | | | | |

P-values for testing the differences between groups were based on fisher exact test, or wilcoxon test upon property.
P-value < 0.0001 denote as p-value = 0.
Note:
Expression levels were zero in the entire normal tissue and normal saliva cohort.

TABLE 15
Summary statistics of marker characteristics.

| | # (%) | | | | | |
|---|---|---|---|---|---|---|
| | | | | p-value | | |
| | Cohorts | | | | | p(normal |
| | | normal | normal | p(HNSCC | p(HNSCC | tissue |
| | HNSCC | tissue | saliva | vs normal | vs normal | vs normal |
| | (n = 59) | (n = 31) | (n = 35) | tissue) | saliva) | saliva) |
| Tissue | | | | | | |
| ZNF420 | | | | | | |
| negative | 40 (67.8) | 31 (100) | — | 2e−04 | — | |
| positive | 19 (32.2) | 0 (0) | — | | — | |
| ZNF14 | | | | | | |
| negative | 33 (55.93) | 31 (100) | — | 0 | — | |
| positive | 26 (44.97) | 0 (0) | — | | — | |
| ZNF160 | | | | | | |
| negative | 36 (61.02) | 31 (100) | — | 0 | — | |
| positive | 23 (38.98) | 0 (0) | — | | — | |
| Combination ¹ | | | | | | |
| ZNF420, ZNF14 | | | | | | |
| negative | 29 (49.15) | 31 (100) | — | 0 | — | |
| positive | 30 (50.85) | 0 (0) | — | | — | |
| ZNF420, ZNF160 | | | | | | |
| negative | 30 (50.85) | 31 (100) | — | 0 | — | |
| positive | 29 (49.15) | 0 (0) | — | | — | |
| ZNF14, ZNF160 | | | | | | |
| negative | 26 (44.07) | 31 (100) | — | 0 | — | |
| positive | 33 (55.93) | 0 (0) | — | | — | |
| ZNF420, ZNF14, ZNF160 | | | | | | |
| negative | 25 (42.37) | 31 (100) | — | 0 | — | |
| positive | 34 (57.63) | 0 (0) | — | | — | |
| Saliva | | | | | | |
| ZNF420 | | | | | | |
| negative | 51 (86.44) | — | 35 (100) | — | 0.0237 | |
| positive | 8 (13.56) | — | 0 (0) | — | | |
| ZNF14 | | | | | | |
| negative | 54 (91.53) | — | 35 (100) | — | 0.1534 | |
| positive | 5 (8.47) | — | 0 (0) | — | | |
| ZNF160 | | | | | | |
| negative | 49 (83.05) | — | 35 (100) | — | 0.0119 | |
| positive | 10 (16.95) | — | 0 (0) | — | | |
| Combination | | | | | | |
| ZNF420, ZNF14 | | | | | | |
| negative | 50 (84.75) | — | 35 (100) | — | 0.024 | |
| positive | 9 (15.25) | — | 0 (0) | — | | |
| ZNF420, ZNF160 | | | | | | |
| negative | 47 (79.66) | — | 35 (100) | — | 0.0031 | |
| positive | 12 (20.34) | — | 0 (0) | — | | |
| ZNF14, ZNF160 | | | | | | |
| negative | 47 (79.66) | — | 35 (100) | — | 0.0031 | |
| positive | 12 (20.34) | — | 0 (0) | — | | |
| ZNF420, ZNF14, ZNF160 | | | | | | |
| negative | 46 (77.97) | — | 35 (100) | — | 0.0016 | |
| positive | 13 (22.03) | — | 0 (0) | — | | |
| Plasma | | | | | | |
| ZNF420 | | | | | | |
| negative | 58 (98.31) | — | — | — | — | — |
| positive | 1 (1.69) | — | — | — | — | — |
| ZNF14 | | | | | | |
| negative | 58 (98.31) | — | — | — | — | — |
| positive | 1 (1.69) | — | — | — | — | — |
| ZNF160 | | | | | | |
| negative | 57 (96.61) | — | — | — | — | — |
| positive | 2 (3.39) | — | — | — | — | — |
| Combination | | | | | | |
| ZNF420, ZNF14 | | | | | | |
| negative | 58 (98.31) | — | — | — | — | — |
| positive | 1 (1.69) | — | — | — | — | — |
| ZNF420, ZNF160 | | | | | | |
| negative | 57 (96.61) | — | — | — | — | — |
| positive | 2 (3.39) | — | — | — | — | — |
| ZNF14, ZNF14160 | | | | | | |
| negative | 57 (96.61) | — | — | — | — | — |
| positive | 2 (3.39) | — | — | — | — | — |
| ZNF420, ZNF14, ZNF160 | | | | | | |
| negative | 57 (96.61) | — | — | — | — | — |
| positive | 2 (3.39) | — | — | — | — | — |

P-value for testing the differences between groups are based on fisher exact test.
P-value < 0.0001 is denoted as p-value = 0.
¹ Combination markers were defined as positive if expression level in any interested combination markers above 0

### Example 14

### Sensitivity and Specificity (HNSCC Tumor vs. Normal Tissue, HNSCC Saliva vs. Normal Saliva, and HNSCC Plasma vs. Normal Plasma)

The sensitivity and specificity of predicting tumor is shown in Table 16. The association of marker presence with patient characteristics in tissue in the HNSCC cohort is shown in Table 17. Further, the association of combinations of marker presence with patient characteristics in tissue in the HNSCC cohort is shown in Table 18. The association of marker presence with patient characteristics (Table 19) and the association of combinations of marker presence (Table 20) in saliva in the HNSCC cohort are also shown. In addition, the association of marker presence with patient characteristics (Table 21) and the association of combinations of marker presence (Table 22) in plasma in the HNSCC cohort are shown.

TABLE 16
Sensitivity and Specificity of Predicting Tumor

| | Tissue samples (n = 90) | | Saliva samples (n = 94) | |
|---|---|---|---|---|
| % (95% C.I.) | sensitivity | specificity | sensitivity | specificity |
| Marker | | | | |
| ZNF420 | 32.2 (20.62, 45.64) | 100 (88.78, 100) | 13.56 (6.04, 24.98) | 100 (90, 100) |
| ZNF14 | 44.07 (31.16, 57.6) | 100 (88.78, 100) | 8.47 (2.81, 18.68) | 100 (90, 100) |
| ZNF160 | 38.98 (26.55, 52.56) | 100 (88.78, 100) | 16.95 (8.44, 28.97) | 100 (90, 100) |
| Combination ² | | | | |
| ZNF420, ZNF14 | 50.85 (37.5, 64.11) | 100 (88.78, 100) | 15.25 (7.22, 26.99) | 100 (90, 100) |
| ZNF420, ZNF160 | 49.15 (35.89, 62.5) | 100 (88.78, 100) | 20.34 (10.98, 32.83) | 100 (90, 100) |
| ZNF14, ZNF160 | 55.93 (42.4, 68.84) | 100 (88.78, 100) | 20.34 (10.98, 32.83) | 100 (90, 100) |
| ZNF420, ZNF14, ZNF160 | 57.63 (44.07, 70.39) | 100 (88.78, 100) | 22.03 (12.29, 34.73) | 100 (90, 100) |

TABLE 17
: Assocation of marker presence with patient characteristics (in tissue) in HNSCC cohort.

| | # (%) | | | | |
|---|---|---|---|---|---|
| | HNSCC tissue(n = 59) | | | | |
| | ZNF420 | | | | |
| | | | p- | ZNF14 | |
| | pos | neg | value | pos | neg |
| Age | | | | | |
| mean(sd) | 63.74 (11.59) | 57.02 (11.31) | 0.039 | 60.12 (12.92) | 58.45 (10.86) |
| median(range) | 64 (41, 87) | 57.5 (35, 84) | | 58 (35, 87) | 61 (37, 79) |
| Gender | | | | | |
| Male | 15 (78.95) | 32 (80) | 1 | 23 (88.46) | 24 (72.73) |
| Female | 4 (21.05) | 8 (20) | | 3 (11.54) | 9 (27.27) |
| Race | | | | | |
| White | 15 (78.95) | 37 (92.5) | 0.1968 | 24 (92.31) | 28 (84.85) |
| Nonwhite | 4 (21.05) | 3 (7.5) | | 2 (7.69) | 5 (15.15) |
| Smoke | | | | | |
| 0 (Never) | 4 (21.05) | 8 (20) | 1 | 7 (26.92) | 5 (15.15) |
| 1(Yes) | 15 (78.95) | 30 (75) | | 19 (73.08) | 26 (78.79) |
| 2(Former) | 0 (0) | 2 (5) | | 0 (0) | 2 (6.06) |
| Alcohol | | | | | |
| 0(No) | 4 (21.05) | 15 (37.5) | 0.2352 | 6 (23.08) | 13 (39.39) |
| 1(Yes) | 13 (68.42) | 21 (52.5) | | 18 (69.23) | 16 (48.48) |
| Overall HPV | | | | | |
| 1(positive) | 3 (15.79) | 15 (37.5) | | 7 (26.92) | 11 (33.33) |
| 0(negative) | 6 (31.58) | 7 (17.5) | 0.1143 | 8 (30.77) | 5 (15.15) |
| Tumor Site | | | | | |
| 1(Oral cavity) | 8 (42.11) | 7 (17.5) | | 11 (42.31) | 4 (12.12) |
| Other(2, 3, 4) | 11 (57.89) | 33 (82.5) | 0.058 | 15 (57.69) | 29 (87.88) |
| Tumor Site | | | | | |
| 2(Oropharynx) | 7 (36.84) | 18 (45) | | 11 (42.31) | 14 (42.42) |
| Other(1, 3, 4) | 12 (63.16) | 22 (55) | 0.5808 | 15 (57.69) | 19 (57.58) |
| T Stage | | | | | |
| T3/T4 | 10 (52.63) | 17 (42.5) | | 10 (38.46) | 17 (51.52) |
| T1/T2 | 9 (47.37) | 20 (50) | 0.7789 | 14 (53.85) | 15 (45.45) |
| N Stage | | | | | |
| N1/N2/N3 | 10 (52.63) | 23 (57.5) | | 12 (46.15) | 21 (63.64) |
| N0 | 6 (31.58) | 10 (25) | 0.7475 | 8 (30.77) | 8 (24.24) |
| N Stage | | | | | |
| N2/N3 | 8 (42.11) | 18 (45) | | 9 (31.62) | 17 (51.52) |
| N0/N1 | 8 (42.11) | 15 (37.5) | 1 | 11 (42.31) | 12 (36.36) |
| Anatomic.TNM.stage | | | | | |
| 3/4 | 14 (73.68) | 27 (67.5) | 1 | 16 (61.54) | 25 (75.76) |
| 1/2 | 5 (26.32) | 10 (25) | | 9 (34.62) | 6 (18.18) |

| | # (%) | | | |
|---|---|---|---|---|
| | HNSCC tissue(n = 59) | | | |
| | ZNF14 | ZNF160 | | |
| | p- | | | p- |
| | value | pos | neg | value |
| Age | | | | |
| mean(sd) | 0.8845 | 60.48 (11.79) | 58.36 (11.79) | 0.5593 |
| median(range) | | 60 (35, 87) | 61 (37, 84) | |
| Gender | | | | |
| Male | 0.1963 | 20 (86.96) | 27 (75) | 0.3341 |
| Female | | 3 (13.04) | 9 (25) | |
| Race | | | | |
| White | 0.449 | 20 (86.96) | 32 (88.89) | 1 |
| Nonwhite | | 3 (13.04) | 4 (11.11) | |
| Smoke | | | | |
| 0 (Never) | 0.3956 | 7 (30.43) | 5 (13.89) | 0.2311 |
| 1(Yes) | | 16 (69.57) | 29 (80.56) | |
| 2(Former) | | 0 (0) | 2 (5.56) | |
| Alcohol | | | | |
| 0(No) | 0.1603 | 6 (26.09) | 13 (36.11) | 0.3906 |
| 1(Yes) | | 15 (65.22) | 19 (52.78) | |
| Overall HPV | | | | |
| 1(positive) | | 6 (26.09) | 12 (33.33) | 0.2936 |
| 0(negative) | 0.2852 | 7 (30.43) | 6 (16.67) | |
| Tumor Site | | | | |
| 1(Oral cavity) | | 8 (34.78) | 7 (19.44) | 0.2281 |
| Other(2, 3, 4) | 0.0146 | 15 (65.22) | 29 (80.56) | |
| Tumor Site | | | | |
| 2(Oropharynx) | | 11 (47.83) | 14 (38.89) | 0.5925 |
| Other(1, 3, 4) | 1 | 12 (52.17) | 22 (61.11) | |
| T Stage | | | | |
| T3/T4 | | 9 (39.13) | 18 (50) | 0.4226 |
| T1/T2 | 0.4303 | 13 (56.52) | 16 (44.44) | |
| N Stage | | | | |
| N1/N2/N3 | | 12 (52.17) | 21 (58.33) | 0.7565 |
| N0 | 0.5362 | 7 (39.43) | 9 (25) | |
| N Stage | | | | |
| N2/N3 | | 9 (39.13) | 17 (47.22) | 0.5688 |
| N0/N1 | 0.3944 | 10 (43.48) | 13 (36.44) | |
| Anatomic.TNM.stage | | | | |
| 3/4 | 0.227 | 16 (69.57) | 25 (69.44) | 0.7605 |
| 1/2 | | 7 (30.43) | 8 (22.22) | |

TABLE 18
Association of combinations of marker presence with patient characteristics (in tissue) in HNSCC cohort.

| # (%) | |
|---|---|
| HNSCC tissue(n = 59) | |
| ZNF420_14 | ZNF420_160 |

| | | | p- | | | p- |
|---|---|---|---|---|---|---|
| | pos | neg | value | pos | neg | value |
| Age | | | | | | |
| mean(sd) | 60.53 (12.4) | 57.79 (11.05) | 0.569 | 60.48 (11.67) | 57.93 (11.86) | 0.395 |
| median(range) | 59.5 (35, 87) | 61 (37, 79) | | 62 (35, 87) | 59.5 (37, 84) | |
| Gender | | | | | | |
| Male | 25 (83.33) | 22 (75.86) | 0.5321 | 24 (82.76) | 23 (76.67) | 6.748 |
| Female | 5 (16.67) | 7 (24.14) | | 5 (17.24) | 7 (23.33) | |
| Race | | | | | | |
| White | 20 (83.33) | 27 (93.1) | 0.4236 | 25 (86.21) | 27 (90) | 0.7065 |
| Nonwhite | 5 (16.67) | 2 (6.9) | | 4 (13.79) | 3 (10) | |
| Smoke | | | | | | |
| 0 (Never) | 8 (26.67) | 4 (13.79) | 0.2172 | 8 (27.59) | 4 (13.33) | 0.1827 |
| 1(Yes) | 22 (73.33) | 23 (79.31) | | 21 (72.41) | 24 (80) | |
| 2(Former) | 0 (0) | 2 (6.9) | | 0 (0) | 2 (6.87) | |
| Alcohol | | | | | | |
| 0(No) | 8 (26.67) | 11 (37.93) | 0.2671 | 8 (27.59) | 11 (36.67) | 0.3983 |
| 1(Yes) | 20 (66.67) | 14 (48.28) | | 19 (65.52) | 15 (50) | |
| Overall HPV | | | | | | |
| 1(positive) | 7 (23.33) | 11 (37.93) | | 8 (27.59) | 10 (33.33) | |
| 0(negative) | 9 (30) | 4 (13.79) | 0.1489 | 8 (27.59) | 5 (16.67) | 0.4725 |
| Tumor Site | | | | | | |
| 1(Oral cavity) | 12 (40) | 3 (40.34) | | 11 (37.93) | 4 (43.33) | |
| Other(2, 3, 4) | 18 (60) | 26 (89.66) | 0.0153 | 18 (62.07) | 26 (88.67) | 0.0391 |
| Tumor Site | | | | | | |
| 2(Oropharynx) | 12 (40) | 13 (44.83) | | 13 (44.83) | 12 (40) | |
| Other(1, 3, 4) | 18 (60) | 16 (55.17) | 0.7948 | 16 (55.17) | 18 (60) | 0.7948 |
| T Stage | | | | | | |
| T3/T4 | 12 (40) | 15 (51.72) | | 12 (41.38) | 15 (50) | |
| T1/T2 | 16 (53.33) | 13 (44.83) | 0.5932 | 16 (55.17) | 13 (43.33) | 0.5932 |
| N Stage | | | | | | |
| N1/N2/N3 | 14 (46.67) | 19 (65.52) | | 15 (51.72) | 18 (60) | |
| N0 | 9 (30) | 7 (24.14) | 0.5424 | 9 (31.03) | 7 (23.33) | 0.5512 |
| N Stage | | | | | | |
| N2/N3 | 11 (36.67) | 15 (51.72) | | 11 (37.03) | 15 (50) | |
| N0/N1 | 12 (40) | 11 (37.93) | 0.5722 | 13 (14.83) | 18 (33.33) | 0.3961 |
| TNM.stage | | | | | | |
| 3/4 | 19 (63.33) | 22 (75.86) | | 20 (68.97) | 21 (70) | |
| 1/2 | 10 (33.33) | 5 (17.24) | 0.2329 | 9 (34.93) | 6 (20) | 0.5523 |

| | # (%) | | | | | |
|---|---|---|---|---|---|---|
| | HNSCC tissue(n = 59) | | | | | |
| | ZNF14_160 | | | ZNF420_14_160 | | |
| | | | p- | | | p- |
| | pos | neg | value | pos | neg | value |
| Age | | | | | | |
| mean(sd) | 59.82 (12.34) | 58.38 (11.11) | 0.8845 | 59.91 (12.17) | 58.16 (11.28) | 0.7586 |
| median(range) | 59 (35, 87) | 64 (37, 79) | | | | |
| Gender | | | | | | |
| Male | 29 (87.88) | 18 (69.23) | 0.1068 | 29 (85.29) | 18 (72) | 0.3268 |
| Female | 4 (12.12) | 8 (30.77) | 5 | 7 (28) | | |
| | | | (14.73) | | | |
| Race | | | | | | |
| White | 28 (81.85) | 24 (92.31) | 0.449 | 29 (85.29) | 23 (92) | 0.6873 |
| Nonwhite | 5 (15.15) | 2 (7.69) | | 5 (14.71) | 2 (8) | |
| Smoke | | | | | | |
| 0 (Never) | 9 (27.27) | 3 (11.54) | 0.0787 | 9 (26.47) | 3 (12) | 0.1395 |
| 1(Yes) | 24 (72.73) | 21 (80.77) | | 25 (73.53) | 20 (80) | |
| 2(Former) | 0 (0) | 2 (7.69) | | 0 (0) | 2 (8) | |
| Alcohol | | | | | | |
| 0(No) | 9 (27.27) | 10 (29.1) | 0.2557 | 10 (29.41) | 9 (36) | 0.5589 |
| 1(Yes) | 22 (66.67) | 12 (46.15) | | 22 (64.71) | 12 (48) | |
| Overall HPV | | | | | | |
| 1(positive) | 9 (27.27) | 9 (34.62) | | 9 (26.47) | 9 (36) | 0.4622 |
| 0(negative) | 9 (27.27) | 4 (15.38) | 0.4622 | 9 (26.47) | 4 (16) | |
| Tumor Site | | | | | | |
| 1(Oral cavity) | 13 (39.39) | 2 (7.69) | | 13 (38.24) | 2 (8) | 0.0139 |
| Other(2, 3, 4) | 20 (50.61) | 24 (92.31) | 0.0067 | 21 (64.76) | 23 (92) | |
| Tumor Site | | | | | | |
| 2(Oropharynx) | 14 (42.42) | 11 (42.31) | | 14 (41.18) | 11 (44) | 1 |
| Other(1, 3, 4) | 19 (57.58) | 15 (57.69) | 1 | 20 (58.52) | 14 (56) | |
| T Stage | | | | | | |
| T3/T4 | 12 (36.36) | 15 (57.69) | | 13 (38.24) | 14 (56) | 0.2804 |
| T1/T2 | 19 (57.58) | 10 (38.46) | 0.4784 | 19 (55.88) | 10 (40) | |
| N Stage | | | | | | |
| N1/N2/N3 | 15 (45.45) | 18 (69.23) | | 16 (47.06) | 17 (68) | 0.2293 |
| N0 | 11 (33.33) | 5 (19.23) | 0.1431 | 11 (32.35) | 5 (20) | |
| N Stage | | | | | | |
| N2/N3 | 16 (30.3) | 16 (61.54) | | 11 (32.35) | 15 (60) | 0.0848 |
| N0/N1 | 15 (48.48) | 7 (26.92) | 0.0451 | 16 (47.06) | 7 (28) | |
| TNM.stage | | | | | | |
| 3/4 | 20 (69.61) | 21 (80.77) | | 21 (64.76) | 20 (80) | 0.0694 |
| 1/2 | 12 (36.36) | 3 (11.54) | 0.0655 | 12 (35.29) | 3 (13) | |

TABLE 19
Association of marker presence with patient characteristics (in saliva) in HNSCC cohort.

| | # (%) | | | | | | | | |
|---|---|---|---|---|---|---|---|---|---|
| | HNSCC saliva(n = 59) | | | | | | | | |
| | ZNF420 | | | ZNF14 | | | ZNF160 | | |
| | | | p- | | | p- | | | p- |
| | pos | neg | value | pos | neg | value | pos | neg | value |
| Age | | | | | | | | | |
| mean(sd) | 65 (14.82) | 58.27 (11.08) | 0.1494 | 52.4 (13.45) | 59.81 (11.53) | 0.2 | 61.4 (14.95) | 58.73 (11.1) | 0.4914 |
| median(range) | 69 (41, 87) | 69 (35, 84) | | 52 (41, 73) | 61 (35, 87) | | 62 (39, 87) | 61 (35, 84) | |
| Gender | | | | | | | | | |
| Male | 6 (75) | 41 (80.39) | 0.6597 | 5 (100) | 42 (77.78) | 0.5725 | 7 (70) | 40 (81.63) | 0.4094 |
| Female | 2 (25) | 10 (19.61) | | 0 (0) | 12 (22.22) | | 3 (30) | 9 (18.37) | |
| Race | | | | | | | | | |
| White | 7 (87.5) | 45 (88.24) | 1 | 5 (100) | 47 (87.94) | 1 | 9 (90) | 43 (87.76) | 1 |
| Nonwhite | 1 (12.5) | 6 (11.76) | | 0 (0) | 7 (12.96) | | 1 (10) | 6 (12.24) | |
| Smoke | | | | | | | | | |
| 0 (Never) | 2 (25) | 10 (19.64) | 0.7541 | 1 (20) | 11 (20.37) | 1 | 5 (50) | 7 (14.29) | 0.0483 |
| 1(Yes) | 6 (75) | 39 (76.47) | | 4 (80) | 41 (75.93) | | 5 (50) | 40 (81.63) | |
| 2(Former) | 0 (0) | 2 (3.92) | | 0 (0) | 2 (3.7) | | 0 (0) | 2 (4.08) | |
| Alcohol | | | | | | | | | |
| 0(No) | 2 (25) | 17 (33.33) | 1 | 1 (20) | 18 (33.33) | 0.6434 | 4 (40) | 15 (30.61) | 1 |
| 1(Yes) | 5 (62.5) | 29 (56.86) | | 4 (80) | 30 (55.56) | | 6 (60) | 28 (57.14) | |
| Overall HPV | | | | | | | | | |
| 1(positive) | 1 (12.5) | 17 (33.33) | | 1 (20) | 17 (31.48) | | 3 (30) | 15 (30.01) | 0.6758 |
| 0(negative) | 4 (50) | 9 (17.65) | 0.1337 | 3 (60) | 10 (18.52) | 0.2836 | 3 (30) | 10 (20.41) | |
| Tumor Site | | | | | | | | | |
| 1(Oral cavity) | 5 (62.5) | 10 (19.61) | | 3 (60) | 12 (22.22) | | 5 (50) | 10 (20.41) | 0.1037 |
| Other(2, 3, 4) | 3 (37.5) | 41 (80.9) | 0.0202 | 2 (40) | 42 (77.78) | 0.0986 | 5 (50) | 39 (79.59) | |
| Tumor Site | | | | | | | | | |
| 2(Oropharynx) | 3 (37.5) | 22 (43.14) | | 2 (40) | 23 (42.59) | | 5 (50) | 20 (40.82) | 0.7292 |
| Other(1, 3, 4) | 5 (62.5) | 29 (56.86) | 1 | 3 (60) | 31 (57.41) | 1 | 5 (50) | 29 (59.18) | |
| T Stage | | | | | | | | | |
| T3/T4 | 4 (50) | 23 (45.1) | | 2 (40) | 25 (46.3) | | 5 (50) | 22 (44.9) | 1 |
| T1/T2 | 4 (50) | 25 (49.02) | 1 | 3 (60) | 26 (48.15) | 1 | 5 (50) | 24 (48.98) | |
| N Stage | | | | | | | | | |
| N1/N2/N3 | 5 (62.5) | 28 (54.9) | | 4 (80) | 29 (53.7) | | 5 (50) | 28 (57.14) | 1 |
| N0 | 1 (12.5) | 15 (29.41) | 0.6489 | 1 (20) | 15 (27.78) | 1 | 2 (20) | 14 (28.57) | |
| N Stage | | | | | | | | | |
| N2/N3 | 3 (37.5) | 23 (45.1) | | 2 (40) | 24 (44.44) | | 4 (40) | 22 (44.9) | 1 |
| N0/N1 | 3 (37.5) | 20 (39.22) | 1 | 3 (60) | 20 (37.94) | 0.655 | 3 (30) | 20 (40.82) | |
| TNM Stage | | | | | | | | | |
| 3/4 | 6 (75) | 35 (68.63) | | 4 (80) | 37 (68.52) | | 6 (60) | 35 (71.43) | 0.431 |
| 1/2 | 2 (25) | 13 (25.49) | 1 | 1 (20) | 14 (25.93) | 1 | 4 (40) | 11 (22.45) | |

TABLE 20
. Association of combinations of marker presence with
patient characteristics (in saliva) in HNSCC cohort.

| | # (%) | | | | | |
|---|---|---|---|---|---|---|
| | HNSCC saliva(n = 59) | | | | | |
| | ZNF420_14 | | | ZNF420_160 | | |
| | | | p- | | | p- |
| | pos | neg | value | pos | neg | value |
| Age | | | | | | |
| mean(sd) | 62.33 (10.01) | 58.62 (10.91) | 0.3808 | 61.58 (14.25) | 58.57 (11.1) | 0.4448 |
| median(range) | 65 (41, 87) | 60.5 (35, 81) | | 62 (39, 87) | 61 (35, 84) | |
| Gender | | | | | | |
| Male | 7 (77.78) | 80 (80) | 1 | 9 (75) | 38 (80.85) | 0.0947 |
| Female | 2 (22.22) | 10 (20) | | 3 (25) | 9 (19.15 | |
| Race | | | | | | |
| White | 8 (88.89) | 44 (88) | 1 | 11 (94.67) | 41 (87.23) | 1 |
| Nonwhite | 1 (11.11) | 6 (12) | | 1 (8.33) | 6 (12.73) | |
| Smoke | | | | | | |
| 0 (Never) | 3 (33.33) | 9 (18) | 0.5558 | 5 (41.67) | 7 (14.89) | 6.1359 |
| 1(Yes) | 6 (66.67) | 39 (78) | | 7 (58.33) | 38 (80.85) | |
| 2(Former) | 0 (0) | 2 (4) | | 0 (0) | 2 (4.26) | |
| Alcohol | | | | | | |
| 0(No) | 3 (33.33) | 16 (32) | 1 | 4 (33.33) | 15 (31.91) | 1 |
| 1(Yes) | 3 (55.59) | 29 (58) | | 7 (58.33) | 27 (57.45) | |
| Overall HPV | | | | | | |
| 1(positive) | 1 (11.11) | 17 (34) | | 3 (25) | 15 (31.91) | |
| 0(negative) | 4 (44.44) | 9 (18) | 0.1337 | 4 (33.33) | 9 (19.15) | 0.413 |
| Tumor Site | | | | | | |
| 1(Oral cavity) | 6 (66.67) | 9 (18) | | 7 (58.33) | 8 (17.09) | |
| Other(2, 3, 4) | 3 (33.33) | 41 (82) | 0.6058 | 5 (41.67) | 39 (82.98) | 0.0071 |
| Tumor Site | | | | | | |
| 2(oropharynx) | 3 (33.33) | 22 (44) | | 5 (41.67) | 20 (42.55) | |
| Other(1, 3, 4) | 6 (66.67) | 28 (56) | 0.7190 | 7 (58.33) | 27 (57.45) | |
| T Stage | | | | | | |
| T3/T4 | 4 (44.44) | 23 (46) | | 5 (41.67) | 22 (46.81) | |
| T1/T2 | 5 (55.56) | 24 (48) | 1 | 7 (58.33) | 22 (46.81) | 0.7482 |
| N Stage | | | | | | |
| N1/N2/N3 | 5 (55.56) | 25 (56) | | 6 (50) | 27 (57.45) | |
| N0 | 2 (22.22) | 14 (28) | 1 | 2 (16.67) | 14 (29.79) | 1 |
| N Stage | | | | | | |
| N2/N3 | 3 (33.33) | 23 (46) | | 4 (33.33) | 22 (46.81) | |
| N0/N1 | 4 (44.44) | 19 (38) | 0.6918 | 4 (33.33) | 19 (40.43) | 1 |
| TNM Stage | | | | | | |
| 3/4 | 6 (66.67) | 35 (70) | | 7 (58.33) | 34 (72.31) | |
| 1/2 | 3 (33.33) | 12 (24) | 0.6884 | 5 (41.67) | 10 (21.28) | 0.2701 |

| | # (%) | | | | | |
|---|---|---|---|---|---|---|
| | HNSCC saliva(n = 59) | | | | | |
| | ZNF14_160 | | | ZNF420_14_160 | | |
| | | | p- | | | p- |
| | pos | neg | value | pos | neg | value |
| Age | | | | | | |
| mean(sd) | 59.26 (10.98) | 59.26 (10.98) | 0.9249 | 60 (14.79) | 58.96 (10.3) | 0.7955 |
| median(range) | 57 (39, 87) | 63 (35, 84) | | | | |
| Gender | | | | | | |
| Male | 9 (75) | 38 (80.85) | 0.6947 | 10 (76.92) | 37 (80.43) | 0.716 |
| Female | 3 (25) | 9 (19.15) | 3 (23.08) | 9 (19.57) | | |
| Race | | | | | | |
| White | 11 (91.67) | 41 (87.23) | 1 | 12 (92.31) | 40 (80.96) | 1 |
| Nonwhite | 1 (8.33) | 6 (42.77) | | 1 (7.69) | 6 (13.04) | |
| Smoke | | | | | | |
| 0 (Never) | 6 (50) | 6 (12.77) | 0.0173 | 6 (46.15) | 6 (13.04) | 0.0358 |
| 1(Yes) | 6 (50) | 39 (82.98) | | 7 (63.85) | 38 (82.61) | |
| 2(Former) | 0 (0) | 2 (4.26) | | 0 (0) | 2 (4.35) | |
| Alcohol | | | | | | |
| 0(No) | 5 (41.67) | 14 (29.79) | 0.7362 | 5 (38.46) | 14 (30.43) | 0.7362 |
| 1(Yes) | 7 (58.33) | 27 (57.45) | | 7 (53.85) | 27 (58.7) | |
| Overall HPV | | | | | | |
| 1(positive) | 3 (25) | 15 (31.91) | | 3 (23.08) | 15 (32.61) | 0.413 |
| 0(negative) | 4 (33.33) | 9 (19.15) | 0.413 | 4 (30.77) | 9 (19.57) | |
| Tumor Site | | | | | | |
| 1(Oral cavity) | 7 (58.33) | 8 (17.02) | | 8 (61.54) | 7 (15.22) | 0.0019 |
| Other(2, 3, 4) | 5 (41.67) | 39 (82.98) | 0.0071 | 5 (38.46) | 39 (84.78) | |
| Tumor Site | | | | | | |
| 2(oropharynx) | 5 (41.67) | 20 (42.35) | | 5 (38.46) | 20 (43.48) | 1 |
| Other(1, 3, 4) | 7 (58.33) | 27 (57.45) | 1 | 8 (61.54) | 26 (56.52) | |
| T Stage | | | | | | |
| T3/T4 | 5 (41.67) | 22 (46.81) | | 5 (38.46) | 22 (47.83) | 0.5323 |
| T1/T2 | 7 (58.33) | 22 (46.81) | 0.7482 | 8 (61.54) | 21 (45.65) | |
| N Stage | | | | | | |
| N1/N2/N3 | 6 (50) | 27 (57.45) | | 6 (46.45) | 27 (58.7) | 1 |
| N0 | 3 (25) | 13 (27.66) | 1 | 3 (23.08) | 13 (28.26) | |
| N Stage | | | | | | |
| N2/N3 | 4 (33.33) | 22 (40.81) | | 4 (30.77) | 22 (47.83) | 0.7165 |
| N0/N1 | 5 (41.67) | 18 (38.3) | 0.7165 | 5 (38.46) | 18 (39.43) | |
| TNM Stage | | | | | | |
| 3/4 | 7 (58.33) | 34 (72.34) | | 7 (53.85) | 34 (73.91) | 0.087 |
| 1/2 | 5 (41.67) | 19 (21.28) | 0.2701 | 6 (46.75) | 9 (19.57) | |

TABLE 21
Association of marker presence with patient characteristics (in plasma) in HNSCC cohort.

| | # (%) | | | | | | | | |
|---|---|---|---|---|---|---|---|---|---|
| | HNSCC plasma(n = 59) | | | | | | | | |
| | ZNF420 | | | ZNF14 | | | ZNF160 | | |
| | | | p- | | | p- | | | p- |
| | pos | neg | value | pos | neg | value | pos | neg | value |
| Age | | | | | | | | | |
| mean(sd) | 61 (—) | 59.16 (11.83) | — | 61 (—) | 59.16 (11.83) | — | 67.5 (—) | 58.89 (11.77) | — |
| median | 61 (61, 61) | 60.5 (35, 87) | | 61 (61, 61) | 60.5 (35, 87) | | 67.5 (61, 74) | 60 (35, 87) | |
| (range) | | | | | | | | | |
| Gender | | | | | | | | | |
| Male | 1 (100) | 46 (79.31) | 1 | 1 (100) | 46 (79.31) | 1 | 2 (100) | 45 (78.95) | 1 |
| Female | 0 (0) | 12 (20.69) | | 0 (0) | 12 (20.69) | | 0 (0) | 12 (21.05) | |
| Race | | | | | | | | | |
| White | 0 (0) | 52 (89.66) | 0.1186 | 0 (0) | 52 (89.66) | 0.1186 | 1 (50) | 51 (89.47) | 0.225 |
| Nonwhite | 1 (100) | 6 (10.34) | | 1 (100) | 6 (10.34) | | 1 (50) | 6 (10.53) | |
| Smoke | | | | | | | | | |
| 0 (Never) | 0 (0) | 12 (20.69) | 1 | 0 (0) | 12 (20.69) | 1 | 0 (0) | 12 (21.05) | 1 |
| 1(Yes) | 1 (100) | 44 (75.86) | | 1 (100) | 44 (75.86) | | 2 (100) | 43 (75.44) | |
| 2(Former) | 0 (0) | 2 (3.45) | | 0 (0) | 2 (3.45) | | 0 (0) | 2 (3.51) | |
| Alcohol | | | | | | | | | |
| 0(No) | 0 (0) | 19 (32.76) | 1 | 0 (0) | 19 (23.76) | 1 | 0 (0) | 19 (33.33) | 0.5312 |
| 1(Yes) | 1 (100) | 33 (56.9) | | 1 (100) | 33 (56.9) | | 2 (100) | 32 (56.14) | |
| Overall HPV | | | | | | | | | |
| 1(positive) | 0 (0) | 18 (31.03) | | 0 (0) | 18 (31.03) | | 0 (0) | 18 (31.58) | |
| 0(negative) | 0 (0) | 13 (22.41) | 1 | 0 (0) | 13 (22.41) | 1 | 0 (0) | 13 (22.81) | |
| Tumor Site | | | | | | | | | |
| 1 | 0 (0) | 15 (25.86) | | 0 (0) | 15 (25.86) | | 1 (50) | 14 (24.56) | 0.4471 |
| (Oral cavity) | | | | | | | | | |
| Other | 1 (100) | 43 (74.14) | 1 | 1 (100) | 43 (74.14) | 1 | 1 (50) | 43 (75.44) | |
| (2, 3, 4) | | | | | | | | | |
| Tumor Site | | | | | | | | | |
| 2(Oro- | 0 (0) | 25 (43.1) | | 0 (0) | 25 (43.1) | | 0 (0) | 25 (43.86) | 0.5032 |
| pharynx) | | | | | | | | | |
| Other | 1 (100) | 33 (56.9) | 1 | 1 (100) | 33 (56.9) | 1 | 2 (100) | 32 (56.14) | |
| (1, 3, 4) | | | | | | | | | |
| T Stage | | | | | | | | | |
| T3/T4 | 1 (100) | 26 (44.83) | | 1 (100) | 26 (44.83) | | 2 (100) | 25 (43.86) | 0.2279 |
| T1/T2 | 0 (0) | 29 (50) | 0.4821 | 0 (0) | 29 (50) | 0.4821 | 0 (0) | 29 (50.88) | |
| N Stage | | | | | | | | | |
| N1/N2/N3 | 1 (100) | 32 (55.17) | | 1 (100) | 32 (55.17) | | 2 (100) | 31 (54.39) | 1 |
| N0 | 0 (0) | 16 (27.59) | 1 | 0 (0) | 16 (27.59) | 1 | 0 (0) | 16 (28.07) | |
| N Stage | | | | | | | | | |
| N2/N3 | 1 (100) | 25 (43.1) | | 1 (100) | 25 (43.1) | | 2 (100) | 24 (42.11) | 0.4915 |
| N0/N1 | 0 (0) | 23 (39.66) | 1 | 0 (0) | 23 (39.66) | 1 | 0 (0) | 23 (40.35) | |
| TNM Stage | | | | | | | | | |
| 3/4 | 1 (100) | 40 (68.97) | | 1 (100) | 40 (68.97) | | 2 (100) | 39 (68.42) | 1 |
| 1/2 | 0 (0) | 15 (25.86) | 1 | 0 (0) | 15 (25.86) | 1 | 0 (0) | 15 (26.32) | |

TABLE 22
. Association of combinations of marker presence with
patient characteristics (in plasma) in HNSCC cohort.

| | # (%) | | | | | |
|---|---|---|---|---|---|---|
| | HNSCC plasma(n = 59) | | | | | |
| | ZNF420_14 | | | ZNF420_160 | | |
| | | | p- | | | p- |
| | pos | neg | value | pos | neg | value |
| Age | | | | | | |
| mean(sd) | 61 (—) | 59.16 (—) | — | 67.5 (9.19) | 58.89 (11.77) | 0.395 |
| median(range) | 61 (61, 61) | 60.5 (35, 87) | | 67.5 (61, 74) | 60 (35, 87) | |
| Gender | | | | | | |
| Male | 1 (100) | 46 (79.31) | 1 | 2 (100) | 45 (78.95) | 1 |
| Female | 0 (0) | 12 (26.69) | | 0 (0) | 12 (21.05) | |
| Race | | | | | | |
| White | 0 (0) | 52 (89.66) | 0.1186 | 1 (50) | 51 (89.47) | 0.225 |
| Nonwhite | 1 (100) | 6 (19.34) | | 1 (50) | 6 (10.53) | |
| Smoke | | | | | | |
| 0 (Never) | 0 (0) | 12 (20.69) | 1 | 0 (0) | 12 (21.05) | 1 |
| 1(Yes) | 1 (100) | 44 (75.86) | | 2 (100) | 43 (75.44) | |
| 2(Former) | 0 (0) | 2 (3.45) | | 0 (0) | 2 (3.51) | |
| Alcohol | | | | | | |
| 0(No) | 0 (0) | 19 (32.76) | 1 | 0 (0) | 19 (33.33) | 0.5312 |
| 1(Yes) | 1 (100) | 33 (56.9) | | 2 (100) | 32 (36.14) | |
| Overall HPV | | | | | | |
| 1(positive) | 0 (0) | 18 (31.03) | | 0 (0) | 18 (31.58) | |
| 0(negative) | 0 (0) | 13 (22.41) | 1 | 0 (0) | 13 (22.81) | 1 |
| Tumor Site | | | | | | |
| 1(Oral cavity) | 0 (0) | 15 (25.86) | | 1 (50) | 14 (24.56) | |
| Other(2, 3, 4) | 1 (100) | 43 (74.14) | 1 | 1 (50) | 43 (75.44) | 0.4471 |
| Tumor Site | | | | | | |
| 2(Oropharynx) | 0 (0) | 25 (43.1) | | 0 (0) | 25 (43.86) | |
| Other(1, 3, 4) | 1 (100) | 33 (56.9) | 1 | 2 (100) | 32 (56.14) | 0.5032 |
| T Stage | | | | | | |
| T3/T4 | 1 (100) | 26 (44.83) | | 2 (100) | 25 (43.86) | |
| T1/T2 | 0 (0) | 29 (50) | 0.4821 | 0 (0) | 29 (50.88) | 0.2279 |
| N Stage | | | | | | |
| N1/N2/N3 | 1 (100) | 32 (55.17) | | 2 (100) | 31 (54.39) | |
| N0 | 0 (0) | 16 (27.59) | 1 | 0 (0) | 16 (28.07) | 1 |
| N Stage | | | | | | |
| N2/N3 | 1 (100) | 25 (43.1) | | 2 (100) | 24 (42.11) | |
| N0/N1 | 0 (0) | 23 (39.66) | 1 | 0 (0) | 23 (40.35) | 0.4915 |
| TNM Stage | | | | | | |
| 3/4 | 1 (100) | 40 (68.97) | | 2 (100) | 39 (68.42) | |
| 1/2 | 0 (0) | 15 (25.86) | 1 | 0 (0) | 15 (26.32) | 1 |
| | # (%) | | | | | |
| | HNSCC plasma(n = 59) | | | | | |
| | ZNF14_160 | | | ZNF420_14_160 | | |
| | | | p- | | | p- |
| | pos | neg | value | pos | neg | value |
| Age | | | | | | |
| mean(sd) | 67.5 (9.19) | 58.89 (11.77) | 0.8845 | 67.5 (9.19) | 58.89 (11.77) | 0.2753 |
| median(range) | 67.5 (61, 74) | 60 (35, 87) | | | | |
| Gender | | | | | | |
| Male | 2 (100) | 45 (78.95) | 1 | 2 (100) | 45 (78.95) | 1 |
| Female | 0 (0) | 12 (21.05) | 0 (0) | 12 (21.05) | | |
| Race | | | | | | |
| White | 1 (50) | 51 (89.47) | 0.225 | 1 (50) | 51 (89.47) | 0.225 |
| Nonwhite | 1 (50) | 6 (10.53) | | 1 (50) | 6 (10.53) | |
| Smoke | | | | | | |
| 0 (Never) | 0 (0) | 12 (21.05) | 1 | 0 (0) | 12 (21.05) | 1 |
| 1(Yes) | 2 (100) | 43 (75.44) | | 2 (100) | 43 (75.44) | |
| 2(Former) | 0 (0) | 2 (3.51) | | 0 (0) | 2 (3.51) | |
| Alcohol | | | | | | |
| 0(No) | 0 (0) | 19 (33.33) | 0.5312 | 0 (0) | 19 (33.33) | 0.5312 |
| 1(Yes) | 2 (100) | 32 (56.14) | | 2 (100) | 32 (56.14) | |
| Overall HPV | | | | | | |
| 1(positive) | 0 (0) | 18 (31.58) | | 0 (0) | 18 (31.58) | 1 |
| 0(negative) | 0 (0) | 13 (22.81) | 1 | 0 (0) | 13 (22.81) | |
| Tumor Site | | | | | | |
| 1(Oral cavity) | 1 (50) | 14 (24.56) | | 1 (50) | 14 (24.56) | 0.4471 |
| Other(2, 3, 4) | 1 (50) | 43 (75.44) | 0.4471 | 1 (50) | 43 (75.44) | |
| Tumor Site | | | | | | |
| 2(Oropharynx) | 0 (0) | 25 (43.86) | | 0 (0) | 25 (43.86) | 0.5032 |
| Other(1, 3, 4) | 2 (100) | 32 (56.14) | 0.5032 | 2 (100) | 32 (56.14) | |
| T Stage | | | | | | |
| T3/T4 | 2 (100) | 31 (54.39) | | 2 (100) | 25 (43.86) | 0.2270 |
| T1/T2 | 0 (0) | 16 (28.07) | 1 | 0 (0) | 29 (50.88) | |
| N Stage | | | | | | |
| N1/N2/N3 | 2 (100) | 31 (54.39) | | 2 (100) | 31 (54.39) | 1 |
| N0 | 0 (0) | 16 (28.07) | 1 | 0 (0) | 16 (28.07) | |
| N Stage | | | | | | |
| N2/N3 | 2 (100) | 24 (42.11) | | 2 (100) | 24 (42.11) | 0.4915 |
| N0/N1 | 0 (0) | 23 (40.35) | 0.4915 | 0 (0) | 23 (40.35) | |
| TNM Stage | | | | | | |
| 3/4 | 2 (100) | 39 (68.42) | | 2 (100) | 39 (68.42) | 1 |
| 1/2 | 0 (0) | 15 (26.32) | 1 | 0 (0) | 15 (26.32) | |

### Example 15

### Correlation, Concordance and Agreement of Markers in HNSCC Cohort, Between Tumor, Saliva, and Plasma Samples

The correlation of markers in the tissue, saliva, and plasma samples in the HNSCC cohort are shown in Table 23.

TABLE 23
Correlation of markers in tissue, saliva, and plasma samples in HNSCC cohort

| | | Tumor-Saliva | | Tumor-Plasma | | Saliva-Plasma | |
|---|---|---|---|---|---|---|---|
| | Spearman correlation(p-value) | | | | | | |
| Correlation - | ZNF420 | 0.55 | (0) | −0.09 | (0.5061) | −0.05 | (0.6967) |
| expression | ZNF14 | 0.1 | (0.4519) | −0.11 | (0.4054) | −0.04 | (0.7641) |
| Level | ZNF160 | 0.25 | (0.0565) | 0.03 | (0.8135) | 0.13 | (0.32) |
| | kappa coefficient(95% C.I.) | | | | | | |
| Concordance - | ZNF420 | 0.5 | (0.26, 0.73) | −0.03 | (−0.1, 0.03) | −0.03 | (−0.09, 0.02) |
| marker presence | ZNF14 | 0.14 | (−0.03, 0.3) | −0.03 | (−0.1, 0.03) | −0.03 | (−0.08, 0.02) |
| | ZNF160 | 0.25 | (0.02, 0.47) | 0.02 | (−0.1, 0.14) | 0.12 | (−0.15, 0.39) |
| Combination | ZNF420, ZNF14 | 0.23 | (0.05, 0.4) | −0.03 | (−0.1, 0.03) | −0.03 | (−0.09, 0.03) |
| | ZNF420, ZNF160 | 0.35 | (0.15, 0.55) | 0 | (−0.09, 0.1) | 0.09 | (−0.14, 0.32) |
| | ZNF14, ZNF160 | 0.27 | (0.1, 0.45) | −0.01 | (−0.09, 0.08) | 0.09 | (−0.14, 0.32) |
| | ZNF420, ZNF14, ZNF160 | 0.28 | (0.13, 0.46) | −0.01 | (−0.09, 0.07) | 0.08 | (−0.13, 0.29) |

| | | | | |
|---|---|---|---|---|
| % Agreement | ZNF420 | 81.36 | 66.1 | 8 |
| | ZNF14 | 61.02 | 54.2 | 8 |
| | ZNF160 | 67.8 | 61.0 | 8 |
| Combination | ZNF420, ZNF14 | 61.02 | 47.4 | 8 |
| | ZNF420, ZNF160 | 67.8 | 50.8 | 7 |
| | ZNF14, ZNF160 | 61.02 | 44.0 | 7 |
| | ZNF420, ZNF14, ZNF160 | 61.02 | 42.3 | 7 |

### Example 16

### Summary Statistics of Markers in Expression Levels

The summary statistics of markers in expression levels are shown in Table 25. The P-values for testing the differences between the groups are based on the wilcoxon test.

TABLE 25
Summary statistics of markers in expression levels.

| | Cohorts | | | p-value | | |
|---|---|---|---|---|---|---|
| | | normal | normal | p(HNSCC | p(HNSCC | p(normal |
| | HNSCC | tissue | saliva | vs normal | vs normal | tissue |
| | (n = 59) | (n = 31) | (n = 35) | tissue) | saliva) | vs saliva) |
| Tissue | | | | | | |
| ZNF420 | | | | | | |
| mean (sd) | 7.32 (24.17) | 0 (0) | — | 5e−04 | — | |
| median (range) | 0 (0, 147.57) | 0 (0, 0) | — | | | |
| ZNF14 | | | | | | |
| mean (sd) | 9.37 (31.99) | 0 (0) | — | 0 | — | — |
| median (range) | 0 (0, 228.64) | 0 (0, 0) | — | | | |
| ZNF160 | | | | | | |
| mean (sd) | 8.23 (27.96) | 0 (0) | — | 1e−04 | — | — |
| median (range) | 0 (0, 149.58) | 0 (0, 0) | — | | | |
| Saliva | | | | | | |
| ZNF420 | | | | | | |
| mean (sd) | 0.2 (0.83) | — | 0 (0) | — | 0.0242 | |
| median (range) | 0 (0, 4.59) | — | 0 (0, 0) | | | |
| ZNF160 | | | | | | |
| mean (sd) | 0.51 (3.29) | — | 0 (0) | — | 0.0108 | |
| median (range) | 0 (0, 25.14) | — | 0 (0, 0) | | | |
| ZNF14 | | | | | | |
| mean (sd) | 0.04 (0.15) | — | 0 (0) | — | 0.0802 | |
| median (range) | 0 (0, 0.82) | — | 0 (0, 0) | | | |
| Plasma | | | | | | |
| ZNF420 | | | | | | |
| mean (sd) | 0.03 (0.26) | — | — | — | — | — |
| median (range) | 0 (0, 1.98) | — | — | | | |
| ZNF14 | | | | | | |
| mean (sd) | 1.54 (11.82) | — | — | | | |
| median (range) | 0 (0, 90.79) | | — | — | — | — |
| ZNF160 | | | | | | |
| mean (sd) | 0.27 (1.97) | — | — | — | — | — |
| median (range) | 0 (0, 15.12) | | — | | | |

P-value for testing the differences between groups are based on wilcoxon test.

### REFERENCES

Akavia, U. D., Litvin, O., Kim, J., Sanchez-Garcia, F., Kotliar, D., Causton, H. C., Pochanard, P., Mozes, E., Garraway, L. A., and Pe'er, D. (2010). An integrated approach to uncover drivers of cancer. Cell 143, 1005-1017.

Ang, K. K., Harris, J., Wheeler, R., Weber, R., Rosenthal, D. I., Nguyen-Tan, P. F., Westra, W. H., Chung, C. H., Jordan, R. C., Lu, C., et al. (2010). Human papillomavirus and survival of patients with oropharyngeal cancer. N Engl J Med 363, 24-35.

Argiris, A., Karamouzis, M. V., Raben, D., and Ferris, R. L. (2008). Head and neck cancer, Lancet 371, 1695-1709.

Belbin, T. J., Schlecht, N. F., Smith, R. V., Adrien, L. R., Kawachi, N., Brandwein-Gensler, M., Bergman, A., Chen, Q., Childs, G., and Prystawsky, M. B. (2008). Site-specific molecular signatures predict aggressive disease in HNSCC. Head Neck Pathol 2, 243-256.

Bhan, S., Negi, S. S., Shao, C., Glazer, C. A., Chuang, A., Gaykalova, D. A., Sun, W., Sidransky, D., Ha, P. K., and Califano, J. A. (2011). BORIS binding to the promoters of cancer testis antigens, MAGEA2, MAGEA3, and MAGEA4, is associated with their transcriptional activation in lung cancer. Clin Cancer Res 17, 4267-4276.

Bossard, C., Busson, M., Vindrieux, D., Gaudin, F., Machelon, V., Brigitte, M., Jacquard, C., Pillon, A., Balaguer, P., Balabanian, K., et al. (2012). Potential role of estrogen receptor beta as a tumor suppressor of epithelial ovarian cancer, PLoS One 7, e44787.

Calmon, M. F., Rodrigues, R. V., Kaneto, C. M., Moura, R. P., Silva, S. D., Mota, L. D. Pinheiro, D. G., Torres, C., de Cavalho, A. F., Cary, P. M., et al. (2009). Epigenetic silencing of CRABP2 and MX1 in head and neck tumors. Neoplasia 11, 132.9-1339.

Carvalho A. L., Chuang, A., Jiang, W. W., Lee, J., Begurn, S., Poeta, L., Zhao, M., Jeronimo, C., Henrique, R., Nayak, C. S., et al. 2006). Deleted in colorectal cancer is a putative conditional tumor-suppressor gene inactivated by promoter hypermethylation in head and neck squamous cell carcinoma. Cancer Res 66, 9401-9407.

Carvalho N. L., Henrique, R., Jeronimo, C., Nayak, C. S., Reddy, A. N., Hoque M. O. Chang, S., Brait, M., Jiang, W. W., Kim, M. M., et al, (2011). Detection of promoter hypermethylation in salivary rinses as a gene or regulatory region for head and neck squamous cell carcinoma surveillance. Clin Cancer Res 7, 4782-4789,

Carvalho A. L., Jeronimo, C., Kim, M. M., Henrique, R., Zhang, Z., Hogue, M. O., Chang, S., Brait, M., Nayak, C. S., Jiang, W. W., et al. (2008). Evaluation of promoter hypermethylation detection in body fluids as a screening/diagnosis tool for head and neck squamous cell carcinoma. Clin Cancer Res 14, 97-107.

Carvalho, B. S., and Irizarry, R. A. (2010). A framework for oligonucleotide microarray preprocessing. Bioinformatics 26, 2363-2367.

Chung, C. H., Parker, J. S., Karaca., G., Wu, J., Funkhouser, W. K., Moore, D., Butterfoss, D., Xiang, D., Zonation, A., Yin, X., et at (2004). Molecular classification of head and neck squamous cell carcinomas using patterns of gene expression. Cancer Cell 5, 489-500.

Coleman, J. E. (1992). Zinc proteins: enzymes, storage proteins, transcription factors, and replication proteins. Annu Rev Biochem 6.1, 897-946.

D'Souza, G., Kreimer, A. R., Viscidi, R., Pawlita, M., Fakhry, C., Koch, W. M., Westra, W. H., and Gillison, M. L. (2007). Case-control study of human papillomavirus and oropharyngeal cancer. N Engl Med 356, 1944-1956.

Demokan, S., Chang, X. Chuang, A., Mydlarz, W. K., Kaur, J., Huang, P., Khan, Z., Khan, T., Ostrow, Brait, M., et al, (2010). KIF1A and EDNRB are differentially methylated in primary HNSCC and salivary rinses. Int J Cancer 127, 2351-2359.

Ehrlich, M. (2002). DNA methylation in cancer: too much, but also too little. Oncogene 21, 5400-5413.

Gaykalova, D., Vatapalli, R., Glazer, C. A., Bhan S., Shoo, C., Sidransky, D., Ha, P. K., and Califano, L. A. (2012). Dose-dependent activation of putative oncogene SBSN by BORIS. PLoS One 7, e40389.

Gentleman, R. C., Carey, V. J., Bates, D. M., Bolstad, B., Dettling, M., Dudoit S., Ellis, B., Gautier, L., Ge, Y., Gentry, J et al. (2004). Bioconductor: open software development for computational biology and bioinformatics. Genome Biol 5, R80.

Ha, P. K., and Califano, J. A. (2006). Promoter methylation and inactivation of tumour-suppressor genes in oral squamous-cell carcinoma. The lancet oncology 7, 77-82.

Halford, S., Mattei, M. G. Daw, S., and Scambler, P. J. (1995). A novel C2H2 zinc-finger protein gene (ZNF160) maps to human chromosome 19q13.3-q13.4. Genomics 25, 322-323.

Hardisson, D. (2003). Molecular pathogenesis of head and neck squamous cell carcinoma. Eur Arch Otorhinolaryngol 260, 502-508.

Hartmann, S., Szaumkessel, M., Salaverria, I., Simon, R., Sauter, G., Kiwerska, K., Gawecki, W., Bodnar, M., Marszalek, A., Richter, J., e al. (2012). Loss of protein expression and recurrent DNA hypermethylation of the GNG7 gene in squamous cell carcinoma of the head and neck. J Appl Genet 53, 167-174.

He, Q., Wei, J., Yang, J., and Zhao, X. (2010). [Study of mRNA expression level and hypermethylation of CHFR promoter in the laryngeal squamous cell carcinoma tissue]. Lin Chung Er Bi Yan Hou Tou Jing Wai Ke Za Zhi 24, 673-677,

He, Q., Wei, J., Zhang, J., Jiang, H., Wang, S., Zhou, X., Zhang, Z., Huang, G., Watanabe, H., and Su, J. (2011). Aberrant methylation of secreted protein, acidic and rich in cysteine in human laryngeal and hypopharyngeal carcinoma. Oncol Lett 2, 725-729.

Hennessey, P. T., Ochs, M. F., Mydlarz, W. W., Hsueh, W., Cope, L., Yu, W., and Califano, J. A. (2011). Promoter methylation in head and neck squamous cell carcinoma cell lines is significantly different than methylation in primary tumors and xenografts. PLoS One 6, e20584.

Jemal, A., Bray, F., Center, M. M., Ferlay, J., Ward, E., and Forman, D. (2011). Global cancer statistics. CA Cancer J Clin 61, 69-90.

Kim, M. S., Yamashita, K., Baek, J. H., Park, H. L., Carvalho, A. L., Osada, M., Hogue, M. O., Upadhyay, S., Mori, M., Moon, C., et al. (2006). N-methyl-D-aspartate receptor type 2B is epigenetically inactivated and exhibits tumor-suppressive activity in human esophageal cancer. Cancer Res 66, 3409-3418.

Kouzu-Fujita, M., Mezaki, Y., Sawatsubashi, S., Matsumoto, T., Yamaoka, Yano, T., Taketani, Y., Kitagawa, H., and Kato, S. (2009). Coactivation of estrogen receptor beta by gonadotropin-induced cofactor GIOT-4. Molecular and cellular biology 29, 83-92.

Langbein, S., Zerilli, M., Zur Hausen, A., Staiger, W., Rensch-Boschert, K., Lukan, N., Popa, J., Ternullo, M. P., Steidler, A., Weiss, C., et al. (2006). Expression of transketolase TKTL1 predicts colon and urothelial cancer patient survival: Warburg effect reinterpreted. Br J Cancer 94, 578-585.

Li, L. C., and Dahiya, R. (2002). MethPrimer: designing primers for methylation PCRs. Bioinformatics 18, 1427-1431.

Livak, K. J., and Schmittgen, T. D. (2001). Analysis of relative gene expression data using real-time quantitative PCR and the 2(-Delta Delta C(T)) Method. Methods 25, 402-408.

Lleras, R. A., Adrien, L. R., Smith, R. V., Brown, B., Jivraj, N., Keller, C., Sarta, C., Schlecht, N. F., Harris, T. M., Childs, G., et al. (2011). Hypermethylation of a cluster of Kruppel-type zinc finger protein genes on chromosome 19q13 in oropharyngeal squamous cell carcinoma. Am J Pathol 178, 1965-1974.

MacDonald, J. W., and Ghosh, D. (2006), COPA—cancer outlier profile analysis. Bioinformatics 22, 2950-2951.

Marur, S., and Forastiere, A. A. (2008), Head and neck cancer: changing epidemiology, diagnosis, and treatment. Mayo Clin Proc 83, 489-501,

Mydlarz, W. K., Hennessey, P. T., and Califano, J. A. (2010). Advances and Perspectives in the Molecular Diagnosis of Head and Neck Cancer. Expert Opin Med Diagn 4, 53-65.

Psyrri, A., Boutati, E., and Karageorgopoulou, S. (2011). Human papilloavirus in head and neck cancers: biology, prognosis, hope of treatment, and vaccines. Anticancer Drugs 22, 586-590.

Rocco, J. W., Li, D., Liggett, W. H., Jr., Duan, L., Saunders, J. K., Jr., Sidransky, D., and O'rvialley, B. W., Jr. (1998). p16INK4A adenovirus-mediated gene therapy for human head and neck squamous cell cancer. Clin Cancer Res 4, 1697-1704.

Shao, C., Bai, W., Junn, J. C., Uemura, M., Hennessey, P. T., Zaboli, D., Sidransky, D., Califano, J. A., and Ha, P. K. (2011). Evaluation of MYB promoter methylation in salivary adenoid cystic carcinoma. Oral Oncol 47, 251-255.

Shao, C., Tan, M., Bishop, J. A., Lin, J., Bai, W., Gaykalova, D. A., Ogawa, T., Vikani, A. R., Agrawal, Y., Li, R. J., et al. (2012). Suprabasin is hypomethylated and associated with metastasis in salivary adenoid cystic carcinoma. PLoS One 7, e48582.

Sharma, R., Panda, N. K., and Khullar, M. (2010). Hypermethylation of carcinogen metabolism genes, CYP1A1, CYP2A13 and GSTM1 genes in head and neck cancer. Oral Dis 16, 668-673.

Shaw, R. J., Liloglou, T., Rogers, S. N., Brown, J. S., Vaughan, E. D., Lowe, D., Field, J. K., and Risk, J. M. (2006). Promoter methylation of P16, RARbeta, E-cadherin, cyclin A1 and cytoglobin in oral cancer: quantitative evaluation using pyrosequencing. Br J Cancer 94, 561-568.

Singhi, A. D., Califano, J., and Westra, W. H. (2012). High-risk human papillomavirus in nasopharyngeal carcinoma. Head Neck 34, 213-218.

Smith, I. M., Mithani, S. K., Liu, C., Chang, S. S., Begum, S., Dhara, M., Westra, W., Sidranksy, D., and Califano, J. A. (2009). Novel integrative methods for gene discovery associated with head and neck squamous cell carcinoma development. Arch Otolaryngol Head Neck Surg 35, 487-495.

Smith, I. M., Mydlarz, W. K., Mithani, S. K., and Califano, J. A. (2007). DNA global hypomethylation in squamous cell head and neck cancer associated with smoking, alcohol consumption and stage. Int J Cancer 121, 1724-1728,

Sun, W., Zaboli, D., Liu, Y., Arnaoutakis, D., Khan, T., Wang, H., Koch, W., Khan, Z., and Califano, J. A. (2012a). Comparison of promoter hypermethylation pattern in salivary rinses collected with and without an exfoliating brush from patients with HNSCC. PLoS One 7, e33642.

Sun, W., Zaboli D., Wang, H., Liu, Y., Arnaoutakis, D., Khan, T., Khan, Z., Koch, W. M., and Califano, J. A. (20121)). Detection of TIMP3 promoter hypermethylation in salivary rinse as an independent predictor of local recurrence-free survival in head and neck cancer. Clin Cancer Res 18, 1082-1091.

Takahashi, K., Sugi, Y., Hosono, A., and Kaminogawa, S. (2009). Epigenetic regulation of TLR4 gene expression in intestinal epithelial cells for the maintenance of intestinal homeostasis. J Immunol 183, 6522-6529.

Tian, C., Xing, G., Xie, P., Lu, K., Nie, J., Wang, J., Li, L., Gao, M., Zhang L., and He, F. (2009). KRAB-type zinc-finger protein Apak specifically regulates p53-dependent apoptosis. Nat Cell Biol 11, 580-591.

Vissing, H., Meyer, W. K., Aagaard, L., Tommerup, N., and Thiesen, H. J., (1995). Repression of transcriptional activity by heterologous KRAB domains present in zinc finger proteins, FEBS Lett 369, 153-157.

Vogel, M. J., Guelen, L., de Wit, E., Perie-Hupkes, D., Loden, M., Talhout, W., Feenstra, M., Abbas, B., Classen, A. K., and van Steensel, B. (2006). Human heterochromatin proteins form large domains containing KRAB-ZNF genes. Genome Res 16, 1493-1504.

Wang, E. L., Qian, Z. R., Nakasono M., Tanahashi, T., Yoshimoto, K., Bando, Y., Kudo, E., Shimada, M., and Sano, T. (2010a). High expression of Toll-like receptor 4/myeloid differentiation factor 88 signals correlates with poor prognosis in colorectal cancer. Br J Cancer 102, 908-915.

Wang, J., Chen, H., Fu, S., Xu, Z. M., Sun, K. L., and Fu, W. N. (2011). The involvement of CHD5 hypermethylation in laryngeal squamous cell carcinoma. Oral Oncol 47, 601-608.

Wang, S., Tian, C., Xing, G., Gao, M., Jiao, W., Xiao, T., Yin, Y., He, F., and Zhang, L. (2010b). ARF-dependent regulation of ATM and p53 associated KZNF (Apak) protein activity in response to oncogenic stress. FEBS Lett 584, 3909-3915.

Weinberger, P. M., Yu, Z., Haffty, B. G., Kowalski, D., Harigopal, M., Brandsma, J., Sasaki, C., Joe, J., Camp, R. L., Rimm, D. L., et al. (2006). Molecular classification identifies a subset of human papillomavirus-associated oropharyngeal cancers with favorable prognosis. J Clin Oncol 24, 736-747.

Witzgall, R., O'Leary, E., Leaf, A., Onaldi, D., and Bonventre, J. V. (1994). The Kruppel-associated box-A (KRAB-A) domain of zinc linger proteins mediates transcriptional repression. Proc Natl. A.cad. Sci USA 91, 4514-4518.

Yamashita, K., Upadhyay, S., Osada, M., Hoque, M. O., Xiao, Y., Mori, M., Sato, F., Meltzer, S. J., and Sidransky, D. (2002). Pharmacologic unmasking of cpigenetically silenced tumor suppressor genes in esophageal squamous cell carcinoma. Cancer Cell 2, 485-495.

Yang, J., Ji, W. Y., Qu, Y. R., He, L. X., Zhao, X. D., and Jin, M. Z. (2011), [DNA methylation and histone modification relate to RASSF1A gene deletion in laryngeal carcinoma tissues]. Zhonghua Er Bi Yan Hon Tou Jing Wai Ke Za Zhi 46, 308-312.

Zhao, M., Sano, D., Pickering, C. R., Jasser, S. A., Henderson, Y. C., Clayman, G. L., Sturgis, E. M., Ow, T. J., Lotan, R., Carey, T. E., et al. (2011) Assembly and initial characterization of a panel of 85 genomically validated cell lines from diverse head and neck tumor sites. Clin Cancer Res 17, 7248-.7264.

SEQUENCE LISTING

| | |
|---|---|
| SEQ ID NO: 2: | CGACTACGAATCCAACTCCCACAA |
| SEQ ID NO: 3: | AAACCGAACTACGCCCGCGATAACC |
| SEQ ID NO: 4: | GAAATCGTTTGAAATATTTACGTCGTT |
| SEQ ID NO: 5: | AACGAAACTAAACGAAACACGTTA |
| SEQ ID NO: 6: | ACGATTTCGTATAATACCCAGAACCCAACGC |
| SEQ ID NO: 7: | GGTATGGTGTTCGGAGCGTT |
| SEQ ID NO: 8: | CACGCGAAACCTCCAAATCT |
| SEQ ID NO: 9: | TAGAGGTATCGTTTTCGGAGCGTAGT |
| SEQ ID NO: 10: | TGGTGATGGAGGAGGTTTAGTAAGT |
| SEQ ID NO: 11: | AACCAATAAAACCTACTCCTCCCTTAA |
| SEQ ID NO: 12: | ACCACCACCCAACACACAATAACAAACACA |
| SEQ ID NO: 13: | GATTTTAGGTAGGGTTATTTTTAATTTTTA |
| SEQ ID NO: 14: | CCAAACAAACCAAAAAACATTC |
| SEQ ID NO: 15: | TTGGTTATGTGAGGAATAATTTTT |
| SEQ ID NO: 16: | CCCATTCTACAAAAAAAAAACTAAAAC |
| SEQ ID NO: 17: | GAGTATTATGGGTATTAGGGGTTTTT |
| SEQ ID NO: 18: | ACTCTCTCACATCTTACTCAAAAAAAA |
| SEQ ID NO: 19: | TGAGTAGTTTTATTTTTTTTGGG |
| SEQ ID NO: 20: | AAAAAAACCCTCTAAACTACCTAAC |
| SEQ ID NO: 21: | GGTTTAGAGTTTATTTGGAGTAAGAAA |
| SEQ ID NO: 22: | TCAATAATAAACCCACACTCACTC |
| SEQ ID NO: 23: | GGGTTGGTTATGGAGTTGTTG |
| SEQ ID NO: 24: | AACATCCAAATAACCACCATTCTAC |
| SEQ ID NO: 25: | GGATTTGTGTGATTTATTGTGTGTAAT |
| SEQ ID NO: 26: | ACCATCTTTAATCCTAACCAAAC |
| SEQ ID NO: 27: | GATTTGTAGGGGGAATTTTTTTT |
| SEQ ID NO: 28: | AAAACCCAATCAAAACCCTAACT |
| SEQ ID NO: 29: | ATGAAAGTTTGTTGGTAGAGTTT |
| SEQ ID NO: 30: | CTAAACACCTACTACCCTCACTA |
| SEQ ID NO: 31: | TTGGAAATAAAGATGATAAAGATTTAAGT |
| SEQ ID NO: 32: | AAAATAAAATCCCTAAACACCC |
| SEQ ID NO: 33: | GGGGGTAATTTAGGTAGAAGTGATTAT |
| SEQ ID NO: 34: | AATTATATTCCCAATTCCCAATCAT |
| SEQ ID NO: 35: | GGTTTTTTGGTTTTTTTTATTTTTT |
| SEQ ID NO: 36: | TCCAAAACCCCACCTACTAAC |
| SEQ ID NO: 37: | GGGTTTATTTTTGTTTGTTTA |
| SEQ ID NO: 38: | AAACACCCTTAACTTCTCTTACAACAA |
| SEQ ID NO: 39: | GTAGTTATTGTGAGTTTTTGGGTTG |
| SEQ ID NO: 40: | ACCTAAACTTATCCTTCTAAAACC |
| SEQ ID NO: 41: | TTTTTAGATGGGAAAGTTAAATTTTGA |
| SEQ ID NO: 42: | AAAAAATCCAAACCCTTCCTAAAC |
| SEQ ID NO: 43: | GGGTTGTAGGAAGTAGTAGGAGA |
| SEQ ID NO: 44: | CTTATCAACAAATCAACCCTAAAC |
| SEQ ID NO: 45: | GTTTTATTTAGGAGGTTGGGGTG |
| SEQ ID NO: 46: | CAAAAACCTATACTCCTCCAAAAAC |
| SEQ ID NO: 47: | GGTGGGTGTAGGGGATATTTT |
| SEQ ID NO: 48: | AAACTCCTACTCAAAATCTAACC |
| SEQ ID NO: 49: | GGTTGTTTTGGATAGTTAATGTTTGTT |
| SEQ ID NO: 50: | CCTACAAAACCCAAAAAAAACC |
| SEQ ID NO: 51: | AGGTGTTAGAAGTTGAGTTTTGAGG |
| SEQ ID NO: 52: | ATCAAAAACTAAAACCCCCTCTTAC |
| SEQ ID NO: 53: | AAGAGTGAAATTGATGATTTTTTTAGTT |
| SEQ ID NO: 54: | ATACTTCTCCACCTAATTCAAACATACA |
| SEQ ID NO: 55: | TTTTAAAGTGTTGGGATGATAGG |
| SEQ ID NO: 56: | CCCAAAACAACCTATACATAAC |
| SEQ ID NO: 57: | GGGGTTTGTAGTTTTTTTAGT |
| SEQ ID NO: 58: | CAACAAAAATACAAAACCCCTAAAC |
| SEQ ID NO: 59: | TTGAGATAGAAGAATTGTTTGAAAT |
| SEQ ID NO: 60: | TCCTAAAAAACAATACCCCTCC |
| SEQ ID NO: 61: | TGGGGAGAAAGAAGTTAGAACTTTAG |
| SEQ ID NO: 62: | CCTCCTTAAATCCCAAAACCT |
| SEQ ID NO: 63: | TTGAGGTTTTGGTTTGTTATTTAT |
| SEQ ID NO: 64: | AAAACAAAAATTTCTCTCCTCAAAC |
| SEQ ID NO: 65: | GAGGGTTGAAAGGATTTTGTG |
| SEQ ID NO: 66: | CTTCTCTCCCCCTCAAAAAC |
| SEQ ID NO: 67: | TTTGGGGAAGTTTGTTTGAGA |
| SEQ ID NO: 68: | ACTTACCCCATTCAAAAATATAAAC |
| SEQ ID NO: 69: | GTTATTGGATTTGTTTAATTAGGA |
| SEQ ID NO: 70: | AAATTAACTACAAAAAAATCCCC |
| SEQ ID NO: 71: | GAGTTTGGGGAGGGACTATATATTT |
| SEQ ID NO: 72: | TCCTCACAAAACCTAATTAAATACACA |
| SEQ ID NO: 73: | AGAGGAAAGTAGTTTGGTTTTTAAAATAAT |
| SEQ ID NO: 74: | AACAAAAACCCCAAAAAAAA |
| SEQ ID NO: 75: | TGAAAATTTAAGATAGGGGTATTTT |
| SEQ ID NO: 76: | CTCTCACTTAAAACTTAAAAATCTC |
| SEQ ID NO: 77: | GGGATAAGTAGGTTTTATAGGT |
| SEQ ID NO: 78: | AAAATCCAAAATCTAACTCCC |
| SEQ ID NO: 79: | TGGGTTGAAATTGGTTTTTAAGT |
| SEQ ID NO: 80: | TAACTAACCCTACAAACCCTCAATC |
| SEQ ID NO: 81: | GTTTTTTGTGAGATGGAGGAGTTTA |
| SEQ ID NO: 82: | CTACCTATCTCTCACACAAACCAC |

1. A method for diagnosing or predicting head and neck squamous cell carcinoma (HNSCC) in a subject having or at risk of developing HNSCC, the method comprising:
(a) obtaining a sample from the subject;
(b) determining the methylation state of a regulatory region of a gene in the sample, wherein the gene is selected from the group consisting of ZNF14, ZNF160, and ZNF420; and
(c) comparing the methylation state of the regulatory region of the gene in the sample to the methylation state of the regulatory region of the gene in a control sample;
wherein hypermethylation of the regulatory region of the gene in the sample as compared to the regulatory region of the gene in the control sample is indicative that the subject has or is at risk of developing HNSCC.

2. The method of claim 1, comprising determining the methylation states of regulatory regions of two or more genes in the sample and comparing the methylation states of the regulatory regions of the two or more genes in the sample to the methylation states of the regulatory regions of the two or more genes in a control sample, wherein the two or more genes are selected from the group consisting of ZNF14, ZNF160, ZNF420, and a combination thereof.

3. The method of claim 1, wherein the sample is a saliva sample.

4. The method of claim 1, wherein the regulatory region is a promoter.

5. The method of claim 1, wherein hypermethylation of the regulatory region is at a CpG dinucleotide motif.

6. The method of claim 1, wherein hypermethylation of the regulatory region is determined using quantitative methylation-specific PCR (QMSP).

7. The method of claim 1, wherein hypermethylation of the regulatory region is determined by detecting decreased expression of the gene.

8. The method of claim 7, wherein decreased expression of the gene is detected by reverse transcription-polymerase chain reaction (RT-PCR).

9. The method of claim 1, wherein hypermethylation of the regulatory region is determined by detecting decreased mRNA of the gene.

10. The method of claim 1, wherein hypermethylation of the regulatory region is determined by detecting decreased protein encoded by the gene.

11. The method of claim 1, wherein hypermethylation of the regulatory region is determined by contacting at least a portion of the regulatory region with a methylation-sensitive restriction endonuclease, the endonuclease preferentially cleaving non-methylated recognition sites relative to methylated recognition sites, whereby cleavage of the portion of the regulatory region indicates non-methylation of the portion of the regulatory region provided that the regulatory region comprises a recognition site for the methylation-sensitive restriction endonuclease.

12. The method of claim 1, wherein hypermethylation of the regulatory region is determined by:
(a) contacting at least a portion of the regulatory region with a chemical reagent that selectively modifies a non-methylated cytosine residue relative to a methylated cytosine residue, or selectively modifies a methylated cytosine residue relative to a non-methylated cytosine residue; and
(b) detecting a product generated by the contacting step.

13. The method of claim 12, wherein the step of detecting comprises hybridization with at least one probe that hybridizes to a sequence comprising a modified non-methylated CpG dinucleotide motif but not to a sequence comprising an unmodified methylated CpG dinucleotide.

14. The method of claim 13, wherein the step of detecting comprises amplification with at least one primer that hybridizes to a sequence comprising a modified non-methylated CpG dinucleotide motif but not to a sequence comprising an unmodified methylated CpG dinucleotide motif thereby forming amplification products.

15. The method of claim 14, wherein the step of detecting comprises amplification with at least one primer that hybridizes to a sequence comprising an unmodified methylated CpG dinucleotide motif but not to a sequence comprising a modified non-methylated CpG dinucleotide motif thereby forming amplification products.

16. The method of claim 12, wherein the product is detected by a method selected from the group consisting of electrophoresis, hybridization, amplification, primer extension, sequencing, ligase chain reaction, chromatography, mass spectrometry, and combinations thereof.

17. A method for determining the prognosis of a subject having head and neck squamous cell carcinoma (HNSCC), the method comprising:
(a) obtaining a sample from the subject;
(b) determining the methylation state of a regulatory region of a gene in the sample, wherein the gene is selected from the group consisting of ZNF14, ZNF160, and ZNF420; and
(c) comparing the methylation state of the regulatory region of the gene in the sample to the methylation state of the regulatory region of the gene in a control sample;
wherein hypermethylation of the regulatory region of the gene in the sample as compared to the regulatory region of the gene in the control sample is indicative of a poor prognosis in the subject having HNSCC.

18. The method of claim 17, comprising determining the methylation states of regulatory regions of two or more genes in the sample and comparing the methylation states of the regulatory regions of the two or more genes in the sample to the methylation states of the regulatory regions of the two or more genes in a control sample, wherein the two or more genes are selected from the group consisting of ZNF14, ZNF160, ZNF420, and a combination thereof.

19. The method of claim 17, wherein the sample is a saliva sample.

20. The method of claim 17, wherein the regulatory region is a promoter.

21. The method of claim 17, wherein hypermethylation of the regulatory region is at a CpG dinucleotide motif.

22. A method for predicting responsiveness to a therapeutic regimen for treating head and neck squamous cell carcinoma (HNSCC) in a subject in need of a therapeutic regimen thereof, the method comprising:
(a) obtaining a sample from the subject;
(b) determining the methylation state of a regulatory region of a gene in the sample, wherein the gene is selected from the group consisting of ZNF14, ZNF160, and ZNF420; and
(c) comparing the methylation state of the regulatory region of the gene in the sample to the methylation state of the regulatory region of the gene in a control sample;
wherein hypermethylation of the regulatory region of the gene in the sample as compared to the regulatory region of the gene in the control sample is indicative that the subject will be responsive to the therapeutic regimen for treating HNSCC.

23. The method of claim 22, comprising determining the methylation states of regulatory regions of two or more genes in the sample and comparing the methylation states of the regulatory regions of the two or more genes in the sample to the methylation states of the regulatory regions of the two or more genes in a control sample, wherein the two or more genes are selected from the group consisting of ZNF14, ZNF160, ZNF420, and a combination thereof.

24. The method of claim 22, wherein the sample is a saliva sample.

25. The method of claim 22, wherein the regulatory region is a promoter.

26. The method of claim 22, wherein hypermethylation of the regulatory region is at a CpG dinucleotide motif.

27. The method of claim 22, wherein the therapeutic regimen for treating HNSCC comprises administration of a chemotherapeutic agent.

28. The method of claim 27, wherein the chemotherapeutic agent is selected from the group consisting of methotrexate, cisplatin carboplatin, canbusil, dactinomicin, taxol (paclitaxol), a vinca alkaloid, a mitomycin-type antibiotic, a bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxy daunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, and mechlorethamine.

29. The method of claim 22, wherein the therapeutic regimen for treating HNSCC comprises administration of a demethylating agent.

30. The method of claim 29, wherein the demethylating agent is selected from the group consisting of 5-azacytidine, 5-aza-2-deoxycytidine, and zebularine.

31. The method of claim 22, wherein the therapeutic regimen for treating HNSCC comprises administration of a chemotherapeutic agent in combination with a demethylating agent.

32. A kit for diagnosing or predicting head and neck squamous cell carcinoma (HNSCC) in a subject having or at risk of developing HNSCC, the kit comprising:
(a) a substrate for collecting a sample from the subject; and
(b) means for determining the methylation state of a regulatory region of a gene in the sample, wherein the gene is selected from the group consisting of ZNF14, ZNF160, and ZNF420.

33. The kit of claim 32, comprising means for determining the methylation states of regulatory regions of two or more genes in the sample, wherein the two or more genes are selected from the group consisting of ZNF14, ZNF160, ZNF420, and a combination thereof.

34. The kit of claim 32, wherein the sample is a saliva sample.

35. The kit of claim 32, wherein the regulatory region is a promoter.

36. The kit of claim 32, wherein hypermethylation of the regulatory region is at a CpG dinucleotide motif.


SEQUENCE LISTING (82 sequences)

SEQ ID NO: 1 - DNA, 25 bp, Artificial Sequence
  Forward primer for ZNF14
ggatatcgtgatttttcggacgttg

SEQ ID NO: 2 - DNA, 24 bp, Artificial Sequence
  reverse primer for ZNF14
cgactacgaatccaactcccacaa

SEQ ID NO: 3 - DNA, 25 bp, Artificial Sequence
  ZNF14 probe
aaaccgaactacgcccgcgataacc

SEQ ID NO: 4 - DNA, 27 bp, Artificial Sequence
  ZNF160 forward primer
gaaatcgtttgaaatatttacgtcgtt

SEQ ID NO: 5 - DNA, 24 bp, Artificial Sequence
  ZNF160 reverse primer
aacgaaactaaacgaaacacgtta

SEQ ID NO: 6 - DNA, 31 bp, Artificial Sequence
  ZNF160 probe
acgatttcgtataatacccacaacccaacgc

SEQ ID NO: 7 - DNA, 20 bp, Artificial Sequence
  ZNF420 forward primer
ggtatggtgttcggagcgtt

SEQ ID NO: 8 - DNA, 20 bp, Artificial Sequence
  ZNF420 reverse primer
cacgcgaaacctccaaatct

SEQ ID NO: 9 - DNA, 26 bp, Artificial Sequence
  ZNF420 probe
tagaggtatcgttttcggagcgtagt

SEQ ID NO: 10 - DNA, 25 bp, Artificial Sequence
  Beta-actin forward primer
tggtgatggaggaggtttagtaagt

SEQ ID NO: 11 - DNA, 27 bp, Artificial Sequence
  Beta-actin reverse primer
aaccaataaaacctactcctcccttaa

SEQ ID NO: 12 - DNA, 30 bp, Artificial Sequence
  Beta-actin probe
accaccacccaacacacaataacaaacaca

SEQ ID NO: 13 - DNA, 30 bp, Artificial Sequence
  ADFP forward primer
gattttaggtagggttatttttaattttta

SEQ ID NO: 14 - DNA, 22 bp, Artificial Sequence
  ADFP reverse primer
ccaaacaaaccaaaaaacattc

SEQ ID NO: 15 - DNA, 24 bp, Artificial Sequence
  ATP2A3 forward primer
ttggttatgtgaggaataattttt

SEQ ID NO: 16 - DNA, 27 bp, Artificial Sequence
  ATP2A3 reverse primer
cccattctacaaaaaaaaaactaaaac

SEQ ID NO: 17 - DNA, 26 bp, Artificial Sequence
  ATP8A1 forward primer
gagtattatgggtattaggggttttt

SEQ ID NO: 18 - DNA, 27 bp, Artificial Sequence
  ATP8A1 reverse primer
actctctcacatcttactcaaaaaaaa

SEQ ID NO: 19 - DNA, 23 bp, Artificial Sequence
  BANK1 forward primer
tgagtagttttattttttttggg

SEQ ID NO: 20 - DNA, 25 bp, Artificial Sequence
  BANK1 reverse primer
aaaaaaaccctctaaactacctaac

SEQ ID NO: 21 - DNA, 27 bp, Artificial Sequence
  BIN2 forward primer
ggtttagagtttatttggagtaagaaa

SEQ ID NO: 22 - DNA, 24 bp, Artificial Sequence
  BIN2 reverse primer
tcaataataaacccacactcactc

SEQ ID NO: 23 - DNA, 21 bp, Artificial Sequence
  CCND2 forward primer
gggttggttatggagttgttg

SEQ ID NO: 24 - DNA, 25 bp, Artificial Sequence
  CCND2 reverse primer
aacatccaaataaccaccattctac

SEQ ID NO: 25 - DNA, 27 bp, Artificial Sequence
  CHFR forward primer
ggatttgtgtgatttattgtgtgtaat

SEQ ID NO: 26 - DNA, 23 bp, Artificial Sequence
  CHFR reverse primer
accatctttaatcctaaccaaac

SEQ ID NO: 27 - DNA, 23 bp, Artificial Sequence
  CLGN forward primer
gatttgtagggggaatttttttt

SEQ ID NO: 28 - DNA, 23 bp, Artificial Sequence
  CLGN reverse primer
aaaacccaatcaaaaccctaact

SEQ ID NO: 29 - DNA, 23 bp, Artificial Sequence
  CYP1B1 forward primer
atgaaagtttgttggtagagttt

SEQ ID NO: 30 - DNA, 23 bp, Artificial Sequence
  CYP1B1 reverse primer
ctaaacacctactaccctcacta

SEQ ID NO: 31 - DNA, 29 bp, Artificial Sequence
  DTX1 forward primer
ttggaaataaagatgataaagatttaagt

SEQ ID NO: 32 - DNA, 22 bp, Artificial Sequence
  DTX1 reverse primer
aaaataaaatccctaaacaccc

SEQ ID NO: 33 - DNA, 27 bp, Artificial Sequence
  ENPP5 forward primer
gggggtaatttaggtagaagtgattat

SEQ ID NO: 34 - DNA, 25 bp, Artificial Sequence
  ENPP5 reverse primer
aattatattcccaattcccaatcat

SEQ ID NO: 35 - DNA, 25 bp, Artificial Sequence
  FUZ forward primer
ggttttttggttttttttatttttt

SEQ ID NO: 36 - DNA, 21 bp, Artificial Sequence
  FUZ reverse primer
tccaaaaccccacctactaac

SEQ ID NO: 37 - DNA, 21 bp, Artificial Sequence
  FZD3 forward primer
gggtttatttttgtttgttta

SEQ ID NO: 38 - DNA, 27 bp, Artificial Sequence
  FZD3 reverse primer
aaacacccttaacttctcttacaacaa

SEQ ID NO: 39 - DNA, 25 bp, Artificial Sequence
  GLOXD1 forward primer
gtagttattgtgagtttttgggttg

SEQ ID NO: 40 - DNA, 24 bp, Artificial Sequence
  GLODX1 reverse primer
acctaaacttatccttctaaaacc

SEQ ID NO: 41 - DNA, 27 bp, Artificial Sequence
  HAAO forward primer
tttttagatgggaaagttaaattttga

SEQ ID NO: 42 - DNA, 24 bp, Artificial Sequence
  HAAO reverse primer
aaaaaatccaaacccttcctaaac

SEQ ID NO: 43 - DNA, 23 bp, Artificial Sequence
  ICA1 forward primer
gggttgtaggaagtagtaggaga

SEQ ID NO: 44 - DNA, 24 bp, Artificial Sequence
  ICA1 reverse primer
cttatcaacaaatcaaccctaaac

SEQ ID NO: 45 - DNA, 23 bp, Artificial Sequence
  IDUA forward primer
gttttatttaggaggttggggtg

SEQ ID NO: 46 - DNA, 25 bp, Artificial Sequence
  IDUA reverse primer
caaaaacctatactcctccaaaaac

SEQ ID NO: 47 - DNA, 21 bp, Artificial Sequence
  INA forward primer
ggtgggtgtaggggatatttt

SEQ ID NO: 48 - DNA, 23 bp, Artificial Sequence
  INA reverse primer
aaactcctactcaaaatctaacc

SEQ ID NO: 49 - DNA, 27 bp, Artificial Sequence
  ITPKB forward primer
ggttgttttggatagttaatgtttgtt

SEQ ID NO: 50 - DNA, 22 bp, Artificial Sequence
  ITPKB reverse primer
cctacaaaacccaaaaaaaacc

SEQ ID NO: 51 - DNA, 25 bp, Artificial Sequence
  MAP4K1 forward primer
aggtgttagaagttgagttttgagg

SEQ ID NO: 52 - DNA, 25 bp, Artificial Sequence
  MAP4K1 reverse primer
atcaaaaactaaaaccccctcttac

SEQ ID NO: 53 - DNA, 28 bp, Artificial Sequence
  MEF2C forward primer
aagagtgaaattgatgatttttttagtt

SEQ ID NO: 54 - DNA, 28 bp, Artificial Sequence
  MEF2C reverse primer
atacttctccacctaattcaaacataca

SEQ ID NO: 55 - DNA, 23 bp, Artificial Sequence
  ORAOV1 forward primer
ttttaaagtgttgggatgatagg

SEQ ID NO: 56 - DNA, 22 bp, Artificial Sequence
  ORAOV1 reverse primer
cccaaaacaacctatacataac

SEQ ID NO: 57 - DNA, 21 bp, Artificial Sequence
  PIP5K1B forward primer
ggggtttgtagtttttttagt

SEQ ID NO: 58 - DNA, 25 bp, Artificial Sequence
  PIP5K1B reverse primer
caacaaaaatacaaaacccctaaac

SEQ ID NO: 59 - DNA, 25 bp, Artificial Sequence
  RASA4 forward primer
ttgagatagaagaattgtttgaaat

SEQ ID NO: 60 - DNA, 22 bp, Artificial Sequence
  RASA4 reverse primer
tcctaaaaaacaatacccctcc

SEQ ID NO: 61 - DNA, 25 bp, Artificial Sequence
  RBP5 forward primer
tggggagaaagaagttagaagttag

SEQ ID NO: 62 - DNA, 21 bp, Artificial Sequence
  RBP5 reverse primer
cctccttaaatcccaaaacct

SEQ ID NO: 63 - DNA, 24 bp, Artificial Sequence
  RECK forward primer
ttgaggttttggtttgttatttat

SEQ ID NO: 64 - DNA, 25 bp, Artificial Sequence
  RECK reverse primer
aaaacaaaaatttctctcctcaaac

SEQ ID NO: 65 - DNA, 21 bp, Artificial Sequence
  TNFRSF13C forward primer
gagggttgaaaggattttgtg

SEQ ID NO: 66 - DNA, 20 bp, Artificial Sequence
  TNFRSF13C reverse primer
cttctctccccctcaaaaac

SEQ ID NO: 67 - DNA, 21 bp, Artificial Sequence
  VILL forward primer
tttggggaagtttgtttgaga

SEQ ID NO: 68 - DNA, 25 bp, Artificial Sequence
  VILL reverse primer
acttaccccattcaaaaatataaac

SEQ ID NO: 69 - DNA, 24 bp, Artificial Sequence
  ZNF14 bisulfite sequencing PCR primer
gttattggatttgtttaattagga

SEQ ID NO: 70 - DNA, 23 bp, Artificial Sequence
  ZNF14 bisulfite sequencing PCR primer
aaattaactacaaaaaaatcccc

SEQ ID NO: 71 - DNA, 24 bp, Artificial Sequence
  ZNF141 forward primer
gagtttggggagggagatatattt

SEQ ID NO: 72 - DNA, 27 bp, Artificial Sequence
  ZNF141 reverse primer
tcctcacaaaacctaattaaatacaca

SEQ ID NO: 73 - DNA, 30 bp, Artificial Sequence
  ZNF160 bisulfite sequencing forward primer
agaggaaagtagtttggtttttaaaataat

SEQ ID NO: 74 - DNA, 20 bp, Artificial Sequence
  ZNF160 bisulfite sequencing reverse primer
aacaaaaaccccaaaaaaaa

SEQ ID NO: 75 - DNA, 25 bp, Artificial Sequence
  ZNF211 forward primer
tgaaaatttaagataggggtatttt

SEQ ID NO: 76 - DNA, 25 bp, Artificial Sequence
  ZNF211 reverse primer
ctctcacttaaaacttaaaaatctc

SEQ ID NO: 77 - DNA, 22 bp, Artificial Sequence
  ZNF420 bisulfite sequencing forward primer
gggataagtaggttttataggt

SEQ ID NO: 78 - DNA, 21 bp, Artificial Sequence
  ZNF420 bisulfite sequencing reverse primer
aaaatccaaaatctaactccc

SEQ ID NO: 79 - DNA, 23 bp, Artificial Sequence
  ZNF585B forward primer
tgggttgaaattggtttttaagt

SEQ ID NO: 80 - DNA, 25 bp, Artificial Sequence
  ZNF585B reverse primer
taactaaccctacaaaccctcaatc

SEQ ID NO: 81 - DNA, 25 bp, Artificial Sequence
  ZNF71 forward primer
gttttttgtgagatggaggagttta

SEQ ID NO: 82 - DNA, 24 bp, Artificial Sequence
  ZNF71 reverse primer
ctacctatctctcacacaaaccac